SEQUENCE LISTING (80 sequences)

SEQ ID NO: 1 - protein, 385 aa, Unknown
  polyketide synthase
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - protein, 404 aa, Unknown
  polyketide synthase
MGSAPPAATVQEMRRAQRADGPAAVLAIGTANPPSIMPQDDYPDYYFRVTNSEHLTDLKAKLSRICNHNKSGIRQRYLHLNEELLAANPGFIDPKRPSLDERVEMASAAVPELAAKAAAKAIAEWGRPATDITHLIFSTYSGARAPSGDRRLASLLGLRPTVSRTILSLHGCYGGGRALQLAKELAENNRGARVLVACSELTLIAFYGPEGGCVDNIIGQTLFGDGAGAVIVGADPVGAPAERPLFEMVFASQTTIPETEDAISMQYSKCGMEYHLSSRVPRVLGSNVERCLVDTFRTLGVSVAWNDLFWAIHPGGRAILDNIEEVLRLEDGKLAASRHVLSEFGNMSGTTVIFVLDELRRRRAAAAKQGGQAPEWGVMMAFGPGITVETMVLHAPSNLELEGN

SEQ ID NO: 3 - protein, 399 aa, Unknown
  polyketide synthase
MGSAPATVQEMRRAQRADGPAAVLGIGTANPPTCLAQDDYPDYYFRVTNSEHLTDLKGKLTRICNKSGIKQRYIHLNEDLLAANPDFTDRTRPSLDARVDIASAAVPELAAAAAAKAIAEWGRPATDITHLVFSTYSGARAPSADRRLASLLGLRPTVSRTILNLHGCYGGGRSLQLAKELAENNRGARVLVACSEITLIAFYGPEGGCADNILGQALFGDGAGAVIVGADPVAPVERPLYEMAFASQTTIPETEDAISMQINKGGMEYHISNQVPRLLGCNVERCLVDAFRALGVSAAWNDLFWAIHPGGRAILDHIEGVLGLDDTKLAASRHVLSEFGNMSGTTVIFVLDELRRRRAAMAKQGGEAPEWGVMMAFGPGITIETMVLHAPSNLDLKGN

SEQ ID NO: 4 - protein, 399 aa, Unknown
  polyketide synthase
MGSAPATAQEMRRAQRADGPAAVLGIGTANPPTCLAQDDYPDYYFRVTNSEHLTDLKGKLTRICNKSGIKQRYIHLNEDLLAANPDFADRTRPSLDARVDIASAAVPELAAAAAAKAIAEWGRPATDITHLVFSTYSGARAPSADRRLASLLGLRPTVSRTILNLHGCYGGGRSLQLAKELAENNRGARVLVACSEITLIAFYGPEGGCADNILGQALFGDGAGAVIVGADPVAPVERPLFEMAFASQTTIPETEDAISMQINKGGMEYHISNQVPRLLGCNVERCLVDAFRALGVSAAWNDLFWAIHPGGRAILDHIEGVLGLDDSKLAASRHVLSEFGNMSGTTVIFVLDELRRRRAAMAMQGGEAPEWGVMMAFGPGITIETMVLHAPSNLDLKGN

SEQ ID NO: 5 - protein, 405 aa, Unknown
  polyketide synthase
MGSMGKALPATVDEIRRAQRAEGPAAVLAIGTANPPTIMPQDDYPDYYFRVTNSEHLTDLKAKLSRICNHNKSGIRQRYLHLNEELLAANPGFIDPKRPSLDERVEMASAAVPELAAKAATKAIAEWGRPATDITHLIFSTYSGARAPSGDRRLASLLGLRPTVSRTILNLHGCYGGGRSLQLAKEIAENNRGARVLVACSELTLIAFYGPEGGCVDNIIGQTLFGDGAGAVVVGADPDAAVERPLFEMAFATQTTIPESEDAISMQYSKCGMEYHLSSKVPRLIGCNVERSLVDTFRTLGVTAAWNDLFWAVHPGGRAILDNIEEVLGLEDDKLAASRHVLSEFGNMSGTTVIFVLDELRRRRAAAAKQGGETPEWGVLMAFGPGITIETIVLHTPSNPELEGN

SEQ ID NO: 6 - protein, 400 aa, Unknown
  polyketide synthase
MGSAPATIGDMRRAQRADGPAAVLGIGTANPPTCLAQDEYPDYYFRVTKSEHLTDLKGKLTRICNKSGIKQRFIHLDEQLLAANPDFTDRTLPSLDARVEIASAAVPELAASAAAKAIADWGRPATDITHLIFSTYSGARAPSADRRLASLLGLSPTVSRTILNLHGCYGGGRSLQLAKELAENNRGARVLVACSEITLIAFYGPEGGCPDNILGQALFGDGAGAVIVGADPVSPVERPLFEMAFASQTTIPETEDAISMQINKGGMEYHISNQVPRLLGCNVERCLVDAFGALGINNNDWNDLFWAIHPGGRAILDHIEGVLGLDDGKLAASRHVLSEFGNMSGTTVIFVLDELRRRRGLAVKQEEGKAPEWGVMMAFGPGITIETMVLRAPAANLEGN

SEQ ID NO: 7 - protein, 396 aa, Unknown
  polyketide synthase
MGSAPATVVGEIRRAQRADGPAAVLGIGTANPPTSMAQDEYPDYYFRVTNSEHLTDLKAKLTRICKKSGIKQRFMHLNEDLLAANPDFTDRTLPSLDARVDIASAAVPELAAAAAAKAITEWGRPATEITHLIFSTYSGARAPSADRRLASLLGLSPTVSRTMLNLHGCYGGGRSLQLAKELAENNRGARVLVACSEITLIAFYGPEGGCPDNILGQSLFGDGAGAVIIGADPVGPVERPLFEMAFASQTTIPGTEDDISMEINKGGMEYHISNKVPRLLGCNVERCLIDAFGALGVSAKWNDLFWAIHPGGRAILDHIEGVLGLDDGKLAASRHVLSEFGNMSGTTVIFVLDELRHRRVAKLDGEAPEWGVMMAFGPGITIETMVLHAPASLEGN

SEQ ID NO: 8 - protein, 397 aa, Unknown
  polyketide synthase
MGSAPAATAGEIRRAQRADGPAAVLAIGTANPPTSMTQDEYPDYYFRVTNSEHLTDLKAKLTRICKKSGIKQRFMHLNEELLAANPDFTDRTLPSLDARVDIASAAVPELAAAAAAKAIAEWGRPATEITHLIFSTYSGARAPSADRRLASLLGLSPTVSRTMLNLHGCYGGGRSLQLAKELAENNRGARVLVACSEITLIAFYGPEGGCPDNILGQALFGDGAGAVIIGADPVGPVERPLFEMAFASQTTIPGTEDDISMEINKGGMEYHISNKVPRLLGCNVERCLIDAFGALGVSAKWNDLFWAIHPGGRAILDHIEGVLGLDDGKLAASRHVLSEFGNMSGTTVIFVLDELRRRRATKQEGVEAPEWGVMMAFGPGITIETMVLHAPAILDEN

SEQ ID NO: 9 - protein, 101 aa, Unknown
  polyketide synthase
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 10 - protein, 115 aa, Unknown
  PKC4 (polyketide cyclase)
MGEANKGVVKHVFILKMKEGLSNDQIEQMNKDYANLVNLVPSMKALQWGKLEVNNKLGNGGYTHIFESTFESMEGVAEYADHPAHLHLRNLYFHTLDKFLVFDYKPTIVLPNSSY

SEQ ID NO: 11 - protein, 115 aa, Artificial
  chimeric polyketide cyclase
MGEANKGVVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 12 - protein, 101 aa, Unknown
  polyketide cyclase
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 13 - protein, 33 aa, Artificial
  F1 linker
GGGGSGGGGSAEAAAKAEAAAKAGGGGSGGGGS

SEQ ID NO: 14 - protein, 29 aa, Artificial
  F2 linker
GGAEAAAKEAAAKAGGSGGGSGGGGSGGS

SEQ ID NO: 15 - protein, 39 aa, Artificial
  F3 linker
GGAEAAAKEAAAKAAEAAAKEAAAKAGGGSPGPGPGGGS

SEQ ID NO: 16 - protein, 36 aa, Artificial
  F4 linker
GSSSSSSGSSSSSSGSSSSSSGSSSSSSGSSSSSSG

SEQ ID NO: 17 - protein, 15 aa, Artificial
  F5 linker
GGGGSGGGGSGGGGS

SEQ ID NO: 18 - protein, 19 aa, Artificial
  F6 linker
GGEAAAKEAAAKEAAAKGG

SEQ ID NO: 19 - protein, 21 aa, Artificial
  F7 linker
GGAEAAAKEAAAKAPAPAPAG

SEQ ID NO: 20 - protein, 11 aa, Artificial
  F8 linker
GTPTPTPTPTG

SEQ ID NO: 21 - protein, 30 aa, Artificial
  F9 linker
GGGGSGGGGSGGGGSGGGGSGGGGSGGGGS

SEQ ID NO: 22 - protein, 52 aa, Artificial
  F10 link
GGAEAAAKEAAAKAAEAAAKEAAAKAAEAAAKEAAAKAAEAAAKEAAAKAGG

SEQ ID NO: 23 - protein, 39 aa, Artificial
  F11 linker
GGAEAAAKEAAAKAGGSGGGSGGGGSGGSGGGGSGGGGS

SEQ ID NO: 24 - protein, 10 aa, Artificial
  F12 linker
GGGGSGGGGS

SEQ ID NO: 25 - protein, 17 aa, Artificial
  F13 linker
GGSGSAGSAAGSGEFGG

SEQ ID NO: 26 - protein, 33 aa, Artificial
  F14 linker
GGAEAAAKEAAAKAPAPAPAEAAAKEAAAKAGG

SEQ ID NO: 27 - protein, 22 aa, Artificial
  F15 linker
GGSGGAEAAAKEAAAKAGGSGG

SEQ ID NO: 28 - protein, 660 aa, Unknown
  HCS2 (hexanoyl-CoA synthetase)
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 29 - protein, 634 aa, Unknown
  HCS3 (hexanoyl-CoA synthetase)
MSKDTSVLLEEKRVFIPHYTVVEEAHIKNWEAELEKGKDHENYWAEKAERFEWFRKWDRVLDESNRPFYRWFVNGKINMTYNAVDRWLDTDKRNQVAILYVNERGDERKLTYYELYREVNRTANALKSLGIKKGDAVALYLPMCPELVISMLACAKIGAVHSVTYSGLSVGALVERLNDARAKIIITADGTYRRGGVIKLKPIVDEAILQCPTIETTVVVKHTDIDIEMSDISGREMLFDKLIEGEGDRCDAEEMDAEDPLFILYTSGSTGKPKGVLHTTGGYMVGVASTLEMTFDIHNGDLWWCTADIGWITGHSYVVYGPLLLGTTTLLYEGAPDYPDPGVWWSIVEKYGVTKFYTAPTAIRHLMRFGDKHPKRYNLESLKILGTVGGPINPEAWMWYYRNIGREKCPIIDTWGQIETGMHLIAPLPVTPLKPGSVTKPLPGIEADVVDENGDPVPLGKGGFLVIRKPWPAMFRTLFNDEQRYIDVYWKQIPGGVYTAGDMARKDEDGYFWIQGRSDDVLNIAGHRIGTAEVESVFVAHPAVAEAAVIGKADPIKGEVIKAFLILKKGHKLNAALIEELKRHLRHELGPVAVVGEMVQVDSLPKTRSGKIMRRILRAKEEGEDLGDTSTLEE

SEQ ID NO: 30 - protein, 538 aa, Unknown
  HCS4 (hexanoyl-CoA synthetase)
MWNDHDSPEEFNFASDVLDYWAQMEEEGKRGPSPAFWWVNGQGDEIKWSFRKLRDLTCRTANVFEQICGLQQGDHLALILPRVPEWWLVTVGCMRTGIIFMPGTTQLKAKDILYRIQISRAKAIVTTASLVPEVESVASECPDLKTKLVVSDHSHEGWLDFCSLIKSASPDHTCIKSKMKDPMAIFFTSGTTGYPKMAKHNQGLAFRSYIPSCRKLLKLKTSDILWCMSDPGWILATVGCLIEPWTSGCTVFIHHLPQFDPKVIVEVLFKYPITQCLAAPGVYRMVLQQKTSNLRFPTLEHCTTGGESLLPEEYEQWKQRTGLSIHEVYGQSETGISSATLREMKIKRGSIGKAILPFDLQIIDEKGNIPPNTEGYIGIRIKPTRPLGLFMEYENSPESTSEVECGDFYNSGDRATIDEEGYIWFLGRGDDVINASGYRIGPVEVENALAEHPAVAESAVVSSPDKDRGEVVKAFIVLNPEFLSHDQEQLIKELQHHVKSVTAPYKYPRKVEFVSELPKTVTGKIKRKELRNKEFGQL

SEQ ID NO: 31 - protein, 606 aa, Unknown
  HCS5 (hexanoyl-CoA synthetase)
MNTKKKFTSLIHLLCYRGTNKPNQKAYTFIGNGKTETASLTYGELEKRSRAIAAQLQEMGVTRGERALLLYSQPLDFICAFFGCLYAGVIAIPAPPPDAIRLKRTLPRLQACVKDAQVSLVLTTSQIYSQFPSEWQKDFEYNNMLLWLFTEEISEQLANQWQELKINLDAIAYLQYTSGSTSTPKGVIVTHNNVMHHSAYIKQAWNYTSDSIAATWMPYFHDYGLIDGLIQPIYSGITCYVMSPLTFVRRPTCWLEVISKYKVTHSQSPNFGYDYCVRQVTSEQLNNLDLRSWKTASNGAEPIRKDTIEKFIKTFEPCGFRATAFFPSYGLAEATLLVATKSHNDVPEIASIAASALEKNQIVECDGNQKGTRYVVSCGFPICGIKVIIINPNTLTRCASDEVGEIWVSDLSVAQGYWNRPEETKHTFEAYLADTGEGPFLRTGDLGFIKNGQLFVTGRLKDVIIIRGQNHYPQDLEFTVEQSHPALRKNSEAAFGIEVDGEEKLVVVQEVERSWLRKLDFDQVNGDIRQALMEQHELQVYAIALIKPGSIPKTSSGKIMRHACRIKFLEGTLEVINSGGGNPEHLRRLTVMSVMRATPKNGAHLG

SEQ ID NO: 32 - protein, 545 aa, Unknown
  HCS6 (hexanoyl-CoA synthetase)
MLGQMMRNQLVIGSLVEHAARYHGAREVVSVETSGEVTRSCWKEVELRARKLASALGKMGLTPSDRCATIAWNNIRHLEVYYAVSGAGMVCHTINPRLFIEQITYVINHAEDKVVLLDDTFLPIIAEIHGSLPKVKAFVLMAHNNSNASAQMPGLIAYEDLIGQGDDNYIWPDVDENEASSLCYTSGTTGNPKGVLYSHRSTVLHSMTTAMPDTLNLSARDTILPVVPMFHVNAWGTPYSAAMVGAKLVLPGPALDGASLSKLIASEGVSIALGVPVVWQGLLAAQAGNGSKSQSLTRVVVGGSACPASMIREFNDIYGVEVIHAWGMTELSPFGTANTPLAHHVDLSPDEKLSLRKSQGRPPYGVELKIVNDEGIRLPEDGRSKGNLMARGHWVIKDYFHSDPGSTLSDGWFSTGDVATIDSDGFMTICDRAKDIIKSGGEWISTVELESIAIAHPHIVDAAVIAARHEKWDERPLLIAVKSPNSELTSGEVCNYFADKVARWQIPDAAIFVEELPRNGTGKILKNRLREKYGDILLRSSSSVC

SEQ ID NO: 33 - protein, 548 aa, Unknown
  HCS7 (hexanoyl-CoA synthetase)
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 34 - protein, 525 aa, Artificial
  PKC1.1-F3-PKS1 polyketide cyclase polyketide synthase fusion protein
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAAEAAAKEAAAKAGGGSPGPGPGGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 35 - protein, 515 aa, Artificial
  PKC1.1-F2-PKS1 polyketide cyclase polyketide synthase fusion protein
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 36 - protein, 515 aa, Artificial
  PKS1-F2-PKC1.1 polyketide synthase polyketide cyclase fusion protein
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYGGAEAAAKEAAAKAGGSGGGSGGGGSGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 37 - protein, 519 aa, Artificial
  PKS1-F1-PKC1.1 polyketide synthase polyketide cyclase fusion protein
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYGGGGSGGGGSAEAAAKAEAAAKAGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 38 - protein, 501 aa, Artificial
  PKS1-F5-PKC1.1 polyketide synthase polyketide cyclase fusion protein
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYGGGGSGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 39 - protein, 522 aa, Artificial
  PKS1-F4-PKC1.1 polyketide synthase polyketide cyclase fusion protein
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYGSSSSSSGSSSSSSGSSSSSSGSSSSSSGSSSSSSGMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 40 - protein, 174 aa, Artificial
  PKC1_Zip_1N1C, polyketide cyclase comprising a C-terminal and N-terminal small “Zip” peptide that facilitates dimerization of the enzyme
MSPEDENRELEEKIRELKEKNEELKREIKYLEEAVKHLIVLKFKDEITEAQKEEFFKTYVNLVNIIPAGGGGSMKDVYWGKDVTQKNKEEGYTHIVEVTFESVETIQDYIIHPAHVGFGDVYRSFWEKLLIFDYTPRKGGGGSPEDKNEELKREIERLEEENRELERKIEYLKR

SEQ ID NO: 41 - protein, 167 aa, Artificial
  PKC1_Zip_1N2C, polyketide cyclase comprising a C-terminal and N-terminal small “zip” peptide that facilitates dimerization of the enzyme
MSPEDENRELEEKIRELKEKNEELKREIKYLEEAVKHLIVLKFKDEITEAQKEEFFKTYVNLVNIIPAGSMKDVYWGKDVTQKNKEEGYTHIVEVTFESVETIQDYIIHPAHVGFGDVYRSFWEKLLIFDYTPRKGPEDKNEELKREIERLEEENRELERKIEYLKR

SEQ ID NO: 42 - protein, 174 aa, Artificial
  PKC4.8_Zip_1N2D, chimeric polyketide cyclase comprising C-terminal and N-terminal small “Zip” peptide that facilitates dimerization of the enzyme
MSPEDENRELEEKIRELKEKNEELKREIKYLEEGVVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPTIVLPNSSYSPEDKNEELKREIERLEEENRELERKIEYLKR

SEQ ID NO: 43 - protein, 176 aa, Artificial
  PKC1.1_Ub_1, polyketide cyclase with ubiquitin attached at N-terminal end
MQIFVKTLTGKTITLEVESSDTIDNVKSKIQDKEGIPPDQQRLIFAGKQLEDGRTLSDYNIQKESTLHLVLRLRGGAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 44 - protein, 167 aa, Artificial
  P3-PKC1.1, polyketide cyclase that comprises a scaffolding tag P3
MGSSPEDEIQQLEEEIAQLEQKNAALKEKNQALKYGSGGAEAAAKEAAAKAGGSGGGSGGGGSGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 45 - protein, 167 aa, Artificial
  PKC1.1-P4, polyketide cyclase that comprises a scaffolding tag P4
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGSPEDKIAQLKQKIQALKQENQQLEEENAALEYGGSG

SEQ ID NO: 46 - protein, 581 aa, Artificial
  P3-PKC1.1-PKS1, polyketide cyclase polyketide synthase fusion protein that comprises a scaffolding tag P3
MGSSPEDEIQQLEEEIAQLEQKNAALKEKNQALKYGSGGAEAAAKEAAAKAGGSGGGSGGGGSGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 47 - protein, 33 aa, Artificial
  Zip_1N, N-terminal sequence small “Zip” peptide linker 1
MSPEDENRELEEKIRELKEKNEELKREIKYLEE

SEQ ID NO: 48 - protein, 36 aa, Artificial
  Zip_1C, C-terminal sequence small “Zip” peptide linker 1
GGGGSPEDKNEELKREIERLEEENRELERKIEYLKR

SEQ ID NO: 49 - protein, 32 aa, Artificial
  Zip_2C, C-terminal sequence small “Zip” peptide linker 2
GPEDKNEELKREIERLEEENRELERKIEYLKR

SEQ ID NO: 50 - protein, 214 aa, Unknown
  CHIL1 (chalcone isomerase-like protein)
MATSDGSSNAATKEEAVQVEPKTGISFPVKLDDGKILYCVGYNKKSLLGLSIKAYGFGLYVDSDKLKDVLKSKIEKAPSKPTEEMYQLAIDGDFGMTIKMVVSFSGVKLSMAKKGFTEAMRESMKKLTGQKNEELSNKVFGTTSDKIKLRLGSEMIVSKLPGYVLETKVNGELVSRVESELLCRAYFRNYLGEDTLECEKESREMFGQSMLSLF

SEQ ID NO: 51 - protein, 209 aa, Unknown
  CHIL2 (chalcone isomerase-like protein)
MANNMVMVHEIPFPTEIKTTKPLSLLGYGITDMEIHFLQIKFTAIGVYLDPDVVKHVQQWKGKKGNELAEDDDFFDALISAPVEKYLRIVVIKEIKGSQYGVQLESSVRDRLAADDKYEEEEEAALEKIVEFFQSKYFKKDTLITFHFPATSPTAEIVVTIEGKEEFKLDVENENVVEMIKKWYLGGTTGASPSTISSLADNLSAQLSK

SEQ ID NO: 52 - protein, 214 aa, Unknown
  CHIL3 (chalcone isomerase-like protein)
MATALNSKNASSNTAVHIEPKTGIAFPVKLDDGKSLNSVGLRKKSLLGMGIKVFGFGLYADNEKLKNLLKLKIGKSPAKPTEEMYQLVIDGDIGLTHKIVIAYSGLKMNMFKKAFSEALGESIMKLNGGRKNEELANKVLGPASDQIKLATGSEMEISKLPGYVLETKVHGELASRVESELLCRAYFGIYLGEITMECYKESKEMFGQSMLSLF

SEQ ID NO: 53 - protein, 209 aa, Unknown
  CHIL4 (chalcone isomerase-like protein)
MENNMVMVHEIPFPPEIKTTKPLSLLGYGITDMEIHFLQVKFTAIGVYLDSDVVKHLQQWKGKKGNELAEDDDFFDALISAPVEKYLRIVVIKEIKGSQYGVQLESSVRDRLAAEDMYEEEEEAALEKIVEFLQSKYFKKDTLITFHFPATSPTAEIVVTLEGKEESKLKVENKNVVDMIKKWYLGGTSGVSPSTISSLADNLSAELSK

SEQ ID NO: 54 - protein, 231 aa, Unknown
  CHIL5 (chalcone isomerase-like protein)
MAVPEVVVEGVVFPPVARPPGSAGSHFLGGAGVRGIEIGGNFIKFTAIGVYLEDAAVSALAKKWAGKTADELASDAAFFRDVVTGDFEKFTRVTMLLPLTGEQYAGKVTENCVAFWKAVGLYTDAEGVAVEKFKEAFKPETFPPGASILFTHSSTGVLTVAFSKDSSVPASGGVAIENKHLCEAVLESIIGEHGVSPAAKLSLAARVSELLTKGTAGAADAPQAEPVSVTA

SEQ ID NO: 55 - protein, 222 aa, Artificial
  CHIL6 (chalcone isomerase-like protein)
MLISAVGSETKTITFEGIPFPAEITAAGNPLSLLATGITDIEIHFLQIKYNAIGVYLHSNDDSDLLTTHLGAWKGKTAEDLLADAAFWSALVSSPVEKLLRVVVIKEIKGSQYGVQLESSVRDRLAAVDLYEDDEEEALEKVAEFFQAKYFKPGSVITFHFPATPGPADITFVTEGKADAKITVENEHVAGMIQKWYLGGDNAVSPTTVRSLADRFAALLAA

SEQ ID NO: 56 - protein, 212 aa, Unknown
  CHIL7 (chalcone isomerase-like protein)
MGTEMATVEVEGIPFPQEITRTKPLSFLAHGVTDIEIHFLQIKYNAIGVYLDKESVLGHLESWKGKKAEELVQDAGFFQALVFAPVEKLFRIVVIKEIKGSQYGVQLESSVRDRLVAVDKFEEEEEEALEKVTEFFQYKYFKPNSVLTFHFPTTPGIAEISFVTEGKSEAKLTVDNNNVAEMIQKWYLGGESAVSPTTVKSLADQFAPLLSA

SEQ ID NO: 57 - protein, 208 aa, Unknown
  CHIL8 (chalcone isomerase-like protein)
MATVEVEGIPFPQEITVSKPLSLLAHGITDIEIHFLQIKYNAIGVYLEKDNVLGHLESWKGKKAEELVQDDGFFQALVSAPVEKLFRIVVIKEIKGSQYGVQLESSVRDRLVSVDKYEDEEEESLEKVTEFFQSKYFKPNSVLTFHFPNTPGIAEISFVTEGKGEAKLTVENKNVAEMIQKWYLGGESAVSPTTVKSLADQFAALLSA

SEQ ID NO: 58 - protein, 190 aa, Artificial
  PKC4_Ub_1, polyketide cyclase with ubiquitin attached at N-terminal end
MQIFVKTLTGKTITLEVESSDTIDNVKSKIQDKEGIPPDQQRLIFAGKQLEDGRTLSDYNIQKESTLHLVLRLRGGGEANKGVVKHVFILKMKEGLSNDQIEQMNKDYANLVNLVPSMKALQWGKLEVNNKLGNGGYTHIFESTFESMEGVAEYADHPAHLHLRNLYFHTLDKFLVFDYKPTIVLPNSSY

SEQ ID NO: 59 - protein, 190 aa, Artificial
  PKC4.8_Ub_1, chimeric polyketide cyclase with ubiquitin attached at N-terminal end
MQIFVKTLTGKTITLEVESSDTIDNVKSKIQDKEGIPPDQQRLIFAGKQLEDGRTLSDYNIQKESTLHLVLRLRGGGEANKGVVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 60 - protein, 5 aa, Artificial
  F16 linker
GGGGS

SEQ ID NO: 61 - protein, 36 aa, Artificial
  F17 linker
GGGGSAEAAAKAEAAAKAAEAAAKAEAAAKAGGGGS

SEQ ID NO: 62 - protein, 46 aa, Artificial
  F18 linker
GGGGSGGGGSAEAAAKAEAAAKAAEAAAKAEAAAKAGGGGSGGGGS

SEQ ID NO: 63 - protein, 1048 aa, Artificial
  HCS2-F16-PKS1, hexanoyl CoA synthetase polyketide synthase fusion protein
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADGGGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIK

SEQ ID NO: 64 - protein, 1079 aa, Artificial
  HCS2-F17-PKS1, hexanoyl-CoA synthetase polyketide synthase fusion protein
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADGGGGSAEAAAKAEAAAKAAEAAAKAEAAAKAGGGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIK

SEQ ID NO: 65 - protein, 1089 aa, Artificial
  HCS2-F18-PKS1, hexanoyl-CoA synthetase polyketide synthase fusion protein
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADGGGGSGGGGSAEAAAKAEAAAKAAEAAAKAEAAAKAGGGGSGGGGSMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIK

SEQ ID NO: 66 - protein, 37 aa, Artificial
  P3 scaffolding tag
MGSSPEDEIQQLEEEIAQLEQKNAALKEKNQALKYGS

SEQ ID NO: 67 - protein, 37 aa, Artificial
  P4 scaffolding tag
GSPEDKIAQLKQKIQALKQENQQLEEENAALEYGGSG

SEQ ID NO: 68 - protein, 385 aa, Unknown
  PKS1.1 (polyketide synthase)
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSCVLFVMDELRKRSLEEGKSTTGDGFEWGVLFGFGPGLTVERVVVRSVPIKY

SEQ ID NO: 69 - protein, 102 aa, Artificial
  PKC4.11 (engineered polyketide cyclase mutant)
MAVKHLIVLKFKDEITEAQKEEFFKTYVNLVNIIPAMKDVYWGKLEVNNKLGNGGYTHIVEVTFESVETIQDYIIHPAHVGFGDVYRSFWEKLLIFDYTPRK

SEQ ID NO: 70 - protein, 114 aa, Artificial
  PKC4.15 (engineered polyketide cyclase mutant)
MGEANKGVVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPTIVLPNSSY

SEQ ID NO: 71 - protein, 115 aa, Artificial
  PKC4.17 (engineered polyketide cyclase mutant)
MGEANKGVVKHLIVLKFKDEITEAQKEEFFKTYVNLVNIIPAMKDVYWGKLEVNNKLGNGGYTHIVEVTFESVETIQDYIIHPAHVGFGDVYRSFWEKLLIFDYTPTIVLPNSSY

SEQ ID NO: 72 - protein, 102 aa, Artificial
  PKC4.19 (engineered polyketide cyclase mutant)
MAVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPRK

SEQ ID NO: 73 - protein, 109 aa, Artificial
  PKC4.30 (engineered polyketide cyclase mutant)
MAVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 74 - protein, 108 aa, Artificial
  PKC4.31 (engineered polyketide cyclase mutant)
MGEANKGVVKHVIILKFKEGITEAQKEEFFKTYVNLVNLVPAMKAVQWGKLEVNNKLGNGGYTHIVESTFESVETIQDYIIHPAHVGFGDVYRSFWEKLLIFDYTPRK

SEQ ID NO: 75 - protein, 174 aa, Artificial; identical to SEQ ID NO: 42
  PKC4.32 (engineered polyketide cyclase mutant)

SEQ ID NO: 76 - protein, 115 aa, Artificial
  PKC4.33 (engineered polyketide cyclase mutant)
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 77 - protein, 109 aa, Artificial
  PKC4.35 (engineered polyketide cyclase mutant)
MAVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 78 - protein, 108 aa, Artificial
  PKC4.36 (engineered polyketide cyclase mutant)
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPRK

SEQ ID NO: 79 - protein, 102 aa, Artificial
  PKC4.37 (engineered polyketide cyclase mutant)
MAVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPRK

SEQ ID NO: 80 - protein, 174 aa, Artificial
  PKC4.38 (engineered polyketide cyclase mutant)
MSPEDENRELEEKIRELKEKNEELKREIKYLEEGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYSPEDKNEELKREIERLEEENRELERKIEYLKR

(19) United States
(12) Patent Application Publication
(10) Pub. No.: US 2024/0360425 A1
(43) Pub. Date: Oct. 31, 2024

(54) ENGINEERED ENZYMES, CELLS, AND METHODS FOR PRODUCING CANNABINOID PRECURSORS AND CANNABINOIDS

(71) Applicant: Cellibre, Inc., San Diego, CA (US)

(72) Inventors: Spiros Kambourakis, San Diego, CA (US); Nicholas Donald Keul, San Diego, CA (US); Russell Scott Komor, San Diego, CA (US); Jun Urano, Irvine, CA (US); Nicky Christopher Caiazza, Encinitas, CA (US)

(21) Appl. No.: 18/290,561

(22) PCT Filed: May 13, 2022
(86) PCT No.: PCT/US2022/029327

§ 371 Date: Nov. 14, 2023

Related U.S. Application Data
(60) Provisional application No. 63/188,645, filed on May 14, 2021.

(51) Int. Cl. C12N 9/10; C12P 7/26

(52) U.S. Cl. CPC C12N 9/1029; C12P 7/26; C12Y 203/01086

(57) ABSTRACT

Described herein are the discovery and/or optimization of enzymes involved in the biosynthesis of olivetolic acid, divarinic acid, and analogs thereof.

### CROSS-REFERENCE TO RELATED APPLICATIONS

This application is a U.S. national stage filing under 35 U.S.C. 371 of International Application No. PCT/US2022/029327, filed May 13, 2022, which claims the benefit of U.S. Provisional Application No. 63/188,645, filed May 14, 2021. The entire teachings of the above applications are hereby incorporated by reference in their entirety. International Application No. PCT/US2022/029327 was published under PCT Article 21(2) in English.

### BACKGROUND OF THE INVENTION

The Cannabaceae family of plants produces numerous different cannabinoids (>=120) in variable, relative quantities over a 7-10 week flowering period. Many of these cannabinoids have been and are currently being explored as therapeutics in chordates (e.g., mammals), and as a result, they are largely approved for medical and/or recreational use in the United States (Abrams D I Eur J Int Med 2018, 49, 7-11). Specifically, the most sought after (phyto)cannabinoids are (i) tetrahydrocannabinolic acid (THCA); (ii) cannabidiolic acid (CBDA), and cannabichromenic acid (CBCA). These phytocannabinoids as well as their associated chemical analogs (e.g., THCVA, CBDVA, and CBCVA) are all biosynthesized in various quantities from the same pre-cursor in the cannabinoid biosynthetic pathway, which is cannabigerolic acid and cannabigerovarinic acid (i.e., CBGA and CBGVA, respectively). Thus, to mass produce any specific phytocannabinoid (e.g., THC(V)A/CBD(V)A/CBC(V)A/etc), both the rate and total quantity of biosynthesized CBG(V)A must be increased. The biosynthesis of these hydrophobic compounds and their on-pathway intermediates creates limitations with regards to production. Specifically, cells are limited by the following on-pathway pre-cursor molecules: (i) the amount of available pre-cursor molecules fluxing through the pathway to the terminal phytocannabinoids, such as C3-CoA/C4-CoA/C6-CoA, OA/DVA, and CBGA/CBGVA. Further limitations to mass producing these phytocannabinoids is the intracellular availability of available geranyl pyrophosphate (GPP) and the pre-cursors that lead to the native synthesis of this compound (e.g., MEV pathway). To sustainably meet the demands of both the consumer and medicinal market for these valuable terminal phytocannabinoids (e.g., THC(V)A, CBD(V)A, CBC(V)A) there is a need for the scaled production of cannabinoid biosynthesis in non-native hosts. Thus, the total production capacity of cannabinoids is accelerated by the engineering of enzymes in the cannabinoid biosynthesis pathway within a non-native host.

### SUMMARY OF THE INVENTION

Described herein is the discovery and/or optimization of enzymes involved in the biosynthesis of Olivetolic acid (OA) from hexanoic acid. Specifically, disclosed are engineered enzymes, cells, and methods that significantly enhance both the rate and total quantity at which cannabinoid precursors can be biosynthesized. This is accomplished by increasing the flux of required precursors for CBGAS activity, olivetolic acid (OA) and geranyl pyrophosphate (GPP) (FIG. 1). The mevalonic acid pathway (MVA) converts acetyl-CoA to geranyl pyrophosphate (GPP) and the olivetolic acid/hexanoic acid pathway converts acetyl-CoA to olivetolic acid (OA) through the intermediacy of hexanoyl-CoA (FIG. 1). The rate and efficiency (flux) of the intracellular formation of CBGA is one critical factor that defines the final titers of the most common cannabinoids (THCA, CBDA, and CBCA) because they are all synthesized from CBGA by the action of three different synthases (THCA, CBDA and CBCA synthase, respectively). Key to achieving high THC(A), CBD(A) and CBC(A) titers either in the plant or in a recombinant host organism is sufficient flux and availability of CBGA precursors, namely GPP and OA. The optimization of OA biosynthesis from hexanoic acid is described herein. These methods were also used to produce a large number of OA analogs, including DVA, as shown in FIG. 2. Further, novel enzymes for OA synthesis (and its analogs) were identified and/or improved by engineering.

HCS: Hexanoyl-CoA synthetases: a variety of natural enzymes were found to catalyze hexanoyl-CoA and butyryl-CoA from hexanoic acid and butyric acid, respectively.

PKS: polyketide synthase (Type III): natural enzymes were identified, and their activity improved via protein engineering

PKC: polyketide cyclase: new, nonnaturally occurring enzymes were developed and improved by engineering.

Fusions of PKS & PKC: showed improved OA titer and OA/OL ratio.

Fusions of HCS & PKS: may increase the flux to tetraketide (and ultimately to OA and analogs) from carboxylic acid

CHIL proteins: may increase enzyme's activity and reduce byproduct formation of HTAL and PDAL.

Some aspects of the present disclosure are directed to a polyketide synthase comprising an amino acid sequence with at least 70% identity to SEQ ID NO: 2, 3, 4, 5, 6, 7, 8, or 68 wherein the polyketide synthase has polyketide synthase (PKS) activity. In some embodiments, the amino acid sequence of the polyketide synthase comprises at least one amino acid modification as compared to SEQ ID NO: 2, 3, 4, 5, 6, 7, 8, or 68. In some embodiments, the polyketide synthase comprises an amino acid sequence with at least one amino acid substitution as compared to SEQ ID NO: 2, wherein the amino acid substitution is located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least one amino acid substitution as compared to SEQ ID NO: 3, wherein the amino acid substitution is located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least one amino acid substitution as compared to SEQ ID NO: 4, wherein the amino acid substitution is located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least one amino acid substitution as compared to SEQ ID NO: 5, wherein the amino acid substitution is located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least one amino acid substitution as compared to SEQ ID NO: 6, wherein the amino acid substitution is located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least one amino acid substitution as compared to SEQ ID NO: 7, wherein the amino acid substitution is located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least one amino acid substitution as compared to SEQ ID NO: 8, wherein the amino acid substitution is located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least one amino acid substitution as compared to SEQ ID NO: 68, wherein the amino acid substitution is located in SEQ ID NO: 68 at positions selected from S126, G156, C157, G193, G204, F208, G209, D210, I248, H297, G299, N330, S332, F367, G368, and P369. In some embodiments, the polyketide synthase further comprises a cleavage sequence, a linker sequence, a solubility tag, scaffolding tag, dimer-association small peptide extension off the termini, or affinity tag sequence.

In some embodiments, the polyketide synthase produces tetraketides with a variety of alkyl chain lengths from the condensation of one or more acyl-CoA substrates, all with varying alkyl chain lengths. In some embodiments, the tetraketides, are condensed from one or more acyl-CoA substrates selected from the group consisting of Acetyl-CoA, Butyryl-CoA, Hexanoyl-CoA, Octanoyl-CoA, Decanoyl-CoA, Dodecanoyl-CoA, Myristoyl-CoA, Palmitoleyl-CoA, Linoleyl-CoA, Palmityl-CoA, Malonyl-CoA, and Oleyl-CoA. In some embodiments, the polyketide synthase comprises a polypeptide sequence with at least 70% identity with SEQ ID NO: 2, 3, 4, 5, 6, 7, 8, or 68 and produces a corresponding di-, tri-, or tetraketide of various alkyl chain-lengths from one or more acyl-CoA substrates shown in FIG. 2. In some embodiments, the polyketide synthase produces the tetraketide from the acyl-CoA substrate at a higher rate than the native PKS1 from Cannabis sativa.

Some aspects of the present disclosure are directed to a cell comprising the polyketide synthase disclosed herein. In some embodiments, the cell is a bacteria cell or a yeast cell.

Some aspects of the present disclosure are directed to a polynucleotide coding for a polyketide synthase described herein.

Some aspects of the present disclosure are directed to a polyketide cyclase comprising an amino acid sequence with at least 70% identity to SEQ ID NO: 9, 10, 11, 12, 69, 71, 72, 73, 74, 75, 76, 77, 78, 79 or 80, wherein the polyketide cyclase has polyketide cyclase (PKC) activity. In some embodiments, the amino acid sequence of the polyketide cyclase comprises at least one amino acid modification as compared to SEQ ID NO: 9, 10, 11, 12, 69, 70, 71, 72, 73, 74, 75, 76, 77, 78, 79, or 80.

In some embodiments, the amino acid sequence of the polyketide cyclase comprises a chimeric amino acid sequence comprising portions of two or more of SEQ ID NOs 9, 10, 11, 12, 69, 70, 71, 72, 73, 74, 75, 76, 77, 78, 79, and/or 80. In some embodiments, the amino acid sequence of the polyketide cyclase comprises a chimeric amino acid sequence comprising portions of SEQ ID NO: 9 and SEQ ID NO: 10, portions of SEQ ID NO: 71 and SEQ ID NO: 72, portions of SEQ ID NO: 76 and SEQ ID NO: 72, portions of SEQ ID NO: 69 and SEQ ID NO: 71 or portions of SEQ ID NO: 69 and SEQ ID NO: 76.

In some embodiments, the polyketide cyclase comprises an amino acid sequence with at least one amino acid modification as compared to SEQ ID NO: 10, wherein the amino acid substitution is located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with a 1-20 C-terminus or N-terminus truncation as compared to SEQ ID NO: 10.

In some embodiments, the polyketide cyclase comprises an amino acid sequence with at least one amino acid substitution as compared to SEQ ID NO: 11, wherein the amino acid substitution is located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with a 1-20 C-terminus or N-terminus truncation as compared to SEQ ID NO: 11. In some embodiments, the polyketide cyclase further comprises a cleavage sequence, a linker sequence, a solubility tag, a scaffolding tag, dimer-promoting small peptide terminal extension, or affinity tag sequence.

In some embodiments, the polyketide cyclase comprises an amino acid sequence with at least one amino acid substitution as compared to SEQ ID NO: 69, wherein the amino acid substitution is located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with a 1-20 C-terminus or N-terminus truncation as compared to SEQ ID NO: 69. In some embodiments, the polyketide cyclase further comprises a cleavage sequence, a linker sequence, a solubility tag, a scaffolding tag, dimer-promoting small peptide terminal extension, or affinity tag sequence.

In some embodiments, the polyketide cyclase comprises an amino acid sequence with at least one amino acid substitution as compared to SEQ ID NO: 71, wherein the amino acid substitution is located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with a 1-20 C-terminus or N-terminus truncation as compared to SEQ ID NO: 71. In some embodiments, the polyketide cyclase further comprises a cleavage sequence, a linker sequence, a solubility tag, a scaffolding tag, dimer-promoting small peptide terminal extension, or affinity tag sequence.

In some embodiments, the polyketide cyclase comprises an amino acid sequence with at least one amino acid substitution as compared to SEQ ID NO: 72, wherein the amino acid substitution is located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with a 1-20 C-terminus or N-terminus truncation as compared to SEQ ID NO: 72. In some embodiments, the polyketide cyclase further comprises a cleavage sequence, a linker sequence, a solubility tag, a scaffolding tag, dimer-promoting small peptide terminal extension, or affinity tag sequence.

In some embodiments, the polyketide cyclase comprises an amino acid sequence with at least one amino acid substitution as compared to SEQ ID NO: 76, wherein the amino acid substitution is located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with a 1-20 C-terminus or N-terminus truncation as compared to SEQ ID NO: 76. In some embodiments, the polyketide cyclase further comprises a cleavage sequence, a linker sequence, a solubility tag, a scaffolding tag, dimer-promoting small peptide terminal extension, or affinity tag sequence.

In some embodiments, the polyketide cyclase comprises a C-terminal and N-terminal small peptide that can dimerize. In some embodiments, the polyketide cyclase comprises a ubiquitin at the N-terminal. In some embodiments, the polyketide cyclase comprises a C-terminal and/or an N-terminal scaffolding tag capable of forming a homodimer and/or heterodimer.

In some embodiments, the polyketide cyclase is capable of cyclizing the PKS-produced tetraketide to the corresponding 6-alkyl-2,4-dihydroxy benzoic acid as shown in FIG. 2 (Compound B). In some embodiments cyclizing of the tetraketide produces olivetolic acid (OA), an OA chemical analog, divarinic acid (DVA), or a DVA chemical analog from a tetraketide. In some embodiments, the polyketide cyclase is capable of producing OA, an OA analog, DVA, or a DVA analog at a higher rate than PKC4 (SEQ ID NO: 10).

Some aspects of the present disclosure are directed to a cell comprising a polyketide cyclase described herein. In some embodiments, the cell is a bacteria cell or a yeast cell.

Some aspects of the present disclosure are directed to a polynucleotide coding for a polyketide cyclase described herein.

Some aspects of the present disclosure are directed to a fusion protein comprising a polypeptide having polyketide synthase activity and a polypeptide having polyketide cyclase activity. In some embodiments, the polypeptide having polyketide synthase activity comprises an amino acid sequence with at least 70% identity to SEQ ID NO: 1, 2, 3, 4, 5, 6, 7, 8, or 68. In some embodiments, the polypeptide having polyketide cyclase activity comprises an amino acid sequence with at least 70% identity to SEQ ID NO: 9, 10, 11, 12, 69, 70, 71, 72, 73, 74, 75, 76, 77, 78, 79 or 80. In some embodiments, the fusion protein further comprises a linker between the polypeptide having polyketide synthase activity and the polypeptide having polyketide cyclase activity. In some embodiments, the linker is between 5 and 52 amino acids in length. In some embodiments, the linker has an amino acid sequence selected from SEQ ID NO: 13, 14, 15, 16, 17, 18, 19, 20, 21, 22, 23, 24, 25, 26, or 27.

In some embodiments, the fusion protein is a bi-function fusion protein, a tri-functional fusion protein or a tetra-functional fusion protein. In some embodiments, the fusion protein comprises an amino acid sequence having at least 90% identity to SEQ ID NO: 1, 2, 3, 4, 5, 6, 7, 8, or 68 and comprises an amino acid sequence having at least 90% identity to SEQ ID NO: 9, 10, 11, 12, 69, 70, 71, 72, 73, 74, 75, 76, 77, 78, 79, or 80 and forms a bi-functional fusion protein. In some embodiments, the fusion protein comprises the amino acid sequence of SEQ ID NO: 34, 35, 36, 37, or 38.

In some embodiments, the fusion protein is capable of producing olivetolic acid from Hexanoyl-CoA and/or divarinic acid from butyryl-CoA. In some embodiments, the fusion protein is capable of producing a ratio of olivetolic acid to olivetol from Hexanoyl-CoA at a ratio of greater than 0.1.

In some embodiments, the polypeptide having polyketide synthase activity is located at the N-terminus. In some embodiments, the polypeptide having polyketide cyclase activity is located at the N-terminus.

In some embodiments, the fusion protein comprises a C-terminal and N-terminal small peptide that can dimerize. In some embodiments, the fusion protein comprises a ubiquitin at the N-terminal. In some embodiments, the fusion protein comprises a ubiquitin at the C-terminal. In some embodiments, the fusion protein comprises a C-terminal and/or an N-terminal scaffolding tag capable of forming a homodimer and/or heterodimer.

Some aspects of the present disclosure are directed to a cell comprising a fusion protein described herein. In some embodiments, the cell is a bacteria cell or a yeast cell.

Some aspects of the present disclosure are directed to a polynucleotide coding for a fusion protein described herein.

Some aspects of the present disclosure are directed to a cell comprising an exogenous nucleotide sequence coding for at least one of the following: (a) a polyketide synthase comprising an amino acid sequence with at least 70% identity to SEQ ID NO: 1, 2, 3, 4, 5, 6, 7, 8, or 68, wherein the polyketide synthase has polyketide synthase (PKS) activity; (b) a polyketide cyclase comprising an amino acid sequence with at least 70% identity to SEQ ID NO: 8, 9, 10, 11, 69, 70, 71, 72, 73, 74, 75, 76, 77, 78, 79 or 80, wherein the polyketide cyclase has polyketide cyclase (PKC) activity; (c) a fusion protein comprising a polypeptide having polyketide synthase activity and a polypeptide having polyketide cyclase activity; and (d) an enzyme having acyl-CoA activity.

In some embodiments, the cell comprises (a) the polyketide synthase, wherein the polyketide synthase is capable of producing a tetraketide from acyl-CoA substrates selected from carboxylic acids with two to twenty-two carbons, such as for example Acetyl-CoA, Butyryl-CoA, Hexanoyl-CoA, Octanoyl-CoA, Decanoyl-CoA, Dodecanoyl-CoA, Myristoyl-CoA Palmitoleyl-CoA, Linoleyl-CoA, Palmityl-CoA, and Oleyl-CoA or acyl-CoA substrates shown in FIG. 2. In some embodiments, the cell comprises (b) the polyketide cyclase, wherein the polyketide cyclase is capable of producing olivetolic acid (OA), an OA analog, divarinic acid (DVA), or a DVA analog from a tetraketide. In some embodiments, the cell comprises (c) the fusion protein, wherein the fusion protein is capable of producing olivetolic acid from Hexanoyl-CoA or divarinic acid from Butyryl-CoA. In some embodiments, the cell comprises (d) the enzyme having hexanoyl-CoA synthetase (HCS) activity, wherein the enzyme comprises an amino acid sequence selected from SEQ ID NO: 28, 29, 30, 31, 32, or 33.

In some embodiments, the cell comprises a polyketide synthase as described herein (e.g., an amino acid sequence of SEQ ID NO: 1, 2, 3, 4, 5, 6, 7, 8, or 68) and a polyketide cyclase described herein (e.g., an amino acid sequence of SEQ ID NO: 9, 10, 11, 12, 69, 70, 71, 72, 73, 74, 75, 76, 77, 78, 79, or 80). In some embodiments, the cell comprises a fusion protein of SEQ ID NO: 34, 35, 36, 37, or 38.

Some aspects of the present disclosure are directed to a fusion protein comprising a polypeptide having polyketide synthase activity and a polypeptide having acyl-CoA synthetase activity (e.g., HCS enzymes). In some embodiments, the polypeptide having polyketide synthase activity comprises an amino acid sequence with at least 70% identity to SEQ ID NO: 1, 2, 3, 4, 5, 6, 7, 8, or 68. In some embodiments, the polypeptide having acyl-CoA synthetase activity comprises an amino acid sequence with at least 70% identity to SEQ ID NO: 28, 29, 30, 31, 32 or 33. In some embodiments, the fusion protein further comprises a linker between the polypeptide having polyketide synthase activity and the polypeptide having acyl-CoA synthetase activity. In some embodiments, the linker is between 5 and 52 amino acids in length. In some embodiments, the linker has an amino acid sequence selected from SEQ ID NOs. 60, 61, or 62. In some embodiments, the fusion protein comprises the amino acid sequence of SEQ ID NO: 63, 64, 65

In some embodiments, the fusion protein is capable of producing a tetraketide-CoA from Hexanoic acid and/or Butyric acid.

In some embodiments, the acyl-CoA synthetase peptide is located at the N-terminus of the polyketide synthase peptide. In some embodiments, the acyl-CoA synthetase peptide is located at the C-terminus of the polyketide synthase.

In some embodiments, the cell comprises a acyl-CoA synthetase as described herein (e.g., an amino acid sequence of SEQ ID NO: 28, 29, 30, 31, 32 or 33) and a polyketide synthase as described herein (e.g., an amino acid sequence of SEQ ID NO: 1, 2, 3, 4, 5, 6, 7, 8, or 68) and a polyketide cyclase as described herein (e.g., an amino acid sequence of SEQ ID NO: 9, 10, 11, 12, 69, 70, 71, 72, 73, 74, 75, 76, 77, 78, 79, or 80). In other embodiments the cell comprises a fusion between an acyl-CoA synthetase and a polyketide synthase as described herein (e.g., an amino acid sequence of SEQ ID NO: 63, 64, 65) and a separate polyketide cyclase as described herein (e.g., an amino acid sequence of SEQ ID NO: 9, 10, 11, 12, 69, 70, 71, 72, 73, 74, 75, 76, 77, 78, 79, or 80).

In some embodiments, the cell further comprises an exogenous polynucleotide coding for a CHIL protein. In some embodiments, the cell is capable of utilizing hexanoic acid to produce olivetolic acid. In some embodiments, the cell is capable of utilizing butyric acid to produce divarinic acid. In some embodiments, the cell is capable of utilizing octanoic acid, decanoic acid, dodecanoic acid, oleic acid, palmitic acid, myristic acid or stearic acid to produce one or more olivetolic acid analogs.

In some embodiments, the cell is a bacteria cell or a yeast cell. In some embodiments, the cell is a Yarrowia strain.

Some aspects of the present disclosure are directed to a method of producing one or more 6-alkyl-2,4-dihydroxy benzoic acid(s), e.g., as shown in FIG. 2 (compound B). Some embodiments are directed to contacting the cell with carboxylic acids containing two to twenty-two carbon atoms in conditions suitable to produce a 6-alkyl-2,4-dihydroxy benzoic acid (FIG. 2 compound B). In other aspects, the fatty acid CoAs with carbon chains of 2 to 22 are made in the cell using native or engineered pathways when growing on a carbon source (for example glucose, glycerol or any other sugar). Other aspects of this disclosure are directed to the synthesis of olivetolic acid (OA), an OA analog, divarinic acid (DVA), or a DVA analog comprising contacting a cell described herein with a fatty acid under suitable conditions to produce the olivetolic acid (OA), the OA analog, the divarinic acid (DVA), or the DVA analog.

Some aspects of the present disclosure are directed to a cell comprising a fusion protein as disclosed herein. Some aspects of the present disclosure are directed to a cell comprising a fusion protein as disclosed herein and an enzyme with polyketide cyclase activity. In some embodiments, the enzyme with polyketide cyclase activity is a polyketide cyclase disclosed herein. In some embodiments, the cell is capable of utilizing hexanoic acid to produce olivetolic acid. In some embodiments, the cell is capable of utilizing butyric acid to produce divarinic acid. In some embodiments, the cell is capable of utilizing octanoic acid, decanoic acid, dodecanoic acid, oleic acid, palmitic acid, myristic acid or stearic acid to produce one or more olivetolic acid analogs. In some embodiments, the cell is a bacteria cell or a yeast cell. In some embodiments, the cell is a Yarrowia strain

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows biosynthesis pathways for CBG(V)A and all major cannabinoids that are derived from CBGA [THC(V)A/CBD(V)A/CBC(V)A].

FIG. 2 shows OL and OA analogs that can be synthesized using the PKSs and PKCs described herein.

FIG. 3 is a list of cannabinoids that can be synthesized using CBGA synthase(s) described herein and in combination with a CBDA, CBCA, THCA, or other synthases.

FIG. 4 is a structural model of PKC4.8 with OA bound. Amino acids in the dimer interface are shown in the monomer on the right, amino acids in the active site are shown in the left monomer (all in yellow)

FIG. 5 provides a structural model of PKS23. All PKSs described herein show very similar structural homology to PKS23. The two colors show each monomer of the dimer, and the putative binding of substrate is also shown.

FIG. 6 provides a structural model of PKC4.8 where N- and C-terminus sequences have been modified to interact and produce a dimer: “zipped protein”.

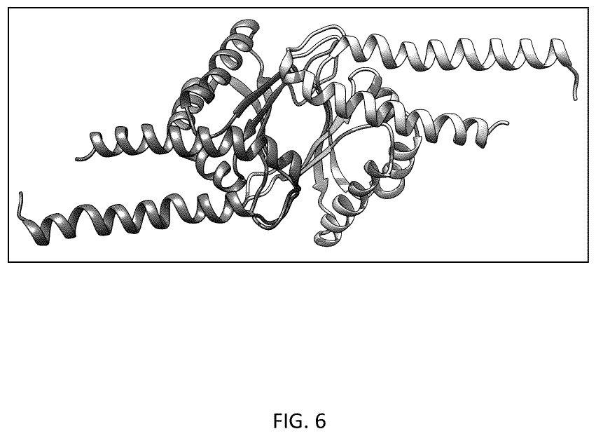

### DETAILED DESCRIPTION OF THE INVENTION

### Polyketide Synthases

Some aspects of the present disclosure are directed to a polyketide synthase (type III) comprising an amino acid sequence with at least 70% identity to SEQ ID NO: 2, 3, 4, 5, 6, 7, 8, or 68 wherein the polyketide synthase has polyketide synthase (PKS) activity. In some embodiments, the polyketide synthase comprises an amino acid sequence with at least 75%, 80%, 85%, 90%, 95%, 99%, 99.5%, or 99.9% identity to SEQ ID NO: 2, 3, 4, 5, 6, 7, 8, or 68.

Polyketide synthases catalyze the sequential condensation of acetate units to an acceptor molecule to produce a large number of natural products through the intermediacy of a polyketide. Three different classes of polyketide synthases are known, Type I, II and III (See. E.g., US20190078098; Austin, M. B. and J. P. Noel. Natural Product Reports, 2002. 20(1): p. 79-110; Lim, Y., et al. Molecules, 2016. 21(6): p. 806; Yu, D., et al. IUBMB Life, 2012. 64(4): p. 285-295). A type III polyketide synthase (PKS1) that was identified in C. sativa condenses hexanoyl-CoA with three malonyl-CoAs to produce dodecanoyl-tetraketide-CoA. Unlike other Type III polyketide synthases, PKS1 was not able to cyclize the dodecanoyl-tetraketide to olivetolic acid (OA), instead, the decarboxylated product Olivetol (OL) was formed (Taura, F, Tanaka, S, Tagichi, C, Fukumizu, T, Tanaka, H, Shoyame Y, Morimnoto, S. FEBS Lett, 2009, 583, 2061). Further structural and biochemical characterization of PKS1 confirmed the enzyme being a homodimer and suggested that OL is produced through a non-enzymatic chemical aldol condensation of the dodecanoyl-tetraketide (Kearsey, LJ, Prandi, N, Karuppiah, V, Yan, C, Leys D, Toogood, H, Takano, E, Scrutton N S FEBS J. 2020, 287(8) 1511-1524). It was later shown that in Cannabis, dodecanoyl-tetraketide-CoA is condensed to OA by the action of a separate enzyme, polyketide cyclase PKC1 (Gagne S J, Stout, J M, Liu E, Boubakir Z, Clark S M, Page J E PNAS, 2012, 109(31), 12811-12816).

Type III PKSs are able to produce a wide diversity of polyketide products by using a variety of CoA-containing precursors as a starting unit. These starters range from small aliphatic molecules, such as acetyl-CoA, to larger ring-containing compounds derived from the phenylpropanoid pathway, such as 4-coumaroyl-CoA. Often, these CoA molecules are formed through the function of acid CoA ligases (or synthase) that convert carboxylic acids into corresponding CoA thioesters (Shimizu Y, Ogata, H, Goto, S ChemBioChem 2017, 18, 50-65)

As used herein, “polyketide synthase (PKS) activity” refers to the ability of an enzyme to produce a polyketide from an acyl-CoA precursor. In some embodiments, the polyketide synthase has at least 10%, 15%, 20%, 25%, 30%, 35%, 40%, 45%, 50%, 55%, 60%, 65%, 70%, 75%, 80%, 85%, 90%, 95%, or substantially 100% of the polyketide synthase (PKS) activity of a naturally occurring PKS (e.g., PKS1 from C. sativa). In some embodiments, the polyketide synthase has at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more polyketide synthase (PKS) activity as compared to a naturally occurring PKS (e.g., PKS1 from Cannabis).

Amino acid modifications may be amino acid substitutions, amino acid deletions and/or amino acid insertions. Amino acid substitutions may be conservative amino acid substitutions or non-conservative amino acid substitutions. A conservative replacement (i.e., also referred to as a conservative mutation, a conservative substitution, or a conservative variation) is an amino acid replacement in a protein that changes a given amino acid to a different amino acid with similar biochemical properties (e.g., charge, hydrophobicity and side-chain size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

“Identity” refers to the extent to which the sequence of two or more nucleic acids or polypeptides is the same. In some embodiments, percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity is calculated using BLAST2 with default parameters as provided by the NCBI.

In some embodiments, the amino acid sequence of the polyketide synthase comprises one amino acid modification as compared to SEQ ID NO:2. In some embodiments, the amino acid sequence of the polyketide synthase comprises two amino acid modifications as compared to SEQ ID NO:2. In some embodiments, the amino acid sequence of the polyketide synthase comprises three amino acid modifications as compared to SEQ ID NO:2. In some embodiments, the amino acid sequence of the polyketide synthase comprises four amino acid modifications as compared to SEQ ID NO: 2. In some embodiments, the amino acid sequence of the polyketide synthase comprises five amino acid modifications as compared to SEQ ID NO: 2. In some embodiments, the amino acid sequence of the polyketide synthase comprises six amino acid modifications as compared to SEQ ID NO: 2. In some embodiments, the amino acid sequence of the polyketide synthase comprises seven amino acid modifications as compared to SEQ ID NO: 2. In some embodiments, the amino acid sequence of the polyketide synthase comprises eight amino acid modifications as compared to SEQ ID NO: 2. In some embodiments, the amino acid sequence of the polyketide synthase comprises nine amino acid modifications as compared to SEQ ID NO: 2. In some embodiments, the amino acid sequence of the polyketide synthase comprises 1-10 amino acid modifications as compared to SEQ ID NO: 2. In some embodiments, the amino acid sequence of the polyketide synthase comprises 10-20 amino acid modifications as compared to SEQ ID NO: 2. In some embodiments, the amino acid sequence of the polyketide synthase comprises 20-30 amino acid modifications as compared to SEQ ID NO: 2. In some embodiments, the amino acid sequence of the polyketide synthase comprises 30-40 amino acid modifications as compared to SEQ ID NO: 2.

In some embodiments, the amino acid sequence of the polyketide synthase comprises one amino acid modification as compared to SEQ ID NO: 3. In some embodiments, the amino acid sequence of the polyketide synthase comprises two amino acid modifications as compared to SEQ ID NO: 3. In some embodiments, the amino acid sequence of the polyketide synthase comprises three amino acid modifications as compared to SEQ ID NO: 3. In some embodiments, the amino acid sequence of the polyketide synthase comprises four amino acid modifications as compared to SEQ ID NO: 3. In some embodiments, the amino acid sequence of the polyketide synthase comprises five amino acid modifications as compared to SEQ ID NO: 3. In some embodiments, the amino acid sequence of the polyketide synthase comprises six amino acid modifications as compared to SEQ ID NO: 3. In some embodiments, the amino acid sequence of the polyketide synthase comprises seven amino acid modifications as compared to SEQ ID NO: 3. In some embodiments, the amino acid sequence of the polyketide synthase comprises eight amino acid modifications as compared to SEQ ID NO: 3. In some embodiments, the amino acid sequence of the polyketide synthase comprises nine amino acid modifications as compared to SEQ ID NO: 3. In some embodiments, the amino acid sequence of the polyketide synthase comprises 1-10 amino acid modifications as compared to SEQ ID NO: 3. In some embodiments, the amino acid sequence of the polyketide synthase comprises 10-20 amino acid modifications as compared to SEQ ID NO: 3. In some embodiments, the amino acid sequence of the polyketide synthase comprises 20-30 amino acid modifications as compared to SEQ ID NO: 3. In some embodiments, the amino acid sequence of the polyketide synthase comprises 30-40 amino acid modifications as compared to SEQ ID NO: 3.

In some embodiments, the amino acid sequence of the polyketide synthase comprises one amino acid modification as compared to SEQ ID NO: 4. In some embodiments, the amino acid sequence of the polyketide synthase comprises two amino acid modifications as compared to SEQ ID NO: 4. In some embodiments, the amino acid sequence of the polyketide synthase comprises three amino acid modifications as compared to SEQ ID NO: 4. In some embodiments, the amino acid sequence of the polyketide synthase comprises four amino acid modifications as compared to SEQ ID NO: 4. In some embodiments, the amino acid sequence of the polyketide synthase comprises five amino acid modifications as compared to SEQ ID NO: 4. In some embodiments, the amino acid sequence of the polyketide synthase comprises six amino acid modifications as compared to SEQ ID NO: 4. In some embodiments, the amino acid sequence of the polyketide synthase comprises seven amino acid modifications as compared to SEQ ID NO: 4. In some embodiments, the amino acid sequence of the polyketide synthase comprises eight amino acid modifications as compared to SEQ ID NO: 4. In some embodiments, the amino acid sequence of the polyketide synthase comprises nine amino acid modifications as compared to SEQ ID NO: 4. In some embodiments, the amino acid sequence of the polyketide synthase comprises 1-10 amino acid modifications as compared to SEQ ID NO: 4. In some embodiments, the amino acid sequence of the polyketide synthase comprises 10-20 amino acid modifications as compared to SEQ ID NO: 4. In some embodiments, the amino acid sequence of the polyketide synthase comprises 20-30 amino acid modifications as compared to SEQ ID NO: 4. In some embodiments, the amino acid sequence of the polyketide synthase comprises 30-40 amino acid modifications as compared to SEQ ID NO: 4.

In some embodiments, the amino acid sequence of the polyketide synthase comprises one amino acid modification as compared to SEQ ID NO: 5. In some embodiments, the amino acid sequence of the polyketide synthase comprises two amino acid modifications as compared to SEQ ID NO: 5. In some embodiments, the amino acid sequence of the polyketide synthase comprises three amino acid modifications as compared to SEQ ID NO: 5. In some embodiments, the amino acid sequence of the polyketide synthase comprises four amino acid modifications as compared to SEQ ID NO: 5. In some embodiments, the amino acid sequence of the polyketide synthase comprises five amino acid modifications as compared to SEQ ID NO: 5. In some embodiments, the amino acid sequence of the polyketide synthase comprises six amino acid modifications as compared to SEQ ID NO: 5. In some embodiments, the amino acid sequence of the polyketide synthase comprises seven amino acid modifications as compared to SEQ ID NO: 5. In some embodiments, the amino acid sequence of the polyketide synthase comprises eight amino acid modifications as compared to SEQ ID NO: 5. In some embodiments, the amino acid sequence of the polyketide synthase comprises nine amino acid modifications as compared to SEQ ID NO: 5. In some embodiments, the amino acid sequence of the polyketide synthase comprises 1-10 amino acid modifications as compared to SEQ ID NO: 5. In some embodiments, the amino acid sequence of the polyketide synthase comprises 10-20 amino acid modifications as compared to SEQ ID NO: 5. In some embodiments, the amino acid sequence of the polyketide synthase comprises 20-30 amino acid modifications as compared to SEQ ID NO: 5. In some embodiments, the amino acid sequence of the polyketide synthase comprises 30-40 amino acid modifications as compared to SEQ ID NO: 5.

In some embodiments, the amino acid sequence of the polyketide synthase comprises one amino acid modification as compared to SEQ ID NO: 6. In some embodiments, the amino acid sequence of the polyketide synthase comprises two amino acid modifications as compared to SEQ ID NO: 6. In some embodiments, the amino acid sequence of the polyketide synthase comprises three amino acid modifications as compared to SEQ ID NO: 6. In some embodiments, the amino acid sequence of the polyketide synthase comprises four amino acid modifications as compared to SEQ ID NO: 6. In some embodiments, the amino acid sequence of the polyketide synthase comprises five amino acid modifications as compared to SEQ ID NO: 6. In some embodiments, the amino acid sequence of the polyketide synthase comprises six amino acid modifications as compared to SEQ ID NO: 6. In some embodiments, the amino acid sequence of the polyketide synthase comprises seven amino acid modifications as compared to SEQ ID NO: 6. In some embodiments, the amino acid sequence of the polyketide synthase comprises eight amino acid modifications as compared to SEQ ID NO: 6. In some embodiments, the amino acid sequence of the polyketide synthase comprises nine amino acid modifications as compared to SEQ ID NO: 6. In some embodiments, the amino acid sequence of the polyketide synthase comprises 1-10 amino acid modifications as compared to SEQ ID NO: 6. In some embodiments, the amino acid sequence of the polyketide synthase comprises 10-20 amino acid modifications as compared to SEQ ID NO: 6. In some embodiments, the amino acid sequence of the polyketide synthase comprises 20-30 amino acid modifications as compared to SEQ ID NO: 6. In some embodiments, the amino acid sequence of the polyketide synthase comprises 30-40 amino acid modifications as compared to SEQ ID NO: 6.

In some embodiments, the amino acid sequence of the polyketide synthase comprises one amino acid modification as compared to SEQ ID NO: 7. In some embodiments, the amino acid sequence of the polyketide synthase comprises two amino acid modifications as compared to SEQ ID NO: 7. In some embodiments, the amino acid sequence of the polyketide synthase comprises three amino acid modifications as compared to SEQ ID NO: 7. In some embodiments, the amino acid sequence of the polyketide synthase comprises four amino acid modifications as compared to SEQ ID NO: 7. In some embodiments, the amino acid sequence of the polyketide synthase comprises five amino acid modifications as compared to SEQ ID NO: 7. In some embodiments, the amino acid sequence of the polyketide synthase comprises six amino acid modifications as compared to SEQ ID NO: 7. In some embodiments, the amino acid sequence of the polyketide synthase comprises seven amino acid modifications as compared to SEQ ID NO: 7. In some embodiments, the amino acid sequence of the polyketide synthase comprises eight amino acid modifications as compared to SEQ ID NO: 7. In some embodiments, the amino acid sequence of the polyketide synthase comprises nine amino acid modifications as compared to SEQ ID NO: 7. In some embodiments, the amino acid sequence of the polyketide synthase comprises 1-10 amino acid modifications as compared to SEQ ID NO: 7. In some embodiments, the amino acid sequence of the polyketide synthase comprises 10-20 amino acid modifications as compared to SEQ ID NO: 7. In some embodiments, the amino acid sequence of the polyketide synthase comprises 20-30 amino acid modifications as compared to SEQ ID NO: 7. In some embodiments, the amino acid sequence of the polyketide synthase comprises 30-40 amino acid modifications as compared to SEQ ID NO: 7.

In some embodiments, the amino acid sequence of the polyketide synthase comprises one amino acid modification as compared to SEQ ID NO: 8. In some embodiments, the amino acid sequence of the polyketide synthase comprises two amino acid modifications as compared to SEQ ID NO: 8. In some embodiments, the amino acid sequence of the polyketide synthase comprises three amino acid modifications as compared to SEQ ID NO: 8. In some embodiments, the amino acid sequence of the polyketide synthase comprises four amino acid modifications as compared to SEQ ID NO: 8. In some embodiments, the amino acid sequence of the polyketide synthase comprises five amino acid modifications as compared to SEQ ID NO: 8. In some embodiments, the amino acid sequence of the polyketide synthase comprises six amino acid modifications as compared to SEQ ID NO: 8. In some embodiments, the amino acid sequence of the polyketide synthase comprises seven amino acid modifications as compared to SEQ ID NO: 8. In some embodiments, the amino acid sequence of the polyketide synthase comprises eight amino acid modifications as compared to SEQ ID NO: 8. In some embodiments, the amino acid sequence of the polyketide synthase comprises nine amino acid modifications as compared to SEQ ID NO: 8. In some embodiments, the amino acid sequence of the polyketide synthase comprises 1-10 amino acid modifications as compared to SEQ ID NO: 8. In some embodiments, the amino acid sequence of the polyketide synthase comprises 10-20 amino acid modifications as compared to SEQ ID NO: 8. In some embodiments, the amino acid sequence of the polyketide synthase comprises 20-30 amino acid modifications as compared to SEQ ID NO: 8. In some embodiments, the amino acid sequence of the polyketide synthase comprises 30-40 amino acid modifications as compared to SEQ ID NO: 8.

In some embodiments, the amino acid sequence of the polyketide synthase comprises one amino acid modification as compared to SEQ ID NO: 68. In some embodiments, the amino acid sequence of the polyketide synthase comprises two amino acid modifications as compared to SEQ ID NO: 68. In some embodiments, the amino acid sequence of the polyketide synthase comprises three amino acid modifications as compared to SEQ ID NO: 68. In some embodiments, the amino acid sequence of the polyketide synthase comprises four amino acid modifications as compared to SEQ ID NO: 68. In some embodiments, the amino acid sequence of the polyketide synthase comprises five amino acid modifications as compared to SEQ ID NO: 68. In some embodiments, the amino acid sequence of the polyketide synthase comprises six amino acid modifications as compared to SEQ ID NO: 68. In some embodiments, the amino acid sequence of the polyketide synthase comprises seven amino acid modifications as compared to SEQ ID NO: 68. In some embodiments, the amino acid sequence of the polyketide synthase comprises eight amino acid modifications as compared to SEQ ID NO: 68. In some embodiments, the amino acid sequence of the polyketide synthase comprises nine amino acid modifications as compared to SEQ ID NO: 68. In some embodiments, the amino acid sequence of the polyketide synthase comprises 1-10 amino acid modifications as compared to SEQ ID NO: 68. In some embodiments, the amino acid sequence of the polyketide synthase comprises 10-20 amino acid modifications as compared to SEQ ID NO: 68. In some embodiments, the amino acid sequence of the polyketide synthase comprises 20-30 amino acid modifications as compared to SEQ ID NO: 68. In some embodiments, the amino acid sequence of the polyketide synthase comprises 30-40 amino acid modifications as compared to SEQ ID NO: 68.

In some embodiments, the polyketide synthase comprises an amino acid sequence with at least one amino acid substitution as compared to SEQ ID NO: 2, wherein the amino acid substitution is located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least two amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least three amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least four amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least five amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least six amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least seven amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least eight amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least nine amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least ten amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least eleven amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, 1263, M265, M272, Y274, H313, G315, N346, S348, F382, G383, and P384. In some embodiments, the polyketide synthase comprises an amino acid sequence with at least twelve amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least thirteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least fourteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least fifteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least sixteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least seventeen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least eighteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least nineteen amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-one amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-two amino acid substitutions as compared to SEQ ID NO: 2, wherein the amino acid substitution are located in SEQ ID NO: 2 at positions selected from A106, Y140, S141, A145, L169, G171, C172, E200, T202, 1204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-three amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-four amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-five amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with at least twenty-six amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, 1263, M265, M272, Y274, H313, G315, N346, S348, F382, G383, and P384. In some embodiments, the polyketide synthase comprises an amino acid sequence with at least twenty-seven amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384. In some embodiments, the polyketide synthase comprises an amino acid sequence with twenty-eight amino acid substitutions as compared to SEQ ID NO: 2, wherein the amino acid substitution are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384.

In some embodiments, the polyketide synthase comprises an amino acid sequence with at least one amino acid substitution as compared to SEQ ID NO: 3, wherein the amino acid substitution is located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least two amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least three amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least four amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 5 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 6 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 7 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 8 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 9 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 10 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 11 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 12 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 13 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 14 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 15 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 16 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 17 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 18 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 19 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 20 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 21 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 22 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 23 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 24 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 25 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 26 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 27 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with 28 amino acid substitutions as compared to SEQ ID NO: 3, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

In some embodiments, the polyketide synthase comprises an amino acid sequence with at least one amino acid substitution as compared to SEQ ID NO: 4, wherein the amino acid substitution is located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 2 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 3 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 4 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 5 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 6 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 7 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 8 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 9 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 10 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 11 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 12 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 13 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 14 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 15 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 16 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 17 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 18 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 19 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 20 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 21 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, 1258, M260, M267, Y269, H308, G310, N341, S343, F377, G378, and P379. In some embodiments, the polyketide synthase comprises an amino acid sequence with at least 22 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 23 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 24 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 25 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 26 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with at least 27 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379. In some embodiments, the polyketide synthase comprises an amino acid sequence with 28 amino acid substitutions as compared to SEQ ID NO: 4, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

In some embodiments, the polyketide synthase comprises an amino acid sequence with at least one amino acid substitution as compared to SEQ ID NO: 5, wherein the amino acid substitution is located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 2 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 3 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 4 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 5 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 6 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, 1264, M266, M273, Y275, H314, G316, N347, S349, F383, G384, and P385. In some embodiments, the polyketide synthase comprises an amino acid sequence with at least 7 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 8 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 9 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 10 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 11 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, 1264, M266, M273, Y275, H314, G316, N347, S349, F383, G384, and P385. In some embodiments, the polyketide synthase comprises an amino acid sequence with at least 12 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 13 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 14 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 15 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 16 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, 1264, M266, M273, Y275, H314, G316, N347, S349, F383, G384, and P385. In some embodiments, the polyketide synthase comprises an amino acid sequence with at least 17 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 18 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 19 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 20 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 21 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, 1264, M266, M273, Y275, H314, G316, N347, S349, F383, G384, and P385. In some embodiments, the polyketide synthase comprises an amino acid sequence with at least 22 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 23 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 24 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 25 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with at least 26 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, 1264, M266, M273, Y275, H314, G316, N347, S349, F383, G384, and P385. In some embodiments, the polyketide synthase comprises an amino acid sequence with at least 27 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385. In some embodiments, the polyketide synthase comprises an amino acid sequence with 28 amino acid substitutions as compared to SEQ ID NO: 5, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385.

In some embodiments, the polyketide synthase comprises an amino acid sequence with at least one amino acid substitution as compared to SEQ ID NO: 6, wherein the amino acid substitution is located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 2 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 3 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 4 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 5 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 6 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 7 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 8 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 9 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 10 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 11 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 12 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 13 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 14 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 15 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 16 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 17 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 18 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 19 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 20 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 21 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 22 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 23 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 24 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 25 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 26 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 27 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with 28 amino acid substitutions as compared to SEQ ID NO: 6, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

In some embodiments, the polyketide synthase comprises an amino acid sequence with at least one amino acid substitution as compared to SEQ ID NO: 7, wherein the amino acid substitution is located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 2 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 3 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 4 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 5 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 6 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 7 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 8 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 9 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 10 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 11 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 12 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 13 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 14 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 15 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 16 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 17 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 18 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 19 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 20 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 21 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 22 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 23 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 24 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 25 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with at least 26 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, 1258, M260, M267, Y269, H309, G311, N342, S344, F379, G380, and P381. In some embodiments, the polyketide synthase comprises an amino acid sequence with at least 27 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381. In some embodiments, the polyketide synthase comprises an amino acid sequence with 28 amino acid substitutions as compared to SEQ ID NO: 7, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

In some embodiments, the polyketide synthase comprises an amino acid sequence with at least one amino acid substitution as compared to SEQ ID NO: 8, wherein the amino acid substitution is located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 2 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 3 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 4 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 5 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 6 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, 1259, M261, M268, Y270, H309, G311, N342, S344, F376, G377, and P378. In some embodiments, the polyketide synthase comprises an amino acid sequence with at least 7 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 8 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 9 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 10 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 11 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, 1259, M261, M268, Y270, H309, G311, N342, S344, F376, G377, and P378. In some embodiments, the polyketide synthase comprises an amino acid sequence with at least 12 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 13 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 14 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 15 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 16 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, 1259, M261, M268, Y270, H309, G311, N342, S344, F376, G377, and P378. In some embodiments, the polyketide synthase comprises an amino acid sequence with at least 17 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 18 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 19 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 20 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 21 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, 1259, M261, M268, Y270, H309, G311, N342, S344, F376, G377, and P378. In some embodiments, the polyketide synthase comprises an amino acid sequence with at least 22 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 23 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 24 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 25 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with at least 26 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, 1259, M261, M268, Y270, H309, G311, N342, S344, F376, G377, and P378. In some embodiments, the polyketide synthase comprises an amino acid sequence with at least 27 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378. In some embodiments, the polyketide synthase comprises an amino acid sequence with 28 amino acid substitutions as compared to SEQ ID NO: 8, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378.

In some embodiments, the polyketide synthase comprises an amino acid sequence with at least one amino acid substitution as compared to SEQ ID NO: 68, wherein the amino acid substitution is located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 2 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 3 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 4 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 5 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 6 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 7 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 8 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 9 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 10 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 11 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 12 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 13 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 14 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 15 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 16 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 17 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 18 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 19 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 20 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 21 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 22 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 23 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 24 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 25 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 26 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with at least 27 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369. In some embodiments, the polyketide synthase comprises an amino acid sequence with 28 amino acid substitutions as compared to SEQ ID NO: 68, wherein the amino acid substitutions are located in SEQ ID NO: 68 at positions selected from S126, G156, C157, G193, G204, F208, G209, D210, I248, H297, G299, N330, S332, F367, G368, and P369.

In some embodiments, the polyketide synthase further comprises a tag or other sequence. In some embodiments, the polyketide synthase further comprises a cleavage sequence, a linker sequence, a solubility tag, a scaffolding tag, a dimerizable small peptide, or an affinity tag sequence. For example, the tag can be an affinity tag (e.g., HA, TAP, Myc, 6×his, Flag, GST), fluorescent or luminescent protein (e.g., EGFP, ECFP, EYFP, Cerulean, DsRed, mCherry), solubility-enhancing tag (e.g., Ubiquitin tag, a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006) or Varshavsky A. Methods Enzymol. 326: 578-593 (2000). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a tag is located at the N- or C-terminus, e.g., as an N- or C-terminal fusion. The polypeptide could comprise multiple tags. In some embodiments, a tag is cleavable, so that it can be removed from the polypeptide, e.g., by a protease. Exemplary proteases include, e.g., thrombin, TEV protease, Factor Xa, PreScission protease, etc. In some embodiments, a “self-cleaving” tag is used. See, e.g., PCT/US05/05763. In some embodiments a tag or other heterologous sequence is separated from the rest of the protein by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc. A scaffolding tag refers to a peptide that can interact with itself or another peptide or protein. Numerous small peptides that can form homo or hetero dimers have been described and have been used to co-localize enzymes (e.g Park, WM Int. J Mol Sci (2000) 21 3584; Anderson G P, Shriver-Lake L C, Liu, J L, Goldman E R, ACS Omega, 2018, 3, 4810-4815). Small tag peptides can interact with proteins to form tight complexes and have also been used to create multi-protein scaffolds (Vanderstraeten J, Briers, Y Biotechnol. Advances 2020, 44, 107627, Keasling J D et al Nature Biotechnol 2009, 27(8), 753).

In some embodiments, the polyketide synthase is capable of producing a tetraketide from one or more acyl-CoA substrates (e.g., acyl-CoA consisting of an acid with C2 to C22 carbons). In some embodiments, the acyl-CoA substrate is selected from acetyl-CoA, butyryl-CoA, Hexanoyl-CoA, Octanoyl-CoA, decanoyl-CoA, dodecanoyl-CoA, Myristoyl-CoA, Palmitoleyl-CoA, Linoleyl-CoA, Palmitoyl-CoA, and Oleyl-CoA. In some embodiments, the acyl-CoA substrate is Hexanoyl-CoA. In some embodiments, the acyl-CoA substrate is butyryl-CoA.

In some embodiments, the polyketide synthase is capable of producing a tetraketide from the acyl-CoA substrate at a higher rate than PKS1 from Cannabis sativa. In some embodiments, the polyketide synthase is capable of producing a tetraketide from the acyl-CoA substrate at a rate that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more of the tetraketide synthesis rate of PKS-1 from Cannabis sativa. In some embodiments, the polyketide synthase is capable of producing a tetraketide from the acyl-CoA substrate at a rate that is at least 10%, 15%, 20%, 25%, 30%, 35%, 40%, 45%, 50%, 55%, 60%, 65%, 70%, 75%, 80%, 85%, 90%, 95%, or substantially 100% of the tetraketide synthesis rate of PKS-1 from Cannabis sativa.

Some aspects of the present disclosure are directed to a cell comprising a polyketide synthase disclosed herein. In some embodiments, the cell is a transgenic cell (e.g., the polyketide synthase is coded by a heterologous sequence). In some embodiments, the cell is a yeast cell, in a bacterial cell, in an algae cell, or in a plant cell. In some embodiments, the cell is a yeast cell. In some embodiments, the yeast is an oleaginous yeast (e.g., a Yarrowia lipolytica strain). In some embodiments, the bacteria is Escherichia coli.

Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Scizichytrim, sp, Aurantiochytrium sp, Yarrowia lipolytica and the like. In some embodiments, the cell is a protease-deficient strain of Saccharomyces cerevisiae. In some embodiments, the cell is a eukaryotic cell other than a plant cell. In some embodiments, the cell is a plant cell. In some embodiments, the cell is a plant cell, where the plant cell is one that does not normally produce a cannabinoid, a cannabinoid derivative or analogue, a cannabinoid precursor, or a cannabinoid precursor derivative or analogue. In some embodiments, the cell is Saccharomyces cerevisiae.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

Some aspects of the present disclosure are directed to a polynucleotide coding for a polyketide synthase disclosed herein.

An expression vector or vectors can be constructed to include exogenous nucleotide sequences coding for the recombinant polypeptides described herein operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

In some embodiments, the cell comprises an exogenous nucleotide sequence coding for a recombinant polypeptide comprising an amino acid sequence having at least 70% identity to SEQ ID NO:1, 2, 3, 4, 5, 6, 7, 8, or 68. In some embodiments, the cell comprises an exogenous nucleotide sequence coding for a recombinant polypeptide comprising an amino acid sequence having at least 75%, 80%, 85%, 90%, 95%, 99%, 99.5%, or 99.9% identity to SEQ ID NO: 1, 2, 3, 4, 5, 6, 7, 8, or 68.

In some embodiments, the cell comprises an exogenous nucleotide sequence coding for a recombinant polypeptide comprising an amino acid sequence with 1-40 amino acid modifications as compared to SEQ ID NO: 1, 2, 3, 4, 5, 6, 7, 8, or 68 and, optionally, one to twenty amino acids deleted from the C-terminus or N-terminus.

In some embodiments, the cell comprises an exogenous nucleotide sequence coding for a recombinant polypeptide comprising an amino acid sequence identical to SEQ ID NO: 2 with one to twenty-eight amino acid substitutions and, optionally, one to twenty amino acids deleted from the C-terminus and/or N-terminus, wherein the amino acid substitutions are located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384.

In some embodiments, the cell comprises an exogenous nucleotide sequence coding for a recombinant polypeptide comprising an amino acid sequence identical to SEQ ID NO: 3 with one to twenty-eight amino acid substitutions and, optionally, one to twenty amino acids deleted from the C-terminus and/or N-terminus, wherein the amino acid substitutions are located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

In some embodiments, the cell comprises an exogenous nucleotide sequence coding for a recombinant polypeptide comprising an amino acid sequence identical to SEQ ID NO: 4 with one to twenty-eight amino acid substitutions and, optionally, one to twenty amino acids deleted from the C-terminus and/or N-terminus, wherein the amino acid substitutions are located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

In some embodiments, the cell comprises an exogenous nucleotide sequence coding for a recombinant polypeptide comprising an amino acid sequence identical to SEQ ID NO: 5 with one to twenty-eight amino acid substitutions and, optionally, one to twenty amino acids deleted from the C-terminus and/or N-terminus, wherein the amino acid substitutions are located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385.

In some embodiments, the cell comprises an exogenous nucleotide sequence coding for a recombinant polypeptide comprising an amino acid sequence identical to SEQ ID NO: 6 with one to twenty-eight amino acid substitutions and, optionally, one to twenty amino acids deleted from the C-terminus and/or N-terminus, wherein the amino acid substitutions are located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

In some embodiments, the cell comprises an exogenous nucleotide sequence coding for a recombinant polypeptide comprising an amino acid sequence identical to SEQ ID NO: 7 with one to twenty-eight amino acid substitutions and, optionally, one to twenty amino acids deleted from the C-terminus and/or N-terminus, wherein the amino acid substitutions are located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

In some embodiments, the cell comprises an exogenous nucleotide sequence coding for a recombinant polypeptide comprising an amino acid sequence identical to SEQ ID NO: 8 with one to twenty-eight amino acid substitutions and, optionally, one to twenty amino acids deleted from the C-terminus and/or N-terminus, wherein the amino acid substitutions are located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378.

In some embodiments, the cell comprises an exogenous nucleotide sequence coding for a recombinant polypeptide comprising an amino acid sequence identical to SEQ ID NO: 68 with one to twenty-eight amino acid substitutions and, optionally, one to twenty amino acids deleted from the C-terminus and/or N-terminus, wherein the amino acid substitutions are located in SEQ ID NO: 68 at positions selected from S126, G156, C157, G193, G204, F208, G209, D210, I248, H297, G299, N330, S332, F367, G368, and P369.

In some embodiments, the recombinant polypeptide further comprises a fusion domain. The fusion domain is not limited and may be any fusion domain disclosed herein. In some embodiments, the fusion domain is a domain useful for affinity chromatography. In some embodiments, the fusion domain targets the protein to a specific compartment of the cell such as the ER, vacuole, Golgi, peroxisome, lipid body (e.g., oleosome), or targets secretion of the protein from the cell into the outer membrane, periplasmic space or the culture media. In other embodiments the recombinant polypeptide or its mutants described herein is fused to an acyl-CoA synthase.

### Polyketide Cyclases

Some aspects of the present disclosure are directed to a polyketide cyclase (PKC) comprising an amino acid sequence with at least 70% identity to SEQ ID NO: 9, 10, 11, 12, 69, 70, 71, 72, 73, 74, 75, 76, 77, 78, 79, or 80, wherein the polyketide cyclase has polyketide cyclase (PKC) activity. In some embodiments, the polyketide cyclase comprises and amino acid sequence with at least 75%, 80%, 85%, 90%, 95%, 99%, 99.5%, or 99.9% identity to SEQ ID NO: 9, 10, 11, 12, 69, 70, 71, 72, 73, 74, 75, 76, 77, 78, 79, or 80.

As used herein, “PKC activity” refers to ability to cyclize a polyketide (i.e tetraketide) to an aromatic hydroxy acid (e.g., olivetolic acid or divarinic acid). In some embodiments, the PKC has at least 10%, 15%, 20%, 25%, 30%, 35%, 40%, 45%, 50%, 55%, 60%, 65%, 70%, 75%, 80%, 85%, 90%, 95%, or substantially 100% of the PKC activity of a naturally occurring PKC (e.g., PKC1 from Cannabis, PKC4 from Cannabis). In some embodiments, the PKC has at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more PKC activity as compared to a naturally occurring PKC (e.g., PKC from Cannabis).

In some embodiments, the amino acid sequence of the polyketide cyclase comprises at least one amino acid modification as compared to SEQ ID NO: 9, 10, 11, 12, 69, 70, 71, 72, 73, 74, 75, 76, 77, 78, 79, or 80. As used herein, an amino acid modification may be an insertion, deletion, or substitution.

In some embodiments, the amino acid sequence of the polyketide cyclase has at least 70% identity to SEQ ID NO: 40, 41, 42, 43, 44, or 45. In some embodiments, the amino acid sequence of the polyketide cyclase has at least 75%, 80%, 85%, 90%, 95%, 99%, 99.5%, or 99.9% identity to SEQ ID NO: 40, 41, 42, 43, 44, or 45. In some embodiments, the amino acid sequence of the polyketide cyclase comprises SEQ ID NO: 40, 41, 42, 43, 44, 45 or 46.

In some embodiments, the amino acid sequence of the polyketide cyclase comprises at least 1 amino acid modifications as compared to SEQ ID NO: 10. In some embodiments, the amino acid sequence of the polyketide cyclase comprises at least 2 amino acid modifications as compared to SEQ ID NO: 10. In some embodiments, the amino acid sequence of the polyketide cyclase comprises at least 3 amino acid modifications as compared to SEQ ID NO: 10. In some embodiments, the amino acid sequence of the polyketide cyclase comprises at least 4 amino acid modifications as compared to SEQ ID NO: 10. In some embodiments, the amino acid sequence of the polyketide cyclase comprises at least 5 amino acid modifications as compared to SEQ ID NO: 10. In some embodiments, the amino acid sequence of the polyketide cyclase comprises at least 6 amino acid modifications as compared to SEQ ID NO: 10. In some embodiments, the amino acid sequence of the polyketide cyclase comprises at least 7 amino acid modifications as compared to SEQ ID NO: 10. In some embodiments, the amino acid sequence of the polyketide cyclase comprises at least 8 amino acid modifications as compared to SEQ ID NO: 10. In some embodiments, the amino acid sequence of the polyketide cyclase comprises at least 9 amino acid modifications as compared to SEQ ID NO: 10. In some embodiments, the amino acid sequence of the polyketide cyclase comprises at least 10 amino acid modifications as compared to SEQ ID NO: 10. In some embodiments, the amino acid sequence of the polyketide cyclase comprises at least 10-20 amino acid modifications as compared to SEQ ID NO: 10. In some embodiments, the amino acid sequence of the polyketide cyclase comprises at least 20-30 amino acid modifications as compared to SEQ ID NO: 10. In some embodiments, the amino acid sequence of the polyketide cyclase comprises a 1-30, 1-20, 1-10, or 1-5 amino acid C-terminus or N-terminus truncation as compared to SEQ ID NO: 10.

In some embodiments, the amino acid sequence of the polyketide cyclase comprises at least 1 amino acid modifications as compared to SEQ ID NO: 11. In some embodiments, the amino acid sequence of the polyketide cyclase comprises at least 2 amino acid modifications as compared to SEQ ID NO: 11. In some embodiments, the amino acid sequence of the polyketide cyclase comprises at least 3 amino acid modifications as compared to SEQ ID NO: 11. In some embodiments, the amino acid sequence of the polyketide cyclase comprises at least 4 amino acid modifications as compared to SEQ ID NO: 11. In some embodiments, the amino acid sequence of the polyketide cyclase comprises at least 5 amino acid modifications as compared to SEQ ID NO: 11. In some embodiments, the amino acid sequence of the polyketide cyclase comprises at least 6 amino acid modifications as compared to SEQ ID NO: 11. In some embodiments, the amino acid sequence of the polyketide cyclase comprises at least 7 amino acid modifications as compared to SEQ ID NO: 11. In some embodiments, the amino acid sequence of the polyketide cyclase comprises at least 8 amino acid modifications as compared to SEQ ID NO: 11. In some embodiments, the amino acid sequence of the polyketide cyclase comprises at least 9 amino acid modifications as compared to SEQ ID NO: 11. In some embodiments, the amino acid sequence of the polyketide cyclase comprises at least 10 amino acid modifications as compared to SEQ ID NO: 11. In some embodiments, the amino acid sequence of the polyketide cyclase comprises at least 10-20 amino acid modifications as compared to SEQ ID NO: 11. In some embodiments, the amino acid sequence of the polyketide cyclase comprises at least 20-30 amino acid modifications as compared to SEQ ID NO: 11. In some embodiments, the amino acid sequence of the polyketide cyclase comprises a 1-30, 1-20, 1-10, or 1-5 amino acid C-terminus or N-terminus truncation as compared to SEQ ID NO: 11.

In some embodiments, the amino acid sequence of the polyketide cyclase comprises at least 1 amino acid modifications as compared to SEQ ID NO: 69. In some embodiments, the amino acid sequence of the polyketide cyclase comprises at least 2 amino acid modifications as compared to SEQ ID NO: 69. In some embodiments, the amino acid sequence of the polyketide cyclase comprises at least 3 amino acid modifications as compared to SEQ ID NO: 69. In some embodiments, the amino acid sequence of the polyketide cyclase comprises at least 4 amino acid modifications as compared to SEQ ID NO: 69. In some embodiments, the amino acid sequence of the polyketide cyclase comprises at least 5 amino acid modifications as compared to SEQ ID NO: 69. In some embodiments, the amino acid sequence of the polyketide cyclase comprises at least 6 amino acid modifications as compared to SEQ ID NO: 69. In some embodiments, the amino acid sequence of the polyketide cyclase comprises at least 7 amino acid modifications as compared to SEQ ID NO: 69. In some embodiments, the amino acid sequence of the polyketide cyclase comprises at least 8 amino acid modifications as compared to SEQ ID NO: 69. In some embodiments, the amino acid sequence of the polyketide cyclase comprises at least 9 amino acid modifications as compared to SEQ ID NO: 69. In some embodiments, the amino acid sequence of the polyketide cyclase comprises at least 10 amino acid modifications as compared to SEQ ID NO: 69. In some embodiments, the amino acid sequence of the polyketide cyclase comprises at least 10-20 amino acid modifications as compared to SEQ ID NO: 69. In some embodiments, the amino acid sequence of the polyketide cyclase comprises at least 20-30 amino acid modifications as compared to SEQ ID NO: 69. In some embodiments, the amino acid sequence of the polyketide cyclase comprises a 1-30, 1-20, 1-10, or 1-5 amino acid C-terminus or N-terminus truncation as compared to SEQ ID NO: 69.

In some embodiments, the amino acid sequence of the polyketide cyclase comprises at least 1 amino acid modifications as compared to SEQ ID NO: 70. In some embodiments, the amino acid sequence of the polyketide cyclase comprises at least 2 amino acid modifications as compared to SEQ ID NO: 70. In some embodiments, the amino acid sequence of the polyketide cyclase comprises at least 3 amino acid modifications as compared to SEQ ID NO: 70. In some embodiments, the amino acid sequence of the polyketide cyclase comprises at least 4 amino acid modifications as compared to SEQ ID NO: 70. In some embodiments, the amino acid sequence of the polyketide cyclase comprises at least 5 amino acid modifications as compared to SEQ ID NO: 70. In some embodiments, the amino acid sequence of the polyketide cyclase comprises at least 6 amino acid modifications as compared to SEQ ID NO: 70. In some embodiments, the amino acid sequence of the polyketide cyclase comprises at least 7 amino acid modifications as compared to SEQ ID NO: 70. In some embodiments, the amino acid sequence of the polyketide cyclase comprises at least 8 amino acid modifications as compared to SEQ ID NO: 70. In some embodiments, the amino acid sequence of the polyketide cyclase comprises at least 9 amino acid modifications as compared to SEQ ID NO: 70. In some embodiments, the amino acid sequence of the polyketide cyclase comprises at least 10 amino acid modifications as compared to SEQ ID NO: 70. In some embodiments, the amino acid sequence of the polyketide cyclase comprises at least 10-20 amino acid modifications as compared to SEQ ID NO: 70. In some embodiments, the amino acid sequence of the polyketide cyclase comprises at least 20-30 amino acid modifications as compared to SEQ ID NO: 70. In some embodiments, the amino acid sequence of the polyketide cyclase comprises a 1-30, 1-20, 1-10, or 1-5 amino acid C-terminus or N-terminus truncation as compared to SEQ ID NO: 70.

In some embodiments, the amino acid sequence of the polyketide cyclase comprises at least 1 amino acid modifications as compared to SEQ ID NO: 71. In some embodiments, the amino acid sequence of the polyketide cyclase comprises at least 2 amino acid modifications as compared to SEQ ID NO: 71. In some embodiments, the amino acid sequence of the polyketide cyclase comprises at least 3 amino acid modifications as compared to SEQ ID NO: 71. In some embodiments, the amino acid sequence of the polyketide cyclase comprises at least 4 amino acid modifications as compared to SEQ ID NO: 71. In some embodiments, the amino acid sequence of the polyketide cyclase comprises at least 5 amino acid modifications as compared to SEQ ID NO: 71. In some embodiments, the amino acid sequence of the polyketide cyclase comprises at least 6 amino acid modifications as compared to SEQ ID NO: 71. In some embodiments, the amino acid sequence of the polyketide cyclase comprises at least 7 amino acid modifications as compared to SEQ ID NO: 71. In some embodiments, the amino acid sequence of the polyketide cyclase comprises at least 8 amino acid modifications as compared to SEQ ID NO: 71. In some embodiments, the amino acid sequence of the polyketide cyclase comprises at least 9 amino acid modifications as compared to SEQ ID NO: 71. In some embodiments, the amino acid sequence of the polyketide cyclase comprises at least 10 amino acid modifications as compared to SEQ ID NO: 71. In some embodiments, the amino acid sequence of the polyketide cyclase comprises at least 10-20 amino acid modifications as compared to SEQ ID NO: 71. In some embodiments, the amino acid sequence of the polyketide cyclase comprises at least 20-30 amino acid modifications as compared to SEQ ID NO: 71. In some embodiments, the amino acid sequence of the polyketide cyclase comprises a 1-30, 1-20, 1-10, or 1-5 amino acid C-terminus or N-terminus truncation as compared to SEQ ID NO: 71.

In some embodiments, the amino acid sequence of the polyketide cyclase comprises at least 1 amino acid modifications as compared to SEQ ID NO: 72. In some embodiments, the amino acid sequence of the polyketide cyclase comprises at least 2 amino acid modifications as compared to SEQ ID NO: 72. In some embodiments, the amino acid sequence of the polyketide cyclase comprises at least 3 amino acid modifications as compared to SEQ ID NO: 72. In some embodiments, the amino acid sequence of the polyketide cyclase comprises at least 4 amino acid modifications as compared to SEQ ID NO: 72. In some embodiments, the amino acid sequence of the polyketide cyclase comprises at least 5 amino acid modifications as compared to SEQ ID NO: 72. In some embodiments, the amino acid sequence of the polyketide cyclase comprises at least 6 amino acid modifications as compared to SEQ ID NO: 72. In some embodiments, the amino acid sequence of the polyketide cyclase comprises at least 7 amino acid modifications as compared to SEQ ID NO: 72. In some embodiments, the amino acid sequence of the polyketide cyclase comprises at least 8 amino acid modifications as compared to SEQ ID NO: 72. In some embodiments, the amino acid sequence of the polyketide cyclase comprises at least 9 amino acid modifications as compared to SEQ ID NO: 72. In some embodiments, the amino acid sequence of the polyketide cyclase comprises at least 10 amino acid modifications as compared to SEQ ID NO: 72. In some embodiments, the amino acid sequence of the polyketide cyclase comprises at least 10-20 amino acid modifications as compared to SEQ ID NO: 72. In some embodiments, the amino acid sequence of the polyketide cyclase comprises at least 20-30 amino acid modifications as compared to SEQ ID NO: 72. In some embodiments, the amino acid sequence of the polyketide cyclase comprises a 1-30, 1-20, 1-10, or 1-5 amino acid C-terminus or N-terminus truncation as compared to SEQ ID NO: 72.

In some embodiments, the amino acid sequence of the polyketide cyclase comprises at least 1 amino acid modifications as compared to SEQ ID NO: 73. In some embodiments, the amino acid sequence of the polyketide cyclase comprises at least 2 amino acid modifications as compared to SEQ ID NO: 73. In some embodiments, the amino acid sequence of the polyketide cyclase comprises at least 3 amino acid modifications as compared to SEQ ID NO: 73. In some embodiments, the amino acid sequence of the polyketide cyclase comprises at least 4 amino acid modifications as compared to SEQ ID NO: 73. In some embodiments, the amino acid sequence of the polyketide cyclase comprises at least 5 amino acid modifications as compared to SEQ ID NO: 73. In some embodiments, the amino acid sequence of the polyketide cyclase comprises at least 6 amino acid modifications as compared to SEQ ID NO: 73. In some embodiments, the amino acid sequence of the polyketide cyclase comprises at least 7 amino acid modifications as compared to SEQ ID NO: 73. In some embodiments, the amino acid sequence of the polyketide cyclase comprises at least 8 amino acid modifications as compared to SEQ ID NO: 73. In some embodiments, the amino acid sequence of the polyketide cyclase comprises at least 9 amino acid modifications as compared to SEQ ID NO: 73. In some embodiments, the amino acid sequence of the polyketide cyclase comprises at least 10 amino acid modifications as compared to SEQ ID NO: 73. In some embodiments, the amino acid sequence of the polyketide cyclase comprises at least 10-20 amino acid modifications as compared to SEQ ID NO: 73. In some embodiments, the amino acid sequence of the polyketide cyclase comprises at least 20-30 amino acid modifications as compared to SEQ ID NO: 73. In some embodiments, the amino acid sequence of the polyketide cyclase comprises a 1-30, 1-20, 1-10, or 1-5 amino acid C-terminus or N-terminus truncation as compared to SEQ ID NO: 73.

In some embodiments, the amino acid sequence of the polyketide cyclase comprises at least 1 amino acid modifications as compared to SEQ ID NO: 74. In some embodiments, the amino acid sequence of the polyketide cyclase comprises at least 2 amino acid modifications as compared to SEQ ID NO: 74. In some embodiments, the amino acid sequence of the polyketide cyclase comprises at least 3 amino acid modifications as compared to SEQ ID NO: 74. In some embodiments, the amino acid sequence of the polyketide cyclase comprises at least 4 amino acid modifications as compared to SEQ ID NO: 74. In some embodiments, the amino acid sequence of the polyketide cyclase comprises at least 5 amino acid modifications as compared to SEQ ID NO: 74. In some embodiments, the amino acid sequence of the polyketide cyclase comprises at least 6 amino acid modifications as compared to SEQ ID NO: 74. In some embodiments, the amino acid sequence of the polyketide cyclase comprises at least 7 amino acid modifications as compared to SEQ ID NO: 74. In some embodiments, the amino acid sequence of the polyketide cyclase comprises at least 8 amino acid modifications as compared to SEQ ID NO: 74. In some embodiments, the amino acid sequence of the polyketide cyclase comprises at least 9 amino acid modifications as compared to SEQ ID NO: 74. In some embodiments, the amino acid sequence of the polyketide cyclase comprises at least 10 amino acid modifications as compared to SEQ ID NO: 74. In some embodiments, the amino acid sequence of the polyketide cyclase comprises at least 10-20 amino acid modifications as compared to SEQ ID NO: 74. In some embodiments, the amino acid sequence of the polyketide cyclase comprises at least 20-30 amino acid modifications as compared to SEQ ID NO: 74. In some embodiments, the amino acid sequence of the polyketide cyclase comprises a 1-30, 1-20, 1-10, or 1-5 amino acid C-terminus or N-terminus truncation as compared to SEQ ID NO: 74.

In some embodiments, the amino acid sequence of the polyketide cyclase comprises at least 1 amino acid modifications as compared to SEQ ID NO: 75. In some embodiments, the amino acid sequence of the polyketide cyclase comprises at least 2 amino acid modifications as compared to SEQ ID NO: 75. In some embodiments, the amino acid sequence of the polyketide cyclase comprises at least 3 amino acid modifications as compared to SEQ ID NO: 75. In some embodiments, the amino acid sequence of the polyketide cyclase comprises at least 4 amino acid modifications as compared to SEQ ID NO: 75. In some embodiments, the amino acid sequence of the polyketide cyclase comprises at least 5 amino acid modifications as compared to SEQ ID NO: 75. In some embodiments, the amino acid sequence of the polyketide cyclase comprises at least 6 amino acid modifications as compared to SEQ ID NO: 75. In some embodiments, the amino acid sequence of the polyketide cyclase comprises at least 7 amino acid modifications as compared to SEQ ID NO: 75. In some embodiments, the amino acid sequence of the polyketide cyclase comprises at least 8 amino acid modifications as compared to SEQ ID NO: 75. In some embodiments, the amino acid sequence of the polyketide cyclase comprises at least 9 amino acid modifications as compared to SEQ ID NO: 75. In some embodiments, the amino acid sequence of the polyketide cyclase comprises at least 10 amino acid modifications as compared to SEQ ID NO: 75. In some embodiments, the amino acid sequence of the polyketide cyclase comprises at least 10-20 amino acid modifications as compared to SEQ ID NO: 75. In some embodiments, the amino acid sequence of the polyketide cyclase comprises at least 20-30 amino acid modifications as compared to SEQ ID NO: 75. In some embodiments, the amino acid sequence of the polyketide cyclase comprises a 1-30, 1-20, 1-10, or 1-5 amino acid C-terminus or N-terminus truncation as compared to SEQ ID NO: 75.

In some embodiments, the amino acid sequence of the polyketide cyclase comprises at least 1 amino acid modifications as compared to SEQ ID NO: 76. In some embodiments, the amino acid sequence of the polyketide cyclase comprises at least 2 amino acid modifications as compared to SEQ ID NO: 76. In some embodiments, the amino acid sequence of the polyketide cyclase comprises at least 3 amino acid modifications as compared to SEQ ID NO: 76. In some embodiments, the amino acid sequence of the polyketide cyclase comprises at least 4 amino acid modifications as compared to SEQ ID NO: 76. In some embodiments, the amino acid sequence of the polyketide cyclase comprises at least 5 amino acid modifications as compared to SEQ ID NO: 76. In some embodiments, the amino acid sequence of the polyketide cyclase comprises at least 6 amino acid modifications as compared to SEQ ID NO: 76. In some embodiments, the amino acid sequence of the polyketide cyclase comprises at least 7 amino acid modifications as compared to SEQ ID NO: 76. In some embodiments, the amino acid sequence of the polyketide cyclase comprises at least 8 amino acid modifications as compared to SEQ ID NO: 76. In some embodiments, the amino acid sequence of the polyketide cyclase comprises at least 9 amino acid modifications as compared to SEQ ID NO: 76. In some embodiments, the amino acid sequence of the polyketide cyclase comprises at least 10 amino acid modifications as compared to SEQ ID NO: 76. In some embodiments, the amino acid sequence of the polyketide cyclase comprises at least 10-20 amino acid modifications as compared to SEQ ID NO: 76. In some embodiments, the amino acid sequence of the polyketide cyclase comprises at least 20-30 amino acid modifications as compared to SEQ ID NO: 76. In some embodiments, the amino acid sequence of the polyketide cyclase comprises a 1-30, 1-20, 1-10, or 1-5 amino acid C-terminus or N-terminus truncation as compared to SEQ ID NO: 76.

In some embodiments, the amino acid sequence of the polyketide cyclase comprises at least 1 amino acid modifications as compared to SEQ ID NO: 77. In some embodiments, the amino acid sequence of the polyketide cyclase comprises at least 2 amino acid modifications as compared to SEQ ID NO: 77. In some embodiments, the amino acid sequence of the polyketide cyclase comprises at least 3 amino acid modifications as compared to SEQ ID NO: 77. In some embodiments, the amino acid sequence of the polyketide cyclase comprises at least 4 amino acid modifications as compared to SEQ ID NO: 77. In some embodiments, the amino acid sequence of the polyketide cyclase comprises at least 5 amino acid modifications as compared to SEQ ID NO: 77. In some embodiments, the amino acid sequence of the polyketide cyclase comprises at least 6 amino acid modifications as compared to SEQ ID NO: 77. In some embodiments, the amino acid sequence of the polyketide cyclase comprises at least 7 amino acid modifications as compared to SEQ ID NO: 77. In some embodiments, the amino acid sequence of the polyketide cyclase comprises at least 8 amino acid modifications as compared to SEQ ID NO: 77. In some embodiments, the amino acid sequence of the polyketide cyclase comprises at least 9 amino acid modifications as compared to SEQ ID NO: 77. In some embodiments, the amino acid sequence of the polyketide cyclase comprises at least 10 amino acid modifications as compared to SEQ ID NO: 77. In some embodiments, the amino acid sequence of the polyketide cyclase comprises at least 10-20 amino acid modifications as compared to SEQ ID NO: 77. In some embodiments, the amino acid sequence of the polyketide cyclase comprises at least 20-30 amino acid modifications as compared to SEQ ID NO: 77. In some embodiments, the amino acid sequence of the polyketide cyclase comprises a 1-30, 1-20, 1-10, or 1-5 amino acid C-terminus or N-terminus truncation as compared to SEQ ID NO: 77.

In some embodiments, the amino acid sequence of the polyketide cyclase comprises at least 1 amino acid modifications as compared to SEQ ID NO: 78. In some embodiments, the amino acid sequence of the polyketide cyclase comprises at least 2 amino acid modifications as compared to SEQ ID NO: 78. In some embodiments, the amino acid sequence of the polyketide cyclase comprises at least 3 amino acid modifications as compared to SEQ ID NO: 78. In some embodiments, the amino acid sequence of the polyketide cyclase comprises at least 4 amino acid modifications as compared to SEQ ID NO: 78. In some embodiments, the amino acid sequence of the polyketide cyclase comprises at least 5 amino acid modifications as compared to SEQ ID NO: 78. In some embodiments, the amino acid sequence of the polyketide cyclase comprises at least 6 amino acid modifications as compared to SEQ ID NO: 78. In some embodiments, the amino acid sequence of the polyketide cyclase comprises at least 7 amino acid modifications as compared to SEQ ID NO: 78. In some embodiments, the amino acid sequence of the polyketide cyclase comprises at least 8 amino acid modifications as compared to SEQ ID NO: 78. In some embodiments, the amino acid sequence of the polyketide cyclase comprises at least 9 amino acid modifications as compared to SEQ ID NO: 78. In some embodiments, the amino acid sequence of the polyketide cyclase comprises at least 10 amino acid modifications as compared to SEQ ID NO: 78. In some embodiments, the amino acid sequence of the polyketide cyclase comprises at least 10-20 amino acid modifications as compared to SEQ ID NO: 78. In some embodiments, the amino acid sequence of the polyketide cyclase comprises at least 20-30 amino acid modifications as compared to SEQ ID NO: 78. In some embodiments, the amino acid sequence of the polyketide cyclase comprises a 1-30, 1-20, 1-10, or 1-5 amino acid C-terminus or N-terminus truncation as compared to SEQ ID NO: 78.

In some embodiments, the amino acid sequence of the polyketide cyclase comprises at least 1 amino acid modifications as compared to SEQ ID NO: 79. In some embodiments, the amino acid sequence of the polyketide cyclase comprises at least 2 amino acid modifications as compared to SEQ ID NO: 79. In some embodiments, the amino acid sequence of the polyketide cyclase comprises at least 3 amino acid modifications as compared to SEQ ID NO: 79. In some embodiments, the amino acid sequence of the polyketide cyclase comprises at least 4 amino acid modifications as compared to SEQ ID NO: 79. In some embodiments, the amino acid sequence of the polyketide cyclase comprises at least 5 amino acid modifications as compared to SEQ ID NO: 79. In some embodiments, the amino acid sequence of the polyketide cyclase comprises at least 6 amino acid modifications as compared to SEQ ID NO: 79. In some embodiments, the amino acid sequence of the polyketide cyclase comprises at least 7 amino acid modifications as compared to SEQ ID NO: 79. In some embodiments, the amino acid sequence of the polyketide cyclase comprises at least 8 amino acid modifications as compared to SEQ ID NO: 79. In some embodiments, the amino acid sequence of the polyketide cyclase comprises at least 9 amino acid modifications as compared to SEQ ID NO: 79. In some embodiments, the amino acid sequence of the polyketide cyclase comprises at least 10 amino acid modifications as compared to SEQ ID NO: 79. In some embodiments, the amino acid sequence of the polyketide cyclase comprises at least 10-20 amino acid modifications as compared to SEQ ID NO: 79. In some embodiments, the amino acid sequence of the polyketide cyclase comprises at least 20-30 amino acid modifications as compared to SEQ ID NO: 79. In some embodiments, the amino acid sequence of the polyketide cyclase comprises a 1-30, 1-20, 1-10, or 1-5 amino acid C-terminus or N-terminus truncation as compared to SEQ ID NO: 79.

In some embodiments, the amino acid sequence of the polyketide cyclase comprises at least 1 amino acid modifications as compared to SEQ ID NO: 80. In some embodiments, the amino acid sequence of the polyketide cyclase comprises at least 2 amino acid modifications as compared to SEQ ID NO: 80. In some embodiments, the amino acid sequence of the polyketide cyclase comprises at least 3 amino acid modifications as compared to SEQ ID NO: 80. In some embodiments, the amino acid sequence of the polyketide cyclase comprises at least 4 amino acid modifications as compared to SEQ ID NO: 80. In some embodiments, the amino acid sequence of the polyketide cyclase comprises at least 5 amino acid modifications as compared to SEQ ID NO: 80. In some embodiments, the amino acid sequence of the polyketide cyclase comprises at least 6 amino acid modifications as compared to SEQ ID NO: 80. In some embodiments, the amino acid sequence of the polyketide cyclase comprises at least 7 amino acid modifications as compared to SEQ ID NO: 80. In some embodiments, the amino acid sequence of the polyketide cyclase comprises at least 8 amino acid modifications as compared to SEQ ID NO: 80. In some embodiments, the amino acid sequence of the polyketide cyclase comprises at least 9 amino acid modifications as compared to SEQ ID NO: 80. In some embodiments, the amino acid sequence of the polyketide cyclase comprises at least 10 amino acid modifications as compared to SEQ ID NO: 80. In some embodiments, the amino acid sequence of the polyketide cyclase comprises at least 10-20 amino acid modifications as compared to SEQ ID NO: 80. In some embodiments, the amino acid sequence of the polyketide cyclase comprises at least 20-30 amino acid modifications as compared to SEQ ID NO: 80. In some embodiments, the amino acid sequence of the polyketide cyclase comprises a 1-30, 1-20, 1-10, or 1-5 amino acid C-terminus or N-terminus truncation as compared to SEQ ID NO: 80.

### Discussion of Stability Mutants—Example

In some embodiments, the polyketide cyclase comprises a chimeric amino acid sequence comprising portions having at least 70% sequence identity to portions of 2 or more sequences selected from SEQ ID NO: 9, 10, 11, 12, 69, 70, 71, 72, 73, 74, 75, 76, 77, 78, 79, or 80 (e.g., a portion with 70% identity to a portion of SEQ ID NO: 9 and another portion having at least 70% identity to a portion of SEQ ID NO: 10). In some embodiments, the PKC comprises a chimeric amino acid sequence comprising portions having at least 75%, 80%, 85%, 90%, 95%, 99%, 99.5%, or 99.9% sequence identity to portions of 2 or more sequences selected from SEQ ID NO: 9, 10, 11, 12, 69, 70, 71, 72, 73, 74, 75, 76, 77, 78, 79, or 80 (e.g., a portion with 70% identity to a portion of SEQ ID NO: 9 and another portion having at least 70% identity to a portion of SEQ ID NO: 10). As used herein, a portion of a sequence can be at least 5, 10, 15, 20, 25, 30, 35, 40, 45, 50, or more contiguous amino acids.

In some embodiments, the polyketide cyclase comprises an amino acid sequence with at least one amino acid modification as compared to SEQ ID NO: 10, wherein the amino acid modification is located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 2 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 3 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 4 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 5 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 6 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 7 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 8 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 9 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 10 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, F102, D103 and K105. In some embodiments, the polyketide cyclase comprises an amino acid sequence with at least 11 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, 176, Y79, I80, L86, L88, R89, Y92, F93, L96, F99, L100, V101, F102, D103 and K105. In some embodiments, the polyketide cyclase comprises an amino acid sequence with at least 12 amino acid modifications as compared to SEQ ID NO: 10, wherein the amino acid modifications are located in SEQ ID NO: 10 at positions selected from V9, H11 V12, F13, 114, L15, M17, M29, N30, Y33, A45, Q47, L51, E52, N54-Y62, H64, I65, F66, E67, S68, 169, F70, M73, I76, Y79, I80, L86, L88, R89, Y92, F93, L96, F99, L100, V101, and F102, D103 and K105. In some embodiments, the polyketide cyclase comprises an amino acid sequence with at least 13 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 14 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 15 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 16 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, 176, Y79, I80, L86, L88, R89, Y92, F93, L96, F99, L100, V101, and F102, D103 and K105. In some embodiments, the polyketide cyclase comprises an amino acid sequence with at least 17 amino acid modifications as compared to SEQ ID NO: 10, wherein the amino acid modifications are located in SEQ ID NO: 10 at positions selected from V9, H11 V12, F13, 114, L15, M17, M29, N30, Y33, A45, Q47, L51, E52, N54-Y62, H64, I65, F66, E67, S68, 169, F70, M73, I76, Y79, I80, L86, L88, R89, Y92, F93, L96, F99, L100, V101, and F102, D103 and K105. In some embodiments, the polyketide cyclase comprises an amino acid sequence with at least 18 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 19 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 20 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 21 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 22 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, 176, Y79, I80, L86, L88, R89, Y92, F93, L96, F99, L100, V101, and F102, D103 and K105. In some embodiments, the polyketide cyclase comprises an amino acid sequence with at least 23 amino acid modifications as compared to SEQ ID NO: 10, wherein the amino acid modifications are located in SEQ ID NO: 10 at positions selected from V9, H11 V12, F13, 114, L15, M17, M29, N30, Y33, A45, Q47, L51, E52, N54-Y62, H64, I65, F66, E67, S68, 169, F70, M73, I76, Y79, I80, L86, L88, R89, Y92, F93, L96, F99, L100, V101, and F102, D103 and K105. In some embodiments, the polyketide cyclase comprises an amino acid sequence with at least 24 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 25 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 26 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 27 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, 176, Y79, I80, L86, L88, R89, Y92, F93, L96, F99, L100, V101, and F102, D103 and K105. In some embodiments, the polyketide cyclase comprises an amino acid sequence with at least 28 amino acid modifications as compared to SEQ ID NO: 10, wherein the amino acid modifications are located in SEQ ID NO: 10 at positions selected from V9, H11 V12, F13, 114, L15, M17, M29, N30, Y33, A45, Q47, L51, E52, N54-Y62, H64, I65, F66, E67, S68, 169, F70, M73, I76, Y79, I80, L86, L88, R89, Y92, F93, L96, F99, L100, V101, and F102, D103 and K105. In some embodiments, the polyketide cyclase comprises an amino acid sequence with at least 29 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 30 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 31 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105. In some embodiments, the polyketide cyclase comprises an amino acid sequence with at least 32 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, 176, Y79, I80, L86, L88, R89, Y92, F93, L96, F99, L100, V101, and F102, D103 and K105. In some embodiments, the polyketide cyclase comprises an amino acid sequence with at least 33 amino acid modifications as compared to SEQ ID NO: 10, wherein the amino acid modifications are located in SEQ ID NO: 10 at positions selected from V9, H11 V12, F13, 114, L15, M17, M29, N30, Y33, A45, Q47, L51, E52, N54-Y62, H64, I65, F66, E67, S68, 169, F70, M73, I76, Y79, I80, L86, L88, R89, Y92, F93, L96, F99, L100, V101, and F102, D103 and K105. In some embodiments, the polyketide cyclase comprises an amino acid sequence with 34 amino acid modifications as compared to SEQ ID NO: 10, wherein the amino acid modifications are located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105.

In some embodiments, the polyketide cyclase comprises an amino acid sequence with at least one amino acid modification as compared to SEQ ID NO: 11, wherein the amino acid modification is located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, F102, D103, and T105. In some embodiments, the polyketide cyclase comprises an amino acid sequence with at least 2 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 3 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, 165, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 4 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 5 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 6 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 7 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 8 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 9 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 10 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 11 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, 169, F70, V73, I76, Y79, I80, V86, F88, G89, Y92, R93, W96, L99, L100, I101, and F102, D103, and T105. In some embodiments, the polyketide cyclase comprises an amino acid sequence with at least 12 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 13 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, 165, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 14 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 15 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 16 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 17 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, 169, F70, V73, I76, Y79, I80, V86, F88, G89, Y92, R93, W96, L99, L100, I101, and F102, D103, and T105. In some embodiments, the polyketide cyclase comprises an amino acid sequence with at least 18 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 19 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, 165, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 20 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 21 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 22 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 23 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, 169, F70, V73, I76, Y79, I80, V86, F88, G89, Y92, R93, W96, L99, L100, I101, and F102, D103, and T105. In some embodiments, the polyketide cyclase comprises an amino acid sequence with at least 24 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 25 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, 165, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 26 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 27 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 28 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 29 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, 169, F70, V73, I76, Y79, I80, V86, F88, G89, Y92, R93, W96, L99, L100, I101, and F102, D103, and T105. In some embodiments, the polyketide cyclase comprises an amino acid sequence with at least 30 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 31 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, 165, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 32 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 33 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105. In some embodiments, the polyketide cyclase comprises an amino acid sequence with at least 34 amino acid modifications as compared to SEQ ID NO: 11, wherein the amino acid modifications are located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, and F102, D103, and T105.

In some embodiments, the polyketide cyclase comprises an amino acid sequence with at least one amino acid modification as compared to SEQ ID NO: 69, wherein the amino acid modification is located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 2 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 3 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 4 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 5 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 6 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 7 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 8 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 9 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 10 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 11 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 12 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 13 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 14 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 15 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 16 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 17 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 18 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 19 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 20 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 21 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 22 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 23 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 24 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 25 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 26 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97. In some embodiments, the polyketide cyclase comprises an amino acid sequence with at least 27 amino acid modifications as compared to SEQ ID NO: 69, wherein the amino acid modifications are located in SEQ ID NO: 69 at positions selected from V3, H5, L9, Y27, L45, E46, N48-Y56, H58, I59, E61, T63, F64, I70, Y73, I74, Y86, L94, F96, and D97.

In some embodiments, the polyketide cyclase comprises an amino acid sequence with at least one amino acid modification as compared to SEQ ID NO: 71, wherein the amino acid modification is located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 2 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 3 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 4 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 5 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 6 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 7 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 8 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 9 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 10 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 11 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 12 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 13 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 14 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 15 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 16 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 17 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 18 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 19 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 20 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 21 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 22 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 23 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 24 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 25 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 26 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 27 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103. In some embodiments, the polyketide cyclase comprises an amino acid sequence with at least 28 amino acid modifications as compared to SEQ ID NO: 71, wherein the amino acid modifications are located in SEQ ID NO: 71 at positions selected from V9, H11, L15, Y33, L51, E52, N54-Y62, H64, I65, E67-F70, I76, Y79, I80, Y92, L100, F102 and D103.

In some embodiments, the polyketide cyclase comprises an amino acid sequence with at least one amino acid modification as compared to SEQ ID NO: 72, wherein the amino acid modification is located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 2 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 3 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 4 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 5 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 6 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 7 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 8 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 9 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 10 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 11 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 12 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 13 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 14 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 15 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 16 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 17 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 18 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 19 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 20 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 21 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 22 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 23 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 24 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 25 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 26 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 27 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 28 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 29 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 30 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 31 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97. In some embodiments, the polyketide cyclase comprises an amino acid sequence with at least 32 amino acid modifications as compared to SEQ ID NO: 72, wherein the amino acid modifications are located in SEQ ID NO: 72 at positions selected from V3, H5-L9, Y27, A39, Q41, L45, E46, N48-Y56, H58, I59, E61, S62, F64, I70, Y73, I74, Y86, L94, F96, and D97.

In some embodiments, the polyketide cyclase comprises an amino acid sequence with at least one amino acid modification as compared to SEQ ID NO: 76, wherein the amino acid modification is located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 2 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 3 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 4 amino acid modifications as compared to SEQ ID NO: 76, wherein the amino acid modifications are located in SEQ ID NO: 76 at positions selected from V9, H11 V12, 114, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 5 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 6 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 7 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 8 amino acid modifications as compared to SEQ ID NO: 76, wherein the amino acid modifications are located in SEQ ID NO: 76 at positions selected from V9, H11 V12, 114, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 9 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 10 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 11 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 12 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 13 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 14 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 15 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 16 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 17 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 18 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 19 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 20 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 21 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 22 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 23 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 24 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 25 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 26 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 27 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 28 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 29 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 30 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 31 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103. In some embodiments, the polyketide cyclase comprises an amino acid sequence with at least 32 amino acid modifications as compared to SEQ ID NO: 76, wherein the amino acid modifications are located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103.

In some embodiments, the PKC further comprises a tag or other sequence. In some embodiments, the PKC further comprises a cleavage sequence, a linker sequence, a solubility tag, a scaffolding tag, a dimerizable small peptide, and/or an affinity tag sequence. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility or expression-enhancing tag (e.g., Ubiquitin, SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006) or Varshavsky A. Methods Enzymol. 326: 578-593 (2000). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a tag is located at the N- or C-terminus, e.g., as an N- or C-terminal fusion. The polypeptide could comprise multiple tags. In some embodiments, a tag is cleavable, so that it can be removed from the polypeptide, e.g., by a protease. Exemplary proteases include, e.g., thrombin, TEV protease, Factor Xa, PreScission protease, etc. In some embodiments, a “self-cleaving” tag is used. See, e.g., PCT/US05/05763. In some embodiments a tag or other heterologous sequence is separated from the rest of the protein by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc. A scaffolding tag refers to a peptide that can interact with itself or another peptide or protein. Numerous small peptides that can form homo or hetero dimers have been described and have been used to co-localize enzymes (e.g Park, WM Int. J Mol Sci (2000) 21 3584; Anderson G P, Shriver-Lake L C, Liu, J L, Goldman E R, ACS Omega, 2018, 3, 4810-4815). Similarly, scaffolding tag peptides can interact with proteins to form tight complexes and have also been used to create multi-protein scaffolds (Vanderstraeten J, Briers, Y Biotechnol. Advances 2020, 44, 107627, Keasling J D et al Nature Biotechnol 2009, 27(8), 753).

In some embodiments, a PKC described herein (e.g., PKC1, PKC1.1, PKC4, PKC4.8 and functional fragments, variants, and derivatives thereof) comprises a C-terminal and N-terminal small peptide that can facilitate dimerization of the enzyme (i.e., dimerizable small peptide). This modification can increase stability of the PKC by zipping the N- and C-terminus of the protein. In some embodiments, the PKC comprising a C-terminal and N-Terminal small peptide that can dimerize is selected from SEQ ID NO: 40, 41, or 42. In some embodiments, the PKC comprising a C-terminal and N-terminal small peptide that can dimerize has at least about 70%, 75%, 80%, 85%, 90%, 95%, or 99% identity to SEQ ID NO: 40, 41, or 42. In some embodiments, a PKC described herein is expressed with an N-terminal small peptide of SEQ ID NO: 47 and a C-terminal small peptide selected from SEQ ID NO: 48, 49 or 68. In some embodiments, a PKC having a C-terminal and N-terminal small peptide that can dimerize is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more stable than a PKC having the same amino acid sequence except for the C-terminal and N-terminal small peptide. In some embodiments, a PKC having a C-terminal and N-terminal small peptide that can dimerize is present in a cell at a level that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold higher than the level of a PKC having the same amino acid sequence except for the C-terminal and N-terminal small peptide.

In some embodiments, a PKC described herein (e.g., PKC1, PKC1.1, PKC4, PKC4.8 and functional fragments, variants, and derivatives thereof) comprises a ubiquitin at the N-terminal that increases solubility and expression of the PKC. In some embodiments, the PKC having ubiquitin at the N-terminal comprises the amino acid sequence of SEQ ID NO: 43, 58, or 59. In some embodiments, the PKC having ubiquitin at the N-terminal has at least about 70%, 75%, 80%, 85%, 90%, 95%, or 99% identity to SEQ ID NO: 43, 58, or 59. In some embodiments, a PKC having ubiquitin at the N-terminal is present in a cell at a level that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold higher than the level of a PKC having the same amino acid sequence but not having ubiquitin at the N-terminal. In some embodiments, a PKC having ubiquitin at the N-terminal is expressed in a cell at a level that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold higher than the level of a PKC having the same amino acid sequence but not having ubiquitin at the N-terminal. In some embodiments, a PKC having ubiquitin at the N-terminal is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold more soluble than a PKC having the same amino acid sequence but not having ubiquitin at the N-terminal.

In some embodiments, a PKC described herein (e.g., PKC1, PKC1.1, PKC4, PKC4.8 and functional fragments, variants, and derivatives thereof) comprises a C-terminal and/or an N-terminal scaffolding tag. In some embodiments, the scaffolding tag is capable of forming homodimers. In some embodiments, the scaffolding tag is capable of forming heterodimers. In some embodiments, the scaffolding tag is capable of forming both homodimers and heterodimers. In some embodiments, the scaffolding tag has the amino acid sequence of SEQ ID NO: 66 (P3) or 67 (P4). In some embodiments, the PKC having a scaffolding tag comprises the amino acid sequence of SEQ ID NO: 44 or 45. In some embodiments, the PKC having a scaffolding tag comprises an amino acid sequence having at least about 70%, 75%, 80%, 85%, 90%, 95%, or 99% identity to SEQ ID NO: 44 or 45. In some embodiments, a PKC having a scaffolding tag is expressed in a cell at a level that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold higher than the level of a PKC having the same amino acid sequence but not having a scaffolding tag. In some embodiments, a PKC having a scaffolding tag is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold more soluble than a PKC having the same amino acid sequence but not having a scaffolding tag. In some embodiments, a PKC having a scaffolding tag is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold more active than a PKC having the same amino acid sequence but not having a scaffolding tag.

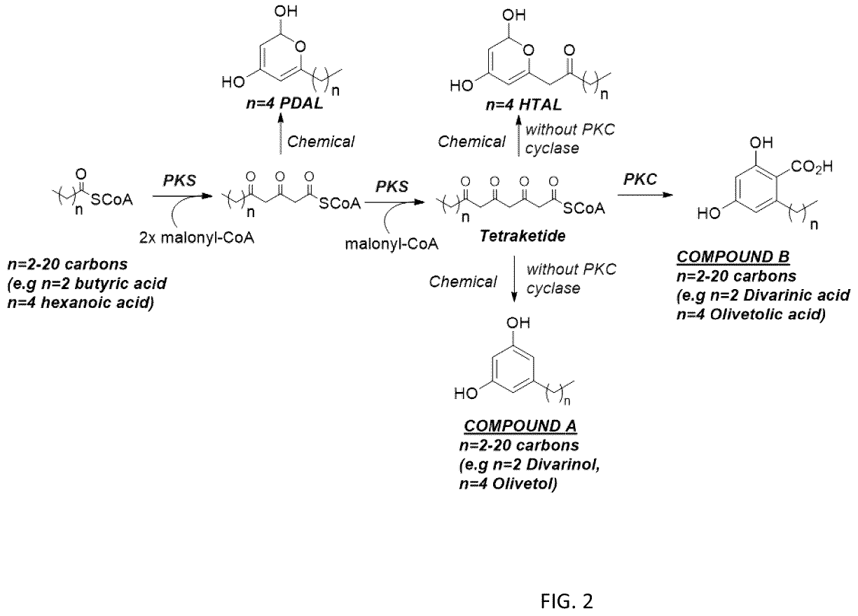

In some embodiments, the polyketide cyclase is capable of producing 6-alkyl-2,4-dihydroxy benzoic acid from a tetraketide as shown in FIG. 2 (Compound B). In other embodiments the polyketide cyclase produces olivetolic acid (OA), an OA analog, divarinic acid (DVA), or a DVA analog from a tetraketide. In some embodiments, the polyketide cyclase is capable of producing 6-alkyl-2,4-dihydroxy benzoic acid shown in compound B in FIG. 2. In some embodiments, the PKC is capable of producing OA, an OA analog, DVA, or a DVA analog at a higher rate than PKC4 (SEQ ID NO: 10). In some embodiments, the PKC is capable of producing OA, OA analog, DVA, or a DVA analog at a rate that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or higher than the rate of PKC4. In some embodiments, the PKC is capable of producing OA, an OA analog, DVA, or a DVA analog at a rate that is at least 10%, 15%, 20%, 25%, 30%, 35%, 40%, 45%, 50%, 55%, 60%, 65%, 70%, 75%, 80%, 85%, 90%, 95%, or substantially 100% of the rate of PKC4.

In some embodiments, the PKC is capable of producing a 6-alkyl-2,4-dihydroxy benzoic acid, and specifically OA, an OA analog, DVA, or a DVA analog at a higher rate than PKC4.8 (SEQ ID NO: 11). In some embodiments, the PKC is capable of producing OA, OA analog, DVA, or a DVA analog at a rate that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or higher than the rate of PKC4.8. In some embodiments, the PKC is capable of producing OA, OA analog, DVA, or a DVA analog at a rate that is at least 10%, 15%, 20%, 25%, 30%, 35%, 40%, 45%, 50%, 55%, 60%, 65%, 70%, 75%, 80%, 85%, 90%, 95%, or substantially 100% of the rate of PKC4.8.

Some aspects of the present disclosure are directed to a cell comprising a PKC disclosed herein. In some embodiments, the cell is a transgenic cell (e.g., the polyketide cyclase is coded by a heterologous sequence). In some embodiments, the cell is a yeast cell, in a bacterial cell, in an algae cell, or in a plant cell. In some embodiments, the cell is a yeast cell. In some embodiments, the yeast is an oleaginous yeast (e.g., a Yarrowia lipolytica strain). The cell is not limited and may be any suitable cell disclosed herein.

Some aspects of the present disclosure are directed to a polynucleotide coding for a PKC disclosed herein.

An expression vector or vectors can be constructed to include exogenous nucleotide sequences coding for the recombinant polypeptides described herein operably linked to expression control sequences functional in the cell. Any suitable expression vector disclosed herein may be used.

In some embodiments, the cell comprises an exogenous nucleotide sequence coding for a recombinant polypeptide comprising an amino acid sequence having at least 70% identity to SEQ ID NO: 9, 10, 11 or 12. In some embodiments, the cell comprises an exogenous nucleotide sequence coding for a recombinant polypeptide comprising an amino acid sequence having at least 75%, 80%, 85%, 90%, 95%, 99%, 99.5%, or 99.9% identity to SEQ ID NO: 9, 10, 11 or 12.

In some embodiments, the cell comprises an exogenous nucleotide sequence coding for a recombinant polypeptide comprising an amino acid sequence with 1-40 amino acid modifications as compared to SEQ ID NO: 9, 10, 11 or 12 and, optionally, one to twenty amino acids deleted from the C-terminus or N-terminus.

### HCS and PKS Fusion Proteins

Some aspects of the present disclosure are directed to a fusion protein comprising a polypeptide having polyketide synthase activity and a polypeptide having acyl-CoA synthetase activity (e.g., HCS enzymes).

In some embodiments, the polypeptide having polyketide synthase activity comprises an amino acid sequence with at least 70% identity to SEQ ID NO: 1, 2, 3, 4, 5, 6, 7, 8, or 68. In some embodiments, the polypeptide having polyketide synthase activity comprises an amino acid sequence with at least 75% identity to SEQ ID NO: 1, 2, 3, 4, 5, 6, 7, 8, or 68. In some embodiments, the polypeptide having polyketide synthase activity comprises an amino acid sequence with at least 80% identity to SEQ ID NO: 1, 2, 3, 4, 5, 6, 7, 8, or 68. In some embodiments, the polypeptide having polyketide synthase activity comprises an amino acid sequence with at least 85% identity to SEQ ID NO: 1, 2, 3, 4, 5, 6, 7, 8, or 68. In some embodiments, the polypeptide having polyketide synthase activity comprises an amino acid sequence with at least 90% identity to SEQ ID NO: 1, 2, 3, 4, 5, 6, 7, 8 or 68. In some embodiments, the polypeptide having polyketide synthase activity comprises an amino acid sequence with at least 95% identity to SEQ ID NO: 1, 2, 3, 4, 5, 6, 7, 8, or 68. In some embodiments, the polypeptide having polyketide synthase activity comprises an amino acid sequence with at least 99% identity to SEQ ID NO: 1, 2, 3, 4, 5, 6, 7, 8, or 68. In some embodiments, the polypeptide having polyketide synthase activity comprises an amino acid sequence with at least 99.5% identity to SEQ ID NO: 1, 2, 3, 4, 5, 6, 7, 8, or 68. In some embodiments, the polypeptide having polyketide synthase activity is any suitable polypeptide disclosed herein.

In some embodiments, the polypeptide having acyl-CoA synthetase activity (e.g., an HCS) comprises an amino acid sequence with at least 70% identity to SEQ ID NO: 28, 29, 30, 31, 32 or 33. In some embodiments, the polypeptide having acyl-CoA synthetase activity (e.g., an HCS) comprises an amino acid sequence with at least 75% identity to SEQ ID NO: 28, 29, 30, 31, 32 or 33. In some embodiments, the polypeptide having acyl-CoA synthetase activity (e.g., an HCS) comprises an amino acid sequence with at least 80% identity to SEQ ID NO: 28, 29, 30, 31, 32 or 33. In some embodiments, the polypeptide having acyl-CoA synthetase activity (e.g., an HCS) comprises an amino acid sequence with at least 85% identity to SEQ ID NO: 28, 29, 30, 31, 32 or 33. In some embodiments, the polypeptide having acyl-CoA synthetase activity (e.g., an HCS) comprises an amino acid sequence with at least 90% identity to SEQ ID NO: 28, 29, 30, 31, 32 or 33. In some embodiments, the polypeptide having acyl-CoA synthetase activity (e.g., an HCS) comprises an amino acid sequence with at least 95% identity to SEQ ID NO: 28, 29, 30, 31, 32 or 33. In some embodiments, the polypeptide having acyl-CoA synthetase activity (e.g., an HCS) comprises an amino acid sequence with at least 99% identity to SEQ ID NO: 28, 29, 30, 31, 32 or 33. In some embodiments, the polypeptide having acyl-CoA synthetase activity (e.g., an HCS) comprises an amino acid sequence with at least 99.5% identity to SEQ ID NO: 28, 29, 30, 31, 32 or 33.

In some embodiments, the fusion protein further comprises a linker between the polypeptide having polyketide synthase activity and the polypeptide having polyketide cyclase activity. In some embodiments, the linker is between 5 and 52 amino acids in length. In some embodiments, the linker is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52. In some embodiments, the linker has an amino acid sequence selected from SEQ ID NO: 60, 61, or 62.

In some embodiments, the fusion protein comprises the amino acid sequence of SEQ ID NO: 63, 64, or 65. In some embodiments, the fusion protein comprises an amino acid sequence with at least 75%, 80%, 85%, 90%, 95%, 99%, 99.5%, or 99.9% identity to SEQ ID NO: 63, 64, or 65 or a fragment thereof.

In some embodiments, the fusion protein is capable of producing a tetraketide-CoA from Hexanoic acid and/or butyric acid.

In some embodiments, the acyl-CoA synthetase peptide is located at the N-terminus of the polyketide synthase peptide or is connected by a linker to the N-terminus of the polyketide synthase peptide. In some embodiments, the acyl-CoA synthetase peptide is located at the N-terminus of the polyketide synthase or is connected by a linker to the N-terminus of the polyketide synthase.

In some embodiments, the cell comprises a acyl-CoA synthetase as described herein (e.g an amino acid sequence of SEQ ID NO: 28, 29, 30, 31, 32 or 33) and a polyketide synthase as described herein (e.g., an amino acid sequence of SEQ ID NO: 1, 2, 3, 4, 5, 6, 7, or 8) and a polyketide cyclase as described herein (e.g., an amino acid sequence of SEQ ID NO: 9, 10, 11, 12, 69, 70, 71, 72, 73, 74, 75, 76, 77, 78, 79, or 80). In other embodiments the cell comprises a fusion between an acyl-CoA synthetase and a polyketide synthase as described herein (e.g., an amino acid sequence of SEQ ID NO: 63, 64, 65) and a separate polyketide cyclase as described herein (e.g., an amino acid sequence of SEQ ID NO: 9, 10, 11, 12, 69, 70, 71, 72, 73, 74, 75, 76, 77, 78, 79, or 80).

### PKS and PKC Fusion Proteins

Some aspects of the present disclosure are related to a fusion protein comprising a polypeptide having polyketide synthase activity and a polypeptide having polyketide cyclase activity. In some embodiments, the polypeptide having polyketide synthase activity is a polyketide synthase as described herein. In some embodiments, the polypeptide having polyketide synthase activity is a polyketide cyclase as described herein. In some embodiments, the fusion protein comprises a polyketide synthase and polyketide cyclase as described herein.

In some embodiments, the polypeptide having polyketide synthase activity comprises an amino acid sequence with at least 70% identity to SEQ ID NO: 1, 2, 3, 4, 5, 6, 7, 8, or 68. In some embodiments, the polypeptide having polyketide synthase activity comprises an amino acid sequence with at least 75%, 80%, 85%, 90%, 95%, 99%, 99.5%, or 99.9% identity to SEQ ID NO: 1, 2, 3, 4, 5, 6, 7, 8, or 68.

In some embodiments, the polypeptide having polyketide cyclase activity comprises an amino acid sequence with at least 70% identity to SEQ ID NO: 9, 10, 11, 12, 69, 70, 71, 72, 73, 74, 75, 76, 77, 78, 79, or 80. In some embodiments, the polypeptide having polyketide cyclase activity comprises an amino acid sequence with at least 75%, 80%, 85%, 90%, 95%, 99%, 99.5%, or 99.9% identity to SEQ ID NO: 9, 10, 11, 12, 69, 70, 71, 72, 73, 74, 75, 76, 77, 78, 79, or 80.

In some embodiments, the fusion protein further comprises a linker between the polypeptide having polyketide synthase activity and the polypeptide having polyketide cyclase activity. Any suitable linker may be used. In some embodiments, the linker is a polypeptide. In some embodiments, the linker is between 5 and 52 amino acids in length. In some embodiments, the linker comprises, consists of, or consists essentially of the amino acid sequence selected from SEQ ID NOs. 13-27.

In some embodiments, the fusion protein comprises, consists of, or consists essentially the amino acid sequence of SEQ ID NO: 34, 35, 36, 37, 38, or 39. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 34. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 35. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 36. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 37. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 38. In some embodiments, the fusion protein comprises, consists of, or consists essentially an amino acid sequence that has 70%, 75%, 80%, 85%, 90%, 95%, 99%, 99.5%, or 99.9% sequence identity to SEQ ID NO: 39.

In some embodiments, the fusion protein is capable of producing a cyclized polyketide from an acyl-CoA substrate. In some embodiments, the acyl-CoA substrate contains a carboxylic acid that contains two to twenty-two carbons. In other embodiments the Acyl-CoA is selected from the group consisting of acetyl-CoA, Hexanoyl-CoA, octanoyl-CoA, decanoyl-CoA, dodecanoyl-CoA, Palmitoleyl-CoA, Linoleyl-CoA, Palmitoyl-CoA, butyryl-CoA, and Oleyl-CoA. In some embodiments, the fusion protein is capable of producing olivetolic acid from Hexanoyl-CoA. In some embodiments, the fusion protein is capable of producing divarinic acid from Butyryl-CoA. In some embodiments, the fusion protein is capable of producing an OA analog or a DVA analog,

In some embodiments, the fusion protein is capable of producing a ratio of olivetolic acid to olivetol from Hexanoyl-CoA at a ratio of greater than 0.05, 0.06, 0.07, 0.08, 0.09, 0.1, 0.2, 0.3, 0.4, or 0.5. In some embodiments, the fusion protein is capable of producing a ratio of olivetolic acid to olivetol from Hexanoyl-CoA at a ratio of greater than 0.1. In some embodiments, the fusion protein is capable of producing a ratio of divarinic acid to divarinol from Butyryl-CoA at a ratio of greater than 0.05, 0.06, 0.07, 0.08, 0.09, 0.1, 0.2, 0.3, 0.4, or 0.5. In some embodiments, the fusion protein is capable of producing a ratio of divarinic acid to divarinol from Butyryl-CoA at a ratio of greater than 0.1.

In some embodiments, the polypeptide having polyketide synthase activity is located at the N-terminus. In some embodiments, the polypeptide having polyketide cyclase activity is located at the N-terminus.

In some embodiments, a fusion protein described herein comprises a C-terminal and/or an N-terminal scaffolding tag. In some embodiments, the scaffolding tag is capable of forming homodimers. In some embodiments, the scaffolding tag is capable of forming heterodimers. In some embodiments, the scaffolding tag is capable of forming both homodimers and homodimers. In some embodiments, the scaffolding tag has the amino acid sequence of SEQ ID NO: 66 (P3) or 67 (P4). In some embodiments, the fusion protein having a scaffolding tag comprises the amino acid sequence of SEQ ID NO: 46. In some embodiments, the PKC having a scaffolding tag comprises an amino acid sequence having at least about 70%, 75%, 80%, 85%, 90%, 95%, or 99% identity to SEQ ID NO: 46. In some embodiments, a fusion protein having a scaffolding tag is expressed in a cell at a level that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold higher than the level of a fusion protein having the same amino acid sequence but not having a scaffolding tag. In some embodiments, a fusion protein having a scaffolding tag is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold more soluble than a fusion protein having the same amino acid sequence but not having a scaffolding tag. In some embodiments, a fusion protein having a scaffolding tag is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or at least 10-fold more active than a fusion protein having the same amino acid sequence but not having a scaffolding tag.

Some aspects of the present disclosure are directed to a cell comprising a fusion protein disclosed herein. In some embodiments, the cell is a transgenic cell (e.g., the polyketide synthase is coded by a heterologous sequence). In some embodiments, the cell is a yeast cell, in a bacterial cell, in an algae cell, or in a plant cell. In some embodiments, the cell is a yeast cell. In some embodiments, the yeast is an oleaginous yeast (e.g., a Yarrowia lipolytica strain). The cell is not limited and may be any cell disclosed herein.

Some aspects of the present disclosure are directed to a polynucleotide coding for a fusion protein disclosed herein.

An expression vector or vectors can be constructed to include exogenous nucleotide sequences coding for the recombinant polypeptides described herein operably linked to expression control sequences functional in the cell. Any suitable expression vector disclosed herein may be used.

In some embodiments, the cell comprises an exogenous nucleotide sequence coding for a recombinant polypeptide comprising an amino acid sequence having at least 70% identity to SEQ ID NO: 34, 35, 36, 37, 38, or 39. In some embodiments, the cell comprises an exogenous nucleotide sequence coding for a recombinant polypeptide comprising an amino acid sequence having at least 75%, 80%, 85%, 90%, 95%, 99%, 99.5%, or 99.9% identity to SEQ ID NO: 34, 35, 36, 37, 38, or 39.

In some embodiments, the cell comprises an exogenous nucleotide sequence coding for a recombinant polypeptide comprising an amino acid sequence with 1-40 amino acid modifications as compared to SEQ ID NO: 34, 35, 36, 37, 38, or 39 and, optionally, one to twenty amino acids deleted from the C-terminus or N-terminus.

Cells with Improved Cannabinoid and Cannabinoid Precursor Production

Some aspects of the present disclosure are directed to a cell comprising an exogenous nucleotide sequence coding for at least one of the following: (a) a polyketide synthase (e.g., a polyketide synthase described herein) comprising an amino acid sequence with at least 70% identity to SEQ ID NO: 1, 2, 3, 4, 5, 6, 7, 8, or 68, wherein the polyketide synthase has polyketide synthase (PKS) activity; (b) a polyketide cyclase (e.g., a polyketide cyclase described herein) comprising an amino acid sequence with at least 70% identity to SEQ ID NO: 9, 10, 11, 12, 69, 70, 71, 72, 73, 74, 75, 76, 77, 78, 79, or 80, wherein the polyketide cyclase has polyketide cyclase (PKS) activity; (c) a fusion protein (e.g., a fusion protein described herein) comprising a polypeptide having polyketide synthase activity and a polypeptide having polyketide cyclase activity; (d) an enzyme having acyl-CoA synthetase activity and (e) a fusion protein (e.g., a fusion protein described herein) comprising a polypeptide having polyketide synthase activity and a polypeptide having acyl-CoA synthetase activity. In some embodiments, the cell comprises an exogenous nucleotide sequence coding for at least two of (a)-(e). In some embodiments, the cell comprises an exogenous nucleotide sequence coding for at least three of (a)-(e). In some embodiments, the cell comprises an exogenous nucleotide sequence coding for all four of (a)-(d). In some embodiments, the cell comprises (a), (b), and (d). In some embodiments, the cell comprises (a), (c), and (d). In some embodiments, the cell comprises (b), (c), and (d). In some embodiments, the cell comprises (c) and (e), In some embodiments the cell comprises (b) and (e).

The polyketide synthase of (a) is not limited and may be any suitable polyketide synthase described herein. In some embodiments, the polyketide synthase of (a) comprises the amino acid sequence of SEQ ID NO:1, 2, 3, 4, 5, 6, 7, 8, or 68. In some embodiments, the cell comprises (a) a polyketide synthase, wherein the polyketide synthase is capable of producing a tetraketide from one or more acyl-CoA substrates selected from carboxylic acids with two to twenty-two carbons, such as for example Acetyl-CoA, Butyryl-CoA, Hexanoyl-CoA, octanoyl-CoA, decanoyl-CoA, dodecanoyl-CoA, myristoyl-CoA Palmitoleyl-CoA, Linoleyl-CoA, Palmityl-CoA, and Oleyl-CoA.

The polyketide cyclase of (b) is not limited and may be any suitable polyketide cyclase described herein. In some embodiments, the cell comprises (b) a polyketide cyclase, wherein the polyketide cyclase is capable of producing olivetolic acid (OA), an OA analog, divarinic acid (DVA), or a DVA analog from a tetraketide.

The fusion protein of (c) is not limited and may be any suitable fusion protein described herein. In some embodiments, the cell comprises (c) a fusion protein capable of producing olivetolic acid from Hexanoyl-CoA and/or divarinic acid from Butyryl-CoA.

The enzyme having acyl-CoA synthetase activity is not limited and may be any suitable enzyme. In some embodiments, the cell comprises (d) an enzyme having acyl-CoA synthetase activity. In some embodiments, the enzyme has hexanoyl-CoA synthetase (HCS) activity or butyryl-CoA synthetase activity. In some embodiments, the enzyme having HCS activity comprises an amino acid sequence selected from SEQ ID NO: 28, 29, 30, 31, 32, and 33 or an amino acid sequence with at least 70%, 75%, 80%, 85%, 90%, 95%, 99%, 99.5%, or 99.9% identity to SEQ ID NO: 28, 29, 30, 31, 32, or 33.

In some embodiments, the cell comprises a polyketide synthase described herein and a polyketide cyclase described herein. In some embodiments, the cell comprises a polyketide synthase comprising an amino acid sequence with at least 70%, 75%, 80%, 85%, 90%, 95%, 98%, 995, 99.5%, or 99.9% identity to SEQ ID NO:1, 2, 3, 4, 5, 6, 7, or 8 and a polyketide cyclase comprising an amino acid sequence with at least 70%, 75%, 80%, 85%, 90%, 95%, 98%, 99%, 99.5% or 99.9% identity to SEQ ID NO: 9, 10, 11, 12, 69, 70, 71, 72, 73, 74, 75, 76, 77, 78, 79, or 80. In some embodiments, the cell comprises a polyketide synthase comprising an amino acid sequence of SEQ ID NO: 1, 2, 3, 4, 5, 6, 7, or 8 and a polyketide cyclase comprising an amino acid sequence of SEQ ID NO: 9, 10, 11, 12, 69, 70, 71, 72, 73, 74, 75, 76, 77, 78, 79, or 80. In some embodiments, the cell comprises a fusion protein of SEQ ID NO: 34, 35, 36, 37, 38, or 46.

In some embodiments, the cell further comprises an exogenous polynucleotide coding for a chalcone isomerase-like (CHIL) protein heterologous to the cell. CHIL are non-catalytic proteins that are ubiquitous in plant genomes and in this case are thought to interact with CHS to increase its activity and selectivity (Waki T, et al Nature Communications 2020, 11, 870). The CHIL protein is not limited and may be any suitable CHIL protein. In some embodiments, the exogenous CHIL protein increases OA or CBGA titers and reduces the byproducts OL, HTAL, and PDAL. In some embodiment, the CHIL protein is selected from SEQ ID NOs. 49-56.

In some embodiments, the cell is capable of utilizing hexanoic acid to produce olivetolic acid. In some embodiments, the cell is capable of utilizing butyric acid to produce divarinic acid. In some embodiments, the cell is capable of utilizing octanoic acid, decanoic acid, dodecanoic acid, oleic acid, palmitic acid, myristic acid or stearic acid to produce one or more olivetolic acid analogs.

In some embodiments, the cell comprises an upregulated MVA pathway.

In some embodiments, the cell expresses a CBGA synthase, a CBGVA synthase or a synthase that can condense 6-alkyl-2,4-dihydroxy benzoate (FIG. 2 Compound B) with GPP to produce an OA analog.

In some embodiments, the cell is a yeast cell, in a bacterial cell, in an algae cell, or in a plant cell. In some embodiments, the cell is a yeast cell. In some embodiments, the yeast is an oleaginous yeast (e.g., a Yarrowia lipolytica strain). The cell is not limited and may be any suitable cell disclosed herein.

In some embodiments, the cell described herein comprises one or more additional metabolic pathway transgene(s). In some embodiments, the cell comprises an olivetolic acid pathway. In some embodiments, the olivetolic acid pathway comprises a polyketide cyclase. In some embodiments, an exogenous nucleotide codes for the polyketide cyclase. In some embodiments, the olivetolic acid pathway comprises polyketide synthase/olivetol synthase (condensation of hexanoyl coenzyme A (CoA) and 3× malonyl CoAs). In some embodiments, the cell comprises a geranyl pyrophosphate (GPP) pathway. In some embodiments, the GPP pathway comprises geranyl pyrophosphate synthase. In some embodiments, an exogenous nucleotide codes for the geranyl pyrophosphate synthase. In some embodiments, the cell comprises a farnesyl pyrophosphate (FPP) pathway. In some embodiments, the FPP pathway comprises a farnesyl pyrophosphate synthase. In some embodiments, the farnesyl pyrophosphate synthase is a mutant form. In some embodiments, the mutant farnesyl pyrophosphate synthase is described in (Jian G-Z, et al Metabolic Engineering, 2017, 41, 57, incorporated herein). In some embodiments, an exogenous nucleotide codes for the farnesyl pyrophosphate synthase. In some embodiments, the cell comprises a divarinic acid (DVA) pathway. In some embodiments, the DVA pathway comprises divarinic acid synthase. In some embodiments, an exogenous nucleotide codes for the divarinic acid synthase. In some embodiments, the cell comprises a mevalonate pathway. In some embodiments, the cell expresses HMG-CoA reductase. In some embodiments, an endogenous mevalonate pathway of the cell has been manipulated to reduce or increase production of mevalonate, isopentyl pyrophosphate (IPP) or dimethylallyl pyrophosphate (DMAP), geranyl pyrophosphate (GPP) or farnesyl pyrophosphate (FPP). In some embodiments, the cell comprises a polyketide cyclase that produces OA, DVA, and/or derivatives thereof. In some embodiments, the cell comprises a polyketide synthase that produces a tetraketide substrate of the polyketide cyclase. In some embodiments, the cell comprises a polyketide synthase that can directly form OA and derivatives from acetyl-CoA or butyryl-CoA or hexanoyl-CoA and malonyl-CoA.

In some embodiments, the cell is capable of producing a cannabinoid, a cannabinoid derivative, or cannabinoid analogue. The cannabinoids are not limited and may be any cannabinoid described herein. In some embodiments, the cannabinoid is selected from tetrahydrocannabinolic acid, cannabidiolic acid, cannabigerolic acid, or analogue thereof.

In some embodiments, production of the cannabinoid by the cell is under control of a constitutional or inducible promoter. The promoter is not limited and may be any suitable promoter known in the art.

Some aspects of the present disclosure are directed to a composition comprising a cannabinoid, cannabinoid derivative, or cannabinoid analogue produced by a cell disclosed herein. In some embodiments, the composition further comprises a cell as described herein. In some embodiments, the composition comprises purified or isolated cannabinoid, cannabinoid derivative, or cannabinoid analogue produced by a cell disclosed herein. In some embodiments, the composition comprises cannabigerolic acid, tetrahydrocannabinolic acid, cannabidiolic acid, cannabigerolic acid, or derivative, or an analogue thereof.

### Methods of Producing Cannabinoids and Cannabinoid Precursors

Some aspects of the present disclosure are directed to methods of producing olivetolic acid (OA), OA analogs, divarinic acid (DVA), or DVA analogs comprising contacting a transgenic cell as described herein with a fatty acid under suitable conditions to produce the olivetolic acid (OA), OA analogs, divarinic acid (DVA), or DVA analogs. The OA and DVA analogs are not limited. In some embodiments, the OA analogs and DVA analogs are 6-alkyl-2,4-dihydroxy benzoic acid provided in FIG. 2 (Compound B).

Some aspects of the present disclosure are directed to contacting a cell comprising a polyketide synthase as described herein with a fatty acid under suitable conditions to produce a cannabinoid or cannabinoid precursor as described herein. Some aspects of the present disclosure are directed to contacting a cell comprising a polyketide cyclase as described herein with a fatty acid under suitable conditions to produce a cannabinoid or cannabinoid precursor as described herein. Some aspects of the present disclosure are directed to contacting a cell comprising a fusion protein as described herein with a fatty acid under suitable conditions to produce a cannabinoid or cannabinoid precursor as described herein.

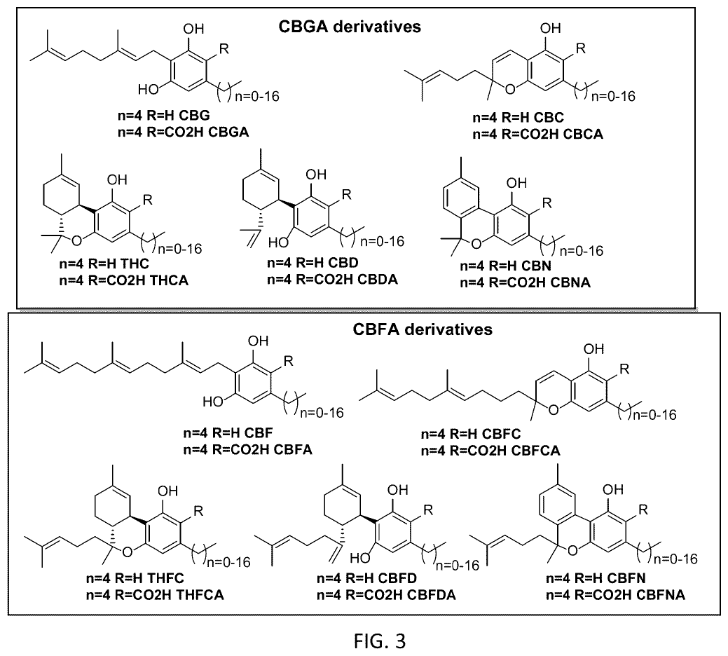

In some embodiments, the cell contacted with a fatty acid has an upregulated MVA pathway and/or expresses CBGA synthase. In some embodiments, the cell contacted with a fatty acid has a synthase that can condense 6-alkyl-2,4-dihydroxy benzoic acid with GPP to produce a CBGA analog (e.g., as shown in FIG. 3). In some embodiments, the cell contacted with a fatty acid has a synthase that can condense 6-alkyl-2,4-dihydroxy benzoic acid with FPP to produce a CBFA analog analog (e.g., as shown in FIG. 3).

In some embodiments, the cell produces one or more of OA, GPP, FPP, and mevalonate (MVA). In some embodiments, the cell produces OA and FPP. In some embodiments, the cell produces OA and GPP. In some embodiments, the cell produces MVA and GPP. In some embodiments, one or more of OA, geraniol, farnesol, prenol, isoprenol, and MVA is provided in a culture medium for use by the cell.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, mannitol, isomaltose, xylose, pannose, maltose, arabinose, cellobiose and 3-, 4-, or 5-oligomers thereof. Other carbon sources include alcohol carbon sources such as methanol, ethanol, glycerol. Other carbon sources include acid and esters such as acetate, formate, fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein.

The culture conditions can include, for example, liquid culture procedures as well as fermentation and other large-scale culture procedures. Useful yields of the products can be obtained under aerobic culture conditions. An exemplary growth condition for achieving, one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration or centrifugal partition chromatography (CPC).

In some embodiments, the cells are grown in stirred tank fermenters with feed supplementation (sugars with or without organic acids) where the dissolved oxygen, temperature, and pH are be controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, Bis(2-ethylhexyl) adipate, decane, dodecane, hexadecane or anther organic solvent with log P>5. The later number (log P) is defined as the log of a compound's partition between water and octanol and is a standard parameter of a compound's hydrophobicity (the larger the log P the less soluble in water). Depending on the fermentation process, the products can be isolated and purified using different methods.

If no organic cosolvent is used different methods can be applied. In one embodiment, insoluble products (CBGA, CBDA, THCA and similar compounds) precipitate together with the cell biomass and the solids are isolated from the liquid supernatant using centrifugation (ultra)filtration or spray drying. The cannabinoid containing cell pellet is then washed with an aqueous miscible organic solvent (ethanol, acetonitrile, etc.) that dissolves the cannabinoid. The soluble cannabinoid solution is then separated by the insoluble cells by filtration. Evaporation of the organic solvent produces an oil that contains the cannabinoid and other oil extracts. In some embodiments the dried cannabinoid cells biomass (produced by spray drying or drying wet cell pellet) can be extracted using supercritical carbon dioxide. Evaporation of the CO2 will produce an oil that contains the extracted cannabinoid in the form of an oil. The cannabinoids from the oils obtained from ethanol extraction or CO2 extraction can be further purified using methods known to the cannabis industry. These can include fractional distillation, crystallization, chromatography or a combination of these techniques. Alternatively, the cell supernatant can be extracted with an aqueous immiscible organic solvent (ethyl acetate, heptane, decane, etc.) to extract the cannabinoids. Evaporation of the organic solvent and produces a cannabis containing oil that can be further purified as described above.

In some embodiments, an organic solvent is required during growth that is separated at the end of the fermentation. Back extraction with basic aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

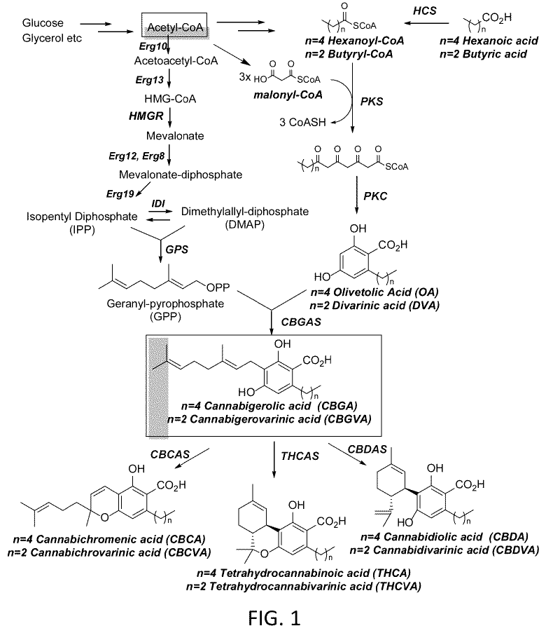

A detailed description of the biosynthesis pathways to all common cannabinoids is shown in FIG. 1. All cells biosynthesize GPP, so an optimized cell would be modified to upregulate this pathway. Various examples of engineered microbial strains (E. coli, yeast, Yarrowia etc) with upregulated mevalonate pathway (MVA) have been published (AA Malico, MA Calzini, AK Gayen, GJ Williams J Ind Microbiol Biotechnol 2020, 47, 675-702). It is noteworthy that although most of the enzymes of the MVA pathway have been altered, i.e over-expressed, mutated, or replaced with enzymes from other organisms, no universal solution that upregulate equally GPP or FPP biosynthesis can be applied to all cells and derived products, although some general rules for its upregulation have been identified (Y. Zu, K L J Prather, G. Stephanopoulos Curr Opin Biotechnol, 2020, 66, 1-8).

In contrast to MVA pathway, enzymes for the biosynthesis of olivetolic acid and some of its analogs are only present in a small number of organisms, mainly plants (T Gulk, BL Møller Trends in Plant Science 2020, 25, 985), while enzymes for the last steps of cannabinoid formation (CBGA, CBGVA etc) have so far only been identified in cannabis and related plants. Consequently, most academic or industrial groups that are developing recombinant organisms (most commonly Saccharomyces cerevisiae yeast and E. coli) for the synthesis of cannabinoids are utilizing the cannabis derived enzymes for the synthesis of OA and CBGA (JD Keasling, et al, Nature 2019, 567, 123-126). For the synthesis of OA, these enzymes are hexanoyl-CoA synthetase (HCS), tetraketide synthase or polyketide synthase (PKS) and olivetolic acid cyclase or polyketide cyclase (PKC) from C. Sativa.

### Example 1: PKS Discovery and Engineering

As described in the general methods below, a number of natural sequences (>40) with potential activity towards the formation of OA or OA analogs were identified, cloned in expression vectors and screened for activity.

Key findings: (1) New non-cannabis polyketide synthases, PKSs, with activity towards producing OA and DVA (when co-expressed with PKC) were identified. (2) Enzymes with higher activity for producing OA and OL were identified. (3) In addition to OA, OA or OL analogs with acyl chains of different lengths can be accessed using these enzymes (FIG. 2). (4) Engineering to improve or alter selectivity of these enzymes in progress, and mutagenesis strategy is described.

The strategy taken to identify polyketide synthases (PKS) with putative OA/OL (with/without PKC present) production activity relied on three general approaches. The first approach involved identifying and selecting sequence homologs of PKS1 (the native Cannabis enzyme with known OA/OL production activity). The second relied on a literature search of enzymes that produce similar products using analogous substrates. The third utilized artificial intelligence methods to identify potential enzymes with activity to produce OA/OL. After this analysis, a total of 33 new enzymes were identified and cloned including PKS1. Each of these was expressed in E. coli, purified, and screened for OL production from hexanoyl-CoA and malonyl-CoA. Based on these results, a subset was screened for activity in Yarrowia whole cells with fed hexanoic acid with PKS23 producing the highest concentration of OL (besides PKS1). Eleven sequence homologs of PKS23 were then identified and screened in Yarrowia whole cells with fed hexanoic acid. A table listing the relative sequence identities shared by the enzymes that produced OL is shown below.

TABLE 1
Relative sequence identities shared by PKSs.

| | PKS1 | PKS23 | PKS34 | PKS35 | PKS36 | PKS37 | PKS40 | PKS41 |
|---|---|---|---|---|---|---|---|---|
| PKS1 | | 48.2 | 47.9 | 48.2 | 48.5 | 49.1 | 48.7 | 49.7 |
| PKS23 | 48.2 | | 86.1 | 85.9 | 91.1 | 81 | 80.7 | 81.2 |
| PKS34 | 47.9 | 86.1 | | 98.7 | 84.3 | 90.5 | 89.5 | 89.5 |
| PKS35 | 48.2 | 85.9 | 98.7 | | 84.1 | 90.5 | 89 | 89.8 |
| PKS36 | 48.5 | 91.1 | 84.3 | 84.1 | | 79.2 | 79.6 | 80.1 |
| PKS37 | 49.1 | 81 | 90.5 | 90.5 | 79.2 | | 87.8 | 88.8 |
| PKS40 | 48.7 | 80.7 | 89.5 | 89 | 79.6 | 87.8 | | 95.2 |
| PKS41 | 49.7 | 81.2 | 89.5 | 89.8 | 80.1 | 88.8 | 95.2 | |

### Screening PKSs for OL Formation In Vivo

As described below, plasmids pCL-SE-0332.PKS1, pCL-SE-0332.PKS34, pCL-SE-0332.PKS35, pCL-SE-0332.PKS36, pCL-SE-0332.PKS37, pCL-SE-0332.PKS40, and pCL-SE-0332.PKS41 were transformed into strain sCL-SE-0041 and three separate colonies each were patched and precultured for 48 h. Assay cultures consisted of YPD medium with 1 mg/mL hygromycin and 2.5 mM butyric, hexanoic, octanoic, lauric, or myristic acid. After 24 h, an additional 10 mM fatty acid was added to each assay culture. Cultures were quenched with equal volume of Ethanol after 48 h total growth and were analyzed by HPLC for product formation. PKS1 produced divarinol or olivetol from fed butyric or hexanoic acid, respectively. PKS34 and PKS37 produced divarinol from fed butyric acid. PKS34-37,40-41 all produced olivetol or olivetol analogs (no PKC is present in the strains) from fed hexanoic, octanoic, lauric, or myristic acid, as well as high concentrations of olivetol analogs with alkyl side chains of different length than the fed fatty acids, likely using fatty acyl-CoAs produced natively in the host. Corresponding PDAL and olivetolic acid analogs were also detected at lower concentrations than the olivetol analogs. Averages and standard deviations were calculated from replicates.

TABLES 2A-2E
Screening of PKSs in Yarrowia sCL-SE-0041 with fed butyric, hexanoic, octanoic, lauric, or myristic
acid. Products in μM accumulated in the in vivo assay. The C2 olivetol analog was detected but could
not be quantified during the fed hexanoic acid experiments so is not listed. PDAL and olivetolic acid
analogs were detected for all olivetol analog products but at lower concentrations. Peaks of the C16
and C18:1 olivetol analogs overlapped during the fed octanoic acid experiments, so concentrations
are listed as a total of both molecules. All concentrations of products are reported in μM.
Butyric Acid Feed (Table 2A)

| | | Acetyl- | Palmitoleyl- | Linoleyl- | Palmityl- | |
|---|---|---|---|---|---|---|
| | Butyryl- | CoA | CoA | CoA | CoA | Oleyl-CoA |
| Plasmid in | CoA | C2 | 16:1 | 18:2 | 16 | 18:1 |
| sCL-SE-0041 | Divarinol | Olivetol | Olivetol | Olivetol | Olivetol | Olivetol |
| pCL-SE- | 28 ± 2 | 0 ± 0 | 0 ± 0 | 0 ± 0 | 0 ± 0 | 0 ± 0 |
| 0332.PKS1 | | | | | | |
| pCL-SE- | 3 ± 1 | 705 ± 29 | 648 ± 41 | 106 ± 9 | 503 ± 50 | 370 ± 41 |
| 0332.PKS34 | | | | | | |
| pCL-SE- | 0 ± 0 | 687 ± 9 | 488 ± 25 | 67 ± 4 | 358 ± 23 | 252 ± 17 |
| 0332.PKS35 | | | | | | |
| pCL-SE- | 0 ± 0 | 388 ± 7 | 557 ± 2 | 66 ± 0 | 159 ± 3 | 246 ± 1 |
| 0332.PKS36 | | | | | | |
| pCL-SE- | 7 ± 0 | 738 ± 22 | 750 ± 16 | 135 ± 2 | 585 ± 10 | 464 ± 11 |
| 0332.PKS37 | | | | | | |
| pCL-SE- | 0 ± 0 | 527 ± 17 | 304 ± 11 | 40 ± 1 | 156 ± 7 | 127 ± 4 |
| 0332.PKS40 | | | | | | |
| pCL-SE- | 0 ± 0 | 543 ± 20 | 368 ± 13 | 45 ± 1 | 169 ± 3 | 165 ± 4 |
| 0332.PKS41 | | | | | | |

Hexanoic Acid Feed (Table 2B)

| | | | | Palmitoleyl- | Linoleyl- | Palmityl- | |
|---|---|---|---|---|---|---|---|
| | | | | CoA | CoA | CoA | |
| Plasmid in | Hexanoyl-CoA | | | 16:1 | 18:2 | 16 | 18:1 |
| sCL-SE-0041 | PDAL | OA | Olivetol | Olivetol | Olivetol | Olivetol | Olivetol |
| pCL-SE- | 31 ± 6 | 3 ± 0 | 172 ± 20 | 0 ± 0 | 0 ± 0 | 0 ± 0 | 0 ± 0 |
| 0332.PKS1 | | | | | | | |
| pCL-SE- | 32 ± 7 | 5 ± 1 | 264 ± 59 | 498 ± 85 | 119 ± 21 | 304 ± 42 | 368 ± 66 |
| 0332.PKS34 | | | | | | | |
| pCL-SE- | 24 ± 6 | 3 ± 1 | 160 ± 33 | 330 ± 87 | 60 ± 21 | 159 ± 47 | 191 ± 70 |
| 0332.PKS35 | | | | | | | |
| pCL-SE- | 11 ± 1 | 0 ± 0 | 34 ± 6 | 442 ± 11 | 80 ± 3 | 103 ± 5 | 240 ± 11 |
| 0332.PKS36 | | | | | | | |
| pCL-SE- | 34 ± 9 | 6 ± 1 | 327 ± 86 | 447 ± 143 | 123 ± 41 | 303 ± 110 | 369 ± 146 |
| 0332.PKS37 | | | | | | | |
| pCL-SE- | 20 ± 7 | 1 ± 1 | 48 ± 14 | 206 ± 39 | 29 ± 9 | 94 ± 28 | 109 ± 48 |
| 0332.PKS40 | | | | | | | |
| pCL-SE- | 14 ± 2 | 2 ± 1 | 46 ± 6 | 205 ± 57 | 28 ± 5 | 67 ± 24 | 102 ± 38 |
| 0332.PKS41 | | | | | | | |

Octanoic Acid Feed (Table 2C)

| | | | | Linoleyl- | |
|---|---|---|---|---|---|
| | | | | CoA/Palmityl- | |
| | Octanoyl- | Acetyl- | Palmitoleyl- | CoA | |
| | CoA | CoA | CoA | 18:2 | Oleyl-CoA |
| Plasmid in | C8 | C2 | 16:1 | Olivetol/16 | 18:1 |
| sCL-SE-0041 | Olivetol | Olivetol | Olivetol | Olivetol | Olivetol |
| pCL-SE- | 0 ± 0 | 0 ± 0 | 0 ± 0 | 0 ± 0 | 0 ± 0 |
| 0332.PKS1 | | | | | |
| pCL-SE- | 124 ± 14 | 301 ± 79 | 494 ± 46 | 309 ± 35 | 292 ± 22 |
| 0332.PKS34 | | | | | |
| pCL-SE- | 88 ± 3 | 203 ± 26 | 356 ± 34 | 223 ± 12 | 182 ± 19 |
| 0332.PKS35 | | | | | |
| pCL-SE- | 36 ± 1 | 257 ± 21 | 552 ± 10 | 250 ± 20 | 255 ± 18 |
| 0332.PKS36 | | | | | |
| pCL-SE- | 92 ± 2 | 0 ± 0 | 142 ± 4 | 132 ± 6 | 98 ± 6 |
| 0332.PKS37 | | | | | |
| pCL-SE- | 26 ± 0 | 98 ± 11 | 190 ± 30 | 100 ± 6 | 84 ± 8 |
| 0332.PKS40 | | | | | |
| pCL-SE- | 35 ± 0 | 44 ± 0 | 85 ± 0 | 41 ± 0 | 39 ± 0 |
| 0332.PKS41 | | | | | |

Lauric Acid Feed (Table 2D)

| | Lauroyl- | Acetyl- | Palmitoleyl- | Linoleyl- | Palmityl- | Oleyl- |
|---|---|---|---|---|---|---|
| | CoA | CoA | CoA | CoA | CoA | CoA |
| Plasmid in | C12 | C2 | 16:1 | 18:2 | 16 | 18:1 |
| sCL-SE-0041 | Olivetol | Olivetol | Olivetol | Olivetol | Olivetol | Olivetol |
| pCL-SE- | 0 ± 0 | 0 ± 0 | 0 ± 0 | 0 ± 0 | 0 ± 0 | 0 ± 0 |
| 0332.PKS1 | | | | | | |
| pCL-SE- | 268 ± 31 | 314 ± 64 | 315 ± 22 | 93 ± 5 | 258 ± 25 | 232 ± 23 |
| 0332.PKS34 | | | | | | |
| pCL-SE- | 200 ± 22 | 293 ± 24 | 272 ± 15 | 55 ± 20 | 199 ± 13 | 183 ± 9 |
| 0332.PKS35 | | | | | | |
| pCL-SE- | 163 ± 15 | 201 ± 6 | 301 ± 14 | 92 ± 2 | 163 ± 3 | 178 ± 7 |
| 0332.PKS36 | | | | | | |
| pCL-SE- | 356 ± 109 | 331 ± 69 | 339 ± 31 | 99 ± 4 | 250 ± 27 | 257 ± 18 |
| 0332.PKS37 | | | | | | |
| pCL-SE- | 120 ± 6 | 297 ± 51 | 222 ± 3 | 44 ± 4 | 123 ± 21 | 119 ± 7 |
| 0332.PKS40 | | | | | | |
| pCL-SE- | 124 ± 16 | 186 ± 39 | 170 ± 21 | 88 ± 20 | 104 ± 29 | 81 ± 7 |
| 0332.PKS41 | | | | | | |

Myristic Acid Feed (Table 2E)

| | Myristoyl- | Acetyl- | Palmitoleyl- | Linoleyl- | Palmityl- | Oleyl- |
|---|---|---|---|---|---|---|
| | CoA | CoA | CoA | CoA | CoA | CoA |
| Plasmid in | C14 | C2 | 16:1 | 18:2 | 16 | 18:1 |
| sCL-SE-0041 | Olivetol | Olivetol | Olivetol | Olivetol | Olivetol | Olivetol |
| pCL-SE- | 0 ± 0 | 0 ± 0 | 0 ± 0 | 0 ± 0 | 0 ± 0 | 0 ± 0 |
| 0332.PKS1 | | | | | | |
| pCL-SE- | 2152 ± 102 | 855 ± 36 | 610 ± 48 | 121 ± 10 | 506 ± 38 | 392 ± 32 |
| 0332.PKS34 | | | | | | |
| pCL-SE- | 2029 ± 30 | 835 ± 10 | 452 ± 7 | 81 ± 9 | 369 ± 26 | 266 ± 10 |
| 0332.PKS35 | | | | | | |
| pCL-SE- | 1929 ± 106 | 676 ± 20 | 495 ± 10 | 111 ± 4 | 356 ± 18 | 288 ± 6 |
| 0332.PKS36 | | | | | | |
| pCL-SE- | 2517 ± 72 | 936 ± 12 | 697 ± 45 | 149 ± 4 | 596 ± 18 | 484 ± 11 |
| 0332.PKS37 | | | | | | |
| pCL-SE- | 1373 ± 62 | 782 ± 53 | 304 ± 7 | 60 ± 4 | 249 ± 3 | 131 ± 2 |
| 0332.PKS40 | | | | | | |
| pCL-SE- | 1488 ± 116 | 783 ± 21 | 344 ± 4 | 60 ± 16 | 272 ± 4 | 176 ± 6 |
| 0332.PKS41 | | | | | | |

sCL-SE-0041 does not have polyketide synthase (PKS) activity. However. the plasmids in this example contain expression cassettes that encode for different PKS genes and the HCS2 gene. HCS2 improves activation of supplemented hexanoic acid to hexanoyl-CoA which is a substrate for some OL producing PKSs.

In the above table, all PKSs examined enabled OL production from hexanoic acid supplementation. In addition, the PKS 34-37 and 40-41 produced polyketides with different length alkyl chains that correspond to the native fatty acids reported to be produced by Yarrowia species (C2, C16, C18, C18:1, C18:2; P. Xu, K. Qiao, W. S Ahn, and G Stephanopoulos PNAS 2016 113 (39) 10848-10853). These PKSs can therefore be used for the synthesis of OL analogs (Compound A, FIG. 2) using fatty acids with different length alkyl chains as substrates such as, but not limited to: acetic (C2 N=0 FIGS. 1 and 2), palmitoleic (C16:1 N=14 FIGS. 1 and 2), linoleic (C18:2 N=16 FIGS. 1 and 2), palmitic (C16, N=14 FIGS. 1 and 2), oleic (C18:1 N=16 FIGS. 1 and 2).

Only olivetol analogs are synthesized in this experiment because the Yarrowia strain (sCL-SE-0041) used for this screen does not contain polyketide cyclase activity (PKC) (FIG. 2). It is well established, that in the absence of PKC (or in inadequate amounts), the tetraketide intermediate is chemically converted to olivetol. In addition to these products, pentyl diacetic lactone (PDAL) can also form from the triketide (FIG. 2). As a result, the formation of OL analogs represents each PKSs selectivity towards different acyl-CoAs and highlights their ability to produce the correct tetraketide precursor for PKC catalyzed cyclization. In the presence of the appropriate cyclase, these PKSs produce the corresponding OA analogs as shown in FIG. 2, Compound B. The later compounds can be condensed with GPP or FPP using the appropriate prenyltransferases to form the compounds shown in FIG. 3.

This example shows that strains expressing some of the PKSs discovered herein produce higher amounts of OL (compared to PKS1 from Cannabis) in the presence of hexanoic acid, which should translate to higher amounts of OA in the presence of a PKC. An example of testing their activity in vivo with PKC1.1 is shown below Furthermore, these enzymes enable the production of OL analogs, which can enable the production of cannabinoid analogs, from a diverse set of acyl-CoAs including long chain fatty acids and other fatty acid analogs as shown in Example 4.

### Example 2: Screening PKSs for OA Formation in the Presence of PKCs with Hexanoic Acid Feed

As described below, plasmids pCL-SE-0332.PKS1, pCL-SE-0332.PKS23, pCL-SE-0332.PKS34, pCL-SE-0332.PKS35, pCL-SE-0332.PKS36, pCL-SE-0332.PKS37, pCL-SE-0332.PKS40, and pCL-SE-0332.PKS41 were transformed into strain SB-0665, which expresses PKC1.1 and HCS2, and four separate colonies each were patched and precultured for 48 h. Assay cultures consisted of YPD media with 1 mg/mL hygromycin and 2.5 mM hexanoic acid. After 24 h, an additional 10 mM hexanoic acid was added to each assay culture. Cultures were quenched after 48 h total growth. Enzymes produced olivetolic acid, olivetol, and PDAL.

TABLE 3
Screening of PKSs in Yarrowia SB-0665 with
fed hexanoic acid. Products in μM accumulated in the in vivo assay.

| | PDAL | OA | Olivetol |
|---|---|---|---|
| Strain | (μM) | (μM) | (μM) |
| SB-0665 + pCL-SE-0332.PKS1 | 36 ± 3 | 51 ± 7 | 50 ± 9 |
| SB-0665 + pCL-SE-0332.PKS23 | 19 ± 1 | 23 ± 2 | 22 ± 5 |
| SB-0665 + pCL-SE-0332.PKS34 | 34 ± 1 | 105 ± 4 | 71 ± 3 |
| SB-0665 + pCL-SE-0332.PKS35 | 26 ± 1 | 64 ± 4 | 45 ± 1 |
| SB-0665 + pCL-SE-0332.PKS36 | 17 ± 0 | 14 ± 0 | 15 ± 3 |
| SB-0665 + pCL-SE-0332.PKS37 | 46 ± 1 | 182 ± 7 | 112 ± 2 |
| SB-0665 + pCL-SE-0332.PKS40 | 23 ± 1 | 25 ± 1 | 23 ± 2 |
| SB-0665 + pCL-SE-0332.PKS41 | 22 ± 1 | 34 ± 2 | 27 ± 4 |

When a PKC was present, all PKSs produced olivetolic acid in addition to olivetol, showing that the PKSs' product is the linear tetraketide molecule that can then be cyclized by the PKC. In addition, PKS34 and PKS37 produced significantly higher amounts of OA compared to PKS1.

### Example 3: Mutagenesis Strategy of PKSs

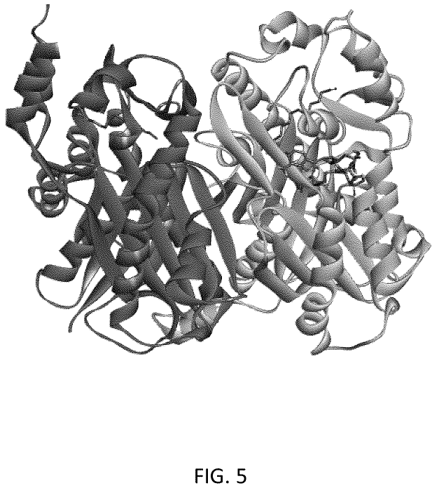

Mutagenesis of the discovered PKSs can be performed to improve selectivity towards specific OL and OA analogs, as well as to improve the enzyme's kinetic properties (K_{M} & k_{cat}) for a specified substrate. For example, engineering can be performed to reduce the PKSs selectivity towards larger fatty acids and increase hexanoyl-CoA activity. Engineering of these enzyme begins by creating structural models as described in Section F of the general techniques (FIG. 5). All amino acids around the substrate's entrance to the active site and all amino acids round the active site can be targeted for mutagenesis. Since these are homodimeric proteins, selected amino acids in the dimer interface will also be mutated (some are assumed to interact with the active site). Mutants will be screened as described herein.

Because of the high homology between all of these PKSs (Table 1) the same mutations that improve activity and selectivity should be transferable to all proteins.

TABLE 4
Mutagenesis sites for each PKS enzyme

| PKS23 | PKS34 | PKS35 | PKS36 | PKS37 | PKS40 | PKS41 |
|---|---|---|---|---|---|---|
| A106 | A102 | A102 | A108 | A103 | A103 | A103 |
| Y140 | Y136 | Y136 | Y142 | Y136 | Y136 | Y137 |
| S141 | S137 | S137 | S143 | S137 | S137 | S138 |
| A145 | A141 | A141 | A147 | A141 | A141 | A142 |
| L169 | L165 | L165 | L171 | L165 | L165 | L166 |
| G171 | G167 | G167 | G173 | G167 | G167 | G168 |
| C172 | C168 | C168 | C174 | C168 | C168 | C169 |
| E200 | E196 | E196 | E202 | E196 | E196 | E197 |
| T202 | T198 | T198 | T204 | T198 | T198 | T199 |
| I204 | I200 | I200 | I206 | I200 | I200 | I201 |
| A205 | A201 | A201 | A207 | A201 | A201 | A202 |
| G208 | G204 | G204 | G210 | G204 | G204 | G205 |
| G219 | G215 | G215 | G221 | G215 | G215 | G216 |
| F223 | F219 | F219 | F225 | F219 | F219 | F220 |
| G224 | G220 | G220 | G226 | G220 | G220 | G221 |
| D225 | D221 | D221 | D227 | D221 | D221 | D222 |
| G226 | G222 | G222 | G228 | G222 | G222 | G223 |
| I263 | I258 | I258 | I264 | I258 | I258 | I259 |
| M265 | M260 | M260 | M266 | M260 | M260 | M261 |
| M272 | M267 | M267 | M273 | M267 | M267 | M268 |
| Y274 | Y269 | Y269 | Y275 | Y269 | Y269 | Y270 |
| H313 | H308 | H308 | H314 | H309 | H309 | H309 |
| G315 | G310 | G310 | G316 | G311 | G311 | G311 |
| N346 | N341 | N341 | N347 | N342 | N342 | N342 |
| S348 | S343 | S343 | S349 | S344 | S344 | S344 |
| F382 | F377 | F377 | F383 | F379 | F379 | F376 |
| G383 | G378 | G378 | G384 | G380 | G380 | G377 |
| P384 | P379 | P379 | P385 | P381 | P381 | P378 |

### PKC Discovery and Engineering

The final amount of OA (or OA analogs—Compound B FIG. 2) as well as the presence of byproducts HTAL and OL (FIG. 2) is defined by the activity of the final cyclase, PKC. To discover cyclases with increased activity towards OA (and analogs) the inventors first cloned and screened several natural sequences, followed by protein engineering to improve the activity and selectivity of selected protein templates.

As described in the general methods below, 37 natural sequences with potential activity towards the formation of OA (or OA analogs) were identified. The enzymes were first expressed in E. coli, purified, and were assayed in vitro for OA formation in the presence of PKS1 and hexanoyl-CoA. Only one enzyme, PKC4, formed detectable amounts of OA. In a first step, a library of chimeric sequences was made by transferring sequence elements of PKC1 to PKC4 as described in the following examples. This method created one chimera, PKC4.8, that was used as a template for further mutagenesis.

### Key Findings

(1) A natural, PKC4 with activity towards OA formation using hexanoic and fatty acids with different carbon chains (where PKS1 was not active) was discovered. Mutagenesis positions to tune the enzyme towards certain OA analogs are also disclosed.

(2) A novel non-natural sequence, PKC4.8 (derived from PKC4), with good activity towards OA formation was developed. Mutagenesis of this enzyme to improve its activity further towards OA and DVA is ongoing, yet enzymes with improved activity have already been discovered and are identified as PKC4.11, PKC4.15, PKC4.17, PKC4.19 PKC4.30-4.33, and PKC4.35-38 (Example 7c) (i.e. SEQ ID NOs 69-80)

(3) Fusions of PKS-PKC were successful in improving OA titers and OA/OL ratio.

(4) Fusion with ubiquitin of PKC1/4.8 at N-terminal OR “zipping” N- and C-terminal to improve enzyme expression and stability have produced enzymes with better activity and OA/OL selectivity

(5) Combination of PKS discovered herein (such as PKS23), with PKC4 and PKC4.8 show that all OA analogs shown in FIG. 2 (and as result cannabinoids of FIG. 3) can be accessed with these enzymes.

### Example 4: In Vitro Screening of Purified PKCs

All enzymes described in this experiment were recombinantly expressed in E. coli and then purified as described in Experimental. In vitro reactions were performed by mixing purified selected PKSs and PKCs (final concentration 2.5 μM and 25 μM respectively) with selected acyl-CoAs (250 μM) and malonyl-CoA (750 μM). In this assay the addition of PKSs was required to synthesize the tetraketide substrates that were then converted to OA and OL products. The reactions were incubated for 60 or 100 min at 30 C before the samples were quenched with equal volume of ethanol and were analyzed for products using HPLC/MS as described in the analytical methods.

TABLE 5
Reactions of selected PKSs with PKCs
using Hexanoyl-CoA. Products in μM
accumulated in the in vitro assay.

| | | | Rxn |
|---|---|---|---|
| | OA | OL | Time |
| Enzyme(s) in Reaction | (μM) | (μM) | (min) |
| PKS1 | ND | 16.1 | 100 |
| PKS23 | ND | 29.6 | 100 |
| PKS1 + PKC4 | 1.2 | 11.5 | 100 |
| PKS23 + PKC4 | 4.4 | 28.3 | 100 |
| PKS23 + PKC4.8 | 27.1 | 3.0 | 100 |
| PKS1 + PKC1 | 6.9 | 1.5 | 60 |
| PKS1 + PKC4.8 | 6.2 | 1.8 | 60 |
| PKS23 + PKC1 | 6.3 | 1.3 | 60 |
| PKS23 + PKC4.8 | 10.0 | 2.1 | 60 |

The above results show that PKC4 and PKC4.8 can synthesize OA with either PKS1 or PKS23. The combination of PKS and PKC affects product ratio as can be seen by the difference in the OA titers between PKS1/PKC4.8 and PKS23/PKC4.8.

Screening of PKC4 and PKC4.8 with different fatty acids: The possibility of making a large variety of OA analogs that are shown in FIG. 2 was tested using PKS23 with PKC4 or PKC4.8. This experiment was performed as described above, with the only difference that several acyl-CoAs (C4 butyryl, C6 hexanoyl, C8 octanoyl, C10 decanoyl, C14 myristyl- and C16 palmityl-CoA) were tested as substrates for the synthesis of the corresponding OL and OA (Table 6).

TABLE 6
Reactions of PKS23 with PKC4/4.8 using C4—C16 acyl-CoAs
(HTAL and PDAL analogs were made in all reactions at lower
concentrations-data not shown).
Products in μM accumulated in the in vitro assay.

| | PKS23 + PKC4 | | PKS23 +PKC4.8 | |
|---|---|---|---|---|
| | OL | OA | OL | OA |
| | Analog | Analog | Analog | Analog |
| Substrate | (μM) | (μM) | (μM) | (μM) |
| Butyryl-CoA | ND | 0.4 | ND | 0.4 |
| Hexanoyl-CoA | 6.0 | 0.9 | 2.1 | 10.0 |
| Octanoyl-CoA | 24.5 | 4.2 | 12.3 | 8.4 |
| Decanoyl-CoA | 12.0 | 7.5 | 18.2 | 1.4 |
| Myristyl-CoA | ND | 5.5 | 3.6 | 1.5 |
| Palmityl-CoA | ND | 1.9 | 0.2 | 0.7 |

By looking at the total products formed in each reaction (OA+OL) it appears that PKS23 is more selective for C6-C14 acyl-CoAs, with strong preference for octanoic (C8) and decanoic (C10). On the other hand, PKC4 shows preference for tetraketides from C8 to C14 fatty acids, while PKC4.8 prefers C6-C8 (and C4 as shown in the in vivo assays of Example 5) fatty acids. In conclusion, the above results show that all OA analogs described in FIG. 2 (Compound B) can be accessed by combining the PKSs described herein (or mutants) with the appropriate PKC4/4.8 (or other mutants). Addition of an appropriate prenyltransferase and terminal cyclase can give access to all cannabinoids described in FIG. 3.

### Example 5: In Vivo Assay of PKCs in Yarrowia Containing PKS1

As described in detail below, plasmids pCL-SE-0331, pCL-SE-0331.PKC1, pCL-SE-0331.PKC4, and pCL-SE-0331.PKC4.8 were transformed into strain SB-0109, which expresses PKS1 and HCS2, and four separate colonies from each transformation were patched and precultured for 48 h. Assay cultures consisted of YPD media with 1 mg/mL hygromycin and 2.5 mM hexanoic acid. After 24 h, an additional 10 mM hexanoic acid was added to each assay culture. Cultures were quenched with equal volume of ethanol after 48 h total growth and were analyzed by HPLC-MS. Enzymes produced olivetol and olivetolic acid. Averages and standard deviations were calculated from replicates. This experiment can be done with alternative PKSs expressed in the strain and/or with fatty acids of different chain lengths (butyric acid, octanoic acid, decanoic acid, etc.).

TABLE 7
Screening of PKCs in Yarrowia SB-00109; hexanoic acid feed.
Products in μM accumulated in the in vivo assay.

| | HTAL | PDAL | OA | OL |
|---|---|---|---|---|
| Strain | (μM) | (μM) | (μM) | (μM) |
| SB-0109 + pCL-SE-0331.PKC1 | 11 ± 0 | 72 ± 1 | 303 ± 5 | 99 ± 3 |
| SB-0109 + pCL-SE-0331.PKC4 | 13 ± 0 | 84 ± 14 | 7 ± 1 | 458 ± 43 |
| SB-0109 + pCL-SE-0331.PKC4.8 | 12 ± 0 | 79 ± 3 | 191 ± 7 | 233 ± 8 |
| SB-0109 + pCL-SE-0331 | 13 ± 0 | 81 ± 5 | 6 ± 0 | 450 ± 16 |

TABLE 7a
Screening of PKCs in Yarrowia SB-00109; butyric acid feed.
Products in μM accumulated in the in vivo assay. HTAL and PDAL
analogs were detected at low levels but not quantified.

| Strain | DVA (μM) | Divarinol (μM) |
|---|---|---|
| SB-0109 + pCL-SE-0331.PKC1 | 44 ± 5 | 7 ± 2 |
| SB-0109 + pCL-SE-0331.PKC4 | 0 ± 0 | 42 ± 1 |
| SB-0109 + pCL-SE-0331.PKC4.8 | 29 ± 2 | 25 ± 2 |
| SB-0109 + pCL-SE-0331 | 0 ± 0 | 45 ± 3 |

In agreement with the in vitro results, PKC4 has very low activity for hexanoyl-CoA in vivo. Similarly, PKC4.8, produces roughly 30-fold more olivetolic acid compared to PKC4 and almost two thirds that of PKC1 in the presence of PKS1.

The novel PKC4.8 discovered herein shows good activity for OA and Divarinic acid (DVA). Further mutagenesis (described below) will increase its activity towards OA and DVA. Mutants with selectivity for other OA analogs (N=6-16, FIG. 2) will be developed. Furthermore, combination of PKC4.8 and selected mutants with the PKSs discovered herein (capable of accepting fatty acids with longer chains) will give access to OA analogs shown in FIG. 2 (N=6-16).

### Example 6: Fusion of PKC1.1 and PKS1 and In Vivo Screening

This example shows that fusion proteins comprised of PKS and PKC can improve both the titers of OA and the OA/OL ratio. To optimize the activity of fused enzymes, numerous linker sequences were evaluated based on length and flexibility.

As described below, plasmids pCL-SE-0476 to 0481 were transformed into strain SB-0109. For each transformation, four separate colonies were patched and precultured for 24 h. Assay cultures consisted of 500 μL YPD buffered with 100 mM MES pH 6.5 containing 1 mg/mL hygromycin and 10 mM hexanoic acid. After 24 h the cultures were quenched with equal volume of ethanol and were analyzed for olivetol and olivetolic acid by HPLC-MS. Averages and standard deviations were calculated from replicates. This experiment can be done with alternative PKSs expressed in the strain. The results are shown below (Table 8):

TABLE 8
Screening of PKS1-PKC1 fusions in Yarrowia strains lacking
PKC activity. Products in μM accumulated in the in vivo assay.

| | OA | OL | |
|---|---|---|---|
| Strain | (μM) | (μM) | OA/OL |
| SB-0109 + pCL-SE-0016 | 0 ± 0 | 37 ± 1 | 0.0 |
| SB-0109 + pCL-SE-0476 | 20 ± 0 | 36 ± 1 | 0.6 |
| SB-0109 + pCL-SE-0477 | 26 ± 1 | 36 ± 1 | 0.7 |
| SB-0109 + pCL-SE-0478 | 11 ± 0 | 37 ± 1 | 0.3 |
| SB-0109 + pCL-SE-0479 | 7 ± 2 | 34 ± 1 | 0.2 |
| SB-0109 + pCL-SE-0480 | 4 ± 0 | 39 ± 2 | 0.1 |
| SB-0109 + pCL-SE-0481 | 12 ± 0 | 36 ± 2 | 0.3 |

The above table show that when SB-0109 is transformed with the empty vector it cannot produce olivetolic acid and as expected, accumulates olivetol. The PKC-PKS fusions enable SB-0109 to produce olivetolic acid (pCL-SE-0476, 0477). The fusions with PKS at the N-terminus (pCL-SE-0487-0481) were much less active but still produced some OA product. The fusion with linker F2 (pCL-SE-0477) produced the highest levels of olivetolic acid and the best OA to OL ratio.

As described Experimental Section, the fusion construct, pCL-SE-0477 and the vector control, pCL-SE-0016 were transformed into SB-0264 to see if it can improve both olivetolic acid production and the ratio of olivetolic acid to olivetol. For each transformation, eight separate colonies were patched and precultured for 24 h. Assay cultures consisted of YPD buffered with 100 mM MES pH 6.5 containing 1 mg/ml hygromycin and 10 mM hexanoic acid. After 24 h the cultures were quenched and analyzed for olivetol and olivetolic acid. Averages and standard deviations were calculated from replicates. This experiment can be done with alternative PKSs expressed in the strain. The results are shown below (Table 9):

TABLE 9
In vivo screening of PKS1-PKC1 fusions in Yarrowia strains
with PKC1/PKS1 background. Products in μM accumulated
in the in vivo assay.

| | OA | OL | |
|---|---|---|---|
| Strain | (μM) | (μM) | OA/OL |
| SB-0264 + pCL-SE-0016 | 17 ± 1 | 19 ± 1 | 0.9 |
| SB-0264 + pCL-SE-0477 | 48 ± 2 | 25 ± 1 | 1.9 |

These data show that when strain SB-0264 is transformed with the empty vector it produces slightly more olivetol than olivetolic acid. However, when it is transformed with the PKC-PKS fusion there is a marked improvement in olivetolic acid formation and an improvement in the ratio of olivetolic acid to olivetol. Both results are advantageous for producing cannabinoids since olivetolic acid is the desired product of this reaction as it, not olivetol, is a key precursor for cannabinoid formation.

This example shows that all PKS-PKC fusions were active in Yarrowia, and fusions with linker F2 produced both the highest amount of OA and the best OA/OL ratio.

### Example 7: N- and C-Terminal Modification of PKC1 and PKC4.8

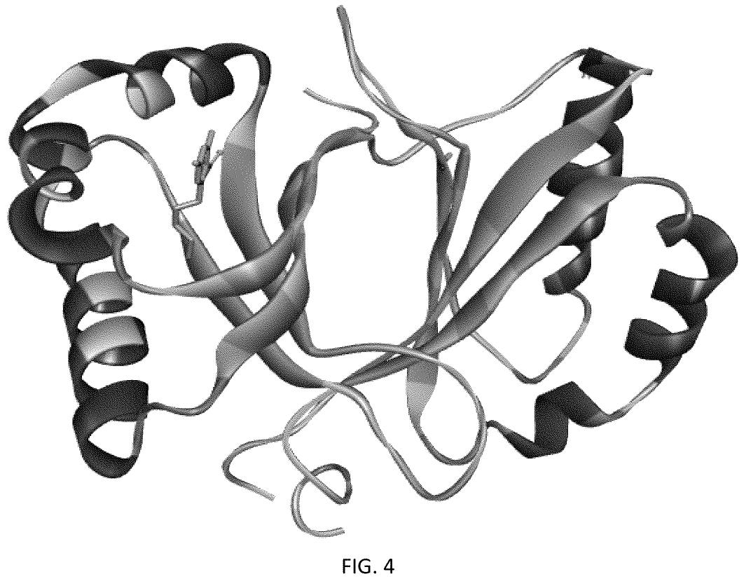

Low expression of most polyketide cyclases (PKCs) was observed in E. coli (most PKCs produced inclusion bodies) and low soluble expression was also observed in Yarrowia as evaluated by western blots. To increase the solubility and therefore the expression of PKC1, PKC4 and PKC4.8 the enzymes were fused at the N-terminal to ubiquitin (See, e.g., SEQ ID NOs. 43, 58, and 59). In one alternative strategy both the N- and C-terminus were fused to a small peptide that can dimerize. The later method may provide further stabilization of the PKCs by dimerizing or “zipping” the N- and C-terminus of the protein as shown in FIG. 4. In a second strategy, a ubiquitin tag is introduced at the N-terminus to potentially improve expression and solubility. A third strategy attempts to cluster PKC and PKS together using a combination of scaffolding and fusion. In this approach, a P3 and P4 scaffolding domains are used. These domains interact with each other; thus, an PKC dimer that has an N-terminal P3 domain may interact with a PKC dimer that has a C-terminal P4 domain. Furthermore, the P3-domain is introduced at the N-terminus of the PKC-PKS fusion such that a scaffold of PKC dimers could be formed around the PKS of the PKC-PKS fusion. Another strategy is to truncate the N- and/or C-terminus.

TABLE 10
Enzyme modifications made for
testing expression and activity

| Enzyme ID | PKC | Modification |
|---|---|---|
| PKC1_Zip_1 | PKC1 | N-Zip_1N, C-Zip_1C |
| PKC1_Zip_2 | PKC1 | N-Zip_1N, C-Zip_2C |
| PKC4.8_Zip_1 | PKC4.8 | N-Zip_1N, C-Zip_1C |
| PKC1.1_Ub_1 | PKC1.1 | N-terminal Ubiquitin |
| PKC4_Ub_1 | PKC4 | N-terminal Ubiquitin |
| PKC4.8_Ub_1 | PKC4.8 | N-terminal Ubiquitin |
| P3-PKC1.1 | PKC1.1 | N-terminal P3 scaffold domain |
| PKC1.1-P4 | PKC1.1 | C-terminal P4 scaffold domain |
| P3-PKC1.1-PKS1 | PKC1.1 | N-terminal P3 scaffold domain |
| Zip_1N | | N-terminal sequence-linker1 |
| Zip_1C | | C-terminal sequence-linker1 |
| Zip_2C | | C-terminal sequence-linker2 |

### Example 7A: Experimental Data with Ubiquitin for PKC1

To examine if a ubiquitin-tag would improve expression of PKC, a sequence coding for ubiquitin was introduced in front of the PKC1.1 sequence. This sequence is found in construct pCL-SE-0641 which was introduced into SB-0109. Construct pCL-SE-0640 (PKC1.1 without ubiquitin tag) was also introduced into SB-0109 as control. 7-8 separate colonies from each transformation were patched and precultured for 48 h. Assay cultures consisted of YNBD+CAA media with 2.5 mM hexanoic acid. After 24 h, an additional 5 mM hexanoic acid was added to each assay culture. Cultures were quenched after 48 h total growth. Averages and standard deviations were calculated from replicates.

TABLE 11
Effect of ubiquitin tagged PKC1.1 on OA/OL
ratio in SB-0109 (Products in μM).

| | OA | OL | |
|---|---|---|---|
| Strain | (μM) | (μM) | OA/OL |
| SB-0109 + pCL-0640 | 267 ± 3 | 229 ± 29 | 1.2 + 0.1 |
| SB-0109 + pCL-0641 | 268 ± 29 | 155 ± 62 | 1.9 ± 0.6 |

This data shows that the ubiquitin-tagged version of PKC1.1 resulted in a reduction of OL production and significant improvement of the OA/OL ratio.

### Example 7b: In Vivo Activity of PKC4.8 vs “Zipped” Fusion PKC4.8_Zip_1N2D

Plasmids pCL-SE-0676 and -0677 were linearized with AsiSI and transformed into SB-0109-10. Multiple clones per transformation were pre-cultured for 48 hours in 0.5 mL of YNBD (2%)+0.5% casamino acids+100 mM MES pH 6.5. 2 μL of the pre-culture was used to inoculate 0.5 mL of the same media with the addition of 2.5 mM hexanoic acid. At 24 hours, 25 μL of 40% glucose and 100 mM hexanoic acid stock was fed to the cultures for a final concentration of 2% glucose and 5 mM hexanoic acid. At 48 hours, the cultures were quenched and evaluated for olivetolic acid (OA) and olivetol (OL). These data are presented in the table below.

TABLE 12
OA and OL formation from hexanoic acid feeding using
different PKC4.8 and “zipped” version. Products in μM
accumulated in the in vivo assay.

| | OA | OL | |
|---|---|---|---|
| Strain | (μM) | (μM) | OA/OL |
| SB-0109-10 + pCL-SE-0676 | 105 ± 13 | 509 ± 35 | 0.2 |
| SB-0109-10 + pCL-SE-0677 | 179 ± 26 | 262 ± 53 | 0.7 |

These data clearly show that adding dimerization sequences at the N- and C-terminus of PKC4.8 increased both the OA titer and the OA/OL ratio. This is most likely due to the increased expression and/or stability of the “zipped” construct.

### Example 7c: In Vivo Activity of N- and C-Terminal Modifications of PKC4.8 and PKC4.33

As described in detail below, plasmids pCL-SE-0802, pCL-SE-0802.PKS1.1.PKC4.8, pCL-SE-0802.PKS1.1.PKC4.30, pCL-SE-0802.PKS1.1.PKC4.31, pCL-SE-0802.PKS1.1.PKC4.19, pCL-SE-0802.PKS1.1.PKC4.32, pCL-SE-0802.PKS1.1.PKC4.33, pCL-SE-0802.PKS1.1.PKC4.35, pCL-SE-0802.PKS1.1.PKC4.36, pCL-SE-0802.PKS1.1.PKC4.37, pCL-SE-0802.PKS1.1.PKC4.38, pCL-SE-0802.PKS1.1, were transformed into strain SB-0109, which expresses PKS1 and HCS2, and four separate colonies from each transformation were patched and precultured for 48 h. Assay cultures consisted of YPD media with 100 mM MES pH 6.5, 1 mg/mL hygromycin, and 2.5 mM hexanoic acid. After 24 h, an additional 5 mM hexanoic acid and 2% glucose was added to each assay culture. Cultures were quenched with equal volume of ethanol after 48 h total growth and were analyzed by HPLC-MS. Enzymes produced olivetol and olivetolic acid. Averages and standard deviations were calculated from replicates.

TABLE 13
OA and OL formation from hexanoic acid feeding using N-and C-terminal modifications
of PKC4.8 and PKC4.44. Products in μM accumulated in the in vivo assay.

| | OA | OL | |
|---|---|---|---|
| Strain | (μM) | (μM) | OA/OL |
| SB-0109-10 + pCL-SE-0802.PKS1.1.PKC4.8 | 754 ± 21 | 631 ± 24 | 1.2 |
| SB-0109-10 + pCL-SE-0802.PKS1.1.PKC4.30 | 827 ± 16 | 441 ± 13 | 1.9 |
| SB-0109-10 + pCL-SE-0802.PKS1.1.PKC4.31 | 536 ± 11 | 643 ± 13 | 0.8 |
| SB-0109-10 + pCL-SE-0802.PKS1.1.PKC4.19 | 549 ± 43 | 428 ± 41 | 1.3 |
| SB-0109-10 + pCL-SE-0802.PKS1.1.PKC4.32 | 812 ± 17 | 511 ± 12 | 1.6 |
| SB-0109-10 + pCL-SE-0802.PKS1.1.PKC4.33 | 907 ± 36 | 420 ± 27 | 2.2 |
| SB-0109-10 + pCL-SE-0802.PKS1.1.PKC4.35 | 836 ± 63 | 342 ± 30 | 2.4 |
| SB-0109-10 + pCL-SE-0802.PKS1.1.PKC4.36 | 845 ± 76 | 405 ± 36 | 2.1 |
| SB-0109-10 + pCL-SE-0802.PKS1.1.PKC4.37 | 788 ± 19 | 342 ± 8 | 2.3 |
| SB-0109-10 + pCL-SE-0802.PKS1.1.PKC4.38 | 1046 ± 50 | 437 ± 24 | 2.4 |
| SB-0109-10 + pCL-SE-0802.PKS1.1 | 28 ± 0 | 1423 ± 34 | 0.0 |
| SB-0109-10 + pCL-SE-0802 | 15 ± 1 | 701 ± 31 | 0.0 |

These data show that altering the N- and/or C-terminus by truncation or by adding dimerization domains can increase the OA titer and/or OA/OL ratio.

### Example 8: Engineering of PKC4.8 for Improved OA Activity (or OA Analogs Shown in FIG. 2)

Improving the activity of PKC4 and PKC4.8 will eliminate HTAL and PDAL by-products that are forming from inadequate PKC activity and tetraketide accumulation (FIG. 2), and will improve OA titers and OA/OL ratio. Engineering can also direct each PKC's activity towards a tetraketide with specific fatty acid chain (N=2-16, FIG. 2).

In a first step of improving the PKC4/4.8 activity was the creation of a crystal structure model for each enzyme as described in the general methods. The active site was then identified and the olivetolic acid product was docked. All amino acids around the active site that may play a role in the substrate binding, activity and selectivity are identified and will be targeted for mutagenesis. In addition, amino acids in the dimer interface will also be targeted for mutagenesis in order to improve the dimer formation affinity, which may translate in better stability for the complex and may increase expression and tolerance to mutagenesis. Enzymes with good activity and selectivity for OA formation will be selected first using PKC4.8 as template. In subsequent screenings, mutants of PKC4.8 or PKC4 with improved activity cyclizing tetraketides with shorter or longer chain as shown in FIG. 2 will also be identified.

Mutagenesis strategy will involve two parallel approaches. In the first approach, saturation mutagenesis will be performed in all amino acids in the active site. The best mutant from this screen will be selected as a template and additional saturation mutagenesis will be performed in the remaining amino acids (or a subset depending on results of the first screen). This process will be repeated multiple times until satisfactory activity is observed. The second approach will involve parallel mutagenesis of 2-5 amino acids but instead of changing each one with all 20 amino acids, only a subset will be used based on natural variation from homologs and/or amino acids identified in the previous SSM screen. Mutants will be screened as described in Example 5 in in vivo assays expressed in appropriate Yarrowia strains.

TABLE 14
Amino acids targeted for mutagenesis in PKC4 and PKC4.8

| Location in | | |
|---|---|---|
| Enzyme | PKC4 | PKC4.8 |
| Active site | V9, H11, F13, L15, M17, | V9, H11, 113, L15, F17, |
| | M29, N30, Y33, H64, F66, | F29, F30, Y33, H64, V66, |
| | S68, F70, M73, I76, Y79, | S68, F70, V73, 176, Y79, |
| | I80, L86, L88, R89, Y92, | I80, V86, F88, G89, Y92, |
| | F93, L96, F99, V101, | R93, W96, L99, I101, |
| | D103, K105 | D103, T105 |
| Loop | N54-Y62 | N54-Y62 |
| Dimer Interface | V12, I14, A45, Q47, L51, | V12, I14, A45, Q47, L51, |
| | E52, I65, E67, I69, L100, | E52, I65, E67, I69, L100, |
| | F102 | F102 |

As described in detail below, plasmids expressing variants of PKC4.8 were transformed into strain SB-0109, which expresses PKS1 and HCS2, and colonies from each transformation were precultured for 48 h. Assay cultures consisted of YPD media with 100 mM MES pH 6.5, 1 mg/mL hygromycin, and 2.5 mM hexanoic acid. After 24 h, an additional 5 mM hexanoic acid and 2% glucose was added to each assay culture. Cultures were quenched with equal volume of ethanol after 48 h total growth and were analyzed by HPLC-MS. Enzymes produced olivetol and olivetolic acid. For the controls, averages and standard deviations were calculated from replicates.

TABLE 15
OA and OL formation from hexanoic acid feeding using variants
of PKC4.8. Products in μM accumulated in the in vivo assay.

| | OA | OL | |
|---|---|---|---|
| Strain | (μM) | (μM) | OA/OL |
| SB-0109-10 + pCL-SE-0331.PKC4.8 | 173 ± 16 | 253 ± 21 | 0.7 |
| SB-0109-10 + pCL-SE-0331.PKC4.33 | 251 | 221 | 1.1 |
| SB-0109-10 + pCL-SE-0331 | 5 ± 2 | 303 ± 159 | 0.0 |

To verify PKC4.33 activity, plasmids pCL-SE-0331.PKC4.8, pCL-SE-0331.PKC4.33, and pCL-SE-0331 were transformed into strain SB-0109, which expresses PKS1 and HCS2, and four colonies from each transformation were precultured for 48 h. Assay cultures consisted of YPD media with 100 mM MES pH 6.5, 1 mg/mL hygromycin, and 2.5 mM hexanoic acid. After 24 h, an additional 5 mM hexanoic acid and 2% glucose was added to each assay culture. Cultures were quenched with equal volume of ethanol after 48 h total growth and were analyzed by HPLC-MS. Enzymes produced olivetol and olivetolic acid. Averages and standard deviations were calculated from replicates.

### Example 9: Screening of Mutant PKCs

Screening of further mutant and recombined enzymes are described below. The genes were cloned into the plasmid pCL-SE-0802.PKS1.1 and used to transform SB-0741 strain, which expresses HCS2. Six colonies from each transformation were used to inoculate pre-cultures for 48 hours in YDCM media containing 1 mg/mL hygromycin. Assay cultures were performed in the same media with 3 mM hexanoic or butanoic acid added 24 and 30 hours post inoculation. Cultures were quenched with equal volume of ethanol after 48 h total growth and were analyzed by HPLC-MS. Enzymes produced olivetol and olivetolic acid from hexanoic acid and divarinol and divarinic acid from butanoic acid. Averages and standard deviations were calculated from replicates.

Results for the screening are shown in Table 16 and 17

TABLE 16
screening of PKCs in strains grown with hexanoic acid.
Products in μM accumulated in the in vivo assay

| | OA | OL | |
|---|---|---|---|
| Strain | (μM) | (μM) | OA/OL |
| SB-0741+ pCL-SE-0802.PKS1.1.PKC1 | 571 ± 35 | 104 ± 6 | 5.5 |
| SB-0741+ pCL-SE-0802.PKS1.1.PKC4.11 | 567 ± 43 | 145 ± 21 | 3.9 |
| SB-0741+ pCL-SE-0802.PKS1.1.PCK4.15 | 569 ± 33 | 143 ± 18 | 4.0 |

TABLE 17
screening of PKCs in strains grown with butanoic acid.
Products in μM accumulated in the in vivo assay

| | DVA | DVO | DVA/ |
|---|---|---|---|
| Strain | (μM) | (μM) | DVO |
| SB-0741+ pCL-SE-0802.PKS1.1.PKC1 | 787 ± 17 | 195 ± 2 | 4.0 |
| SB-0741+ pCL-SE-0802.PKS1.1.PKC4.11 | 686 ± 66 | 257 ± 30 | 2.7 |
| SB-0741+ pCL-SE-0802.PKS1.1.PKC4.15 | 711 ± 15 | 258 ± 4 | 2.8 |

### Expression of CHIL Proteins for Stabilization of the PKS/PKC and Elimination of Byproducts

The formation of OL, HTAL and PDAL during the synthesis of OA by PKS and PKC may be due to a number of reasons. One likely explanation for HTAL and OL accumulation is that these compounds are formed when tetraketide accumulates due to inadequate PKC cyclization activity (FIG. 2). It is also possible that the formation of these two byproducts may be catalyzed by the PKS as a side reactivity of this enzyme. A similar side reactivity has been shown in another plant Type III polyketide synthase, chalcone synthase (CHS), that catalyzes the formation of HTAL-related derailment byproducts while making its main product, tetrahydroxychalcone (THC). The enzyme's specificity towards THC was greatly improved when chalcone isomerase-like proteins (CHIL) were co-expressed (Waki T, et al Nature Communications 2020, 11, 870). CHIL are non-catalytic proteins that are ubiquitous in plant genomes and in this case are thought to interact with CHS to increase its activity and selectivity (Waki T, et al Nature Communications 2020, 11, 870).

It is possible that PKS (and PKC) may require similar proteins for optimal activity and elimination of byproducts. We therefore identified and cloned putative CHIL proteins, that will be tested with our PKSs for improved activity and specificity.

### Example 10: Testing of Acyl-CoA Synthetase Ability to Increase Acyl-CoA and Improve Final Titers of OA/CBGA and DVA/CBGVA

### Key Findings

Expression of HCSs improved the OA and DVA titers in cells grown in the presence of hexanoic and butyric acid respectively.

Expression of HCS improved OA/CBGA when hexanoic was made in vivo from sugar feed.

The presence of OA titers were also improved when Hexanoyl-CoA was produced through modification of fatty acid biosynthesis/oxidation.

The presence of HCS improved the CBGA & CBGVA titers in cells grown in the presence of hexanoic and butyric acids.

### Example 10a: OA/DVA OR CBGA/CBGVA Formation with Hexanoic/Butyric Acid In Vivo

Plasmids pCL-SE-0539, and -0558 to -0561 were linearized with AsiSI and transformed into SB-0491 as described in the experimental section. Multiple clones per transformation were pre-cultured for 24 h in YNBD containing 0.5% casamino acids and 100 mM MES pH 6.5. 2 μl the preculture was used to inoculate 500 μl of the same medium described above that was supplemented with either 5 mM hexanoic acid or 5 mM butyric acid. After 24 h, the cultures were supplemented with additional glucose (2%) and hexanoic acid (10 mM) or butyric acid (10 mM). After another 24 hours, the cultures were quenched and evaluated for divarinic acid (DVA) and CBGVA production. The results are shown in the tables below:

TABLE 18
DVA and CBGVA formation from butyric acid feeding using
different HCSs. Products in μM accumulated in the in vivo assay.

| | DVA | CBGVA |
|---|---|---|
| Strain | (μM) | (μM) |
| SB-0491 + pCL-SE-0539 | 658 ± 69 | 20 ± 0 |
| SB-0491 + pCL-SE-0558 | 19 ± 6 | 0 ± 0 |
| SB-0491 + pCL-SE-0559 | 12 ± 0 | 0 ± 0 |
| SB-0491 + pCL-SE-0560 | 471 ± 233 | 15 ± 6 |
| SB-0491 + pCL-SE-0561 | 230 ± 83 | 5 ± 3 |

TABLE 19
OA and CBGA formation from Hexanoic acid feeding using
different HCSs. Products in μM accumulated in the in vivo assay.

| | OA | CBGA |
|---|---|---|
| Strain | (μM) | (μM) |
| SB-0491 + pCL-SE-0539 | 205 ± 13 | 84 ± 1 |
| SB-0491 + pCL-SE-0558 | 89 ± 56 | 32 ± 9 |
| SB-0491 + pCL-SE-0559 | 58 ± 28 | 29 ± 15 |
| SB-0491 + pCL-SE-0560 | 170 ± 54 | 54 ± 20 |
| SB-0491 + pCL-SE-0561 | 150 ± 99 | 52 ± 22 |

SB-0491 contains prenyl-transferase activity that enable it to convert divarinic and olivetolic acid to CBGVA and CBGA, respectively. A detailed description of the prenyl transferase that is present in this strain (a fusion of membrane prenyl transferase MPT4 and a mutant geranyl phosphate synthase GPS) is described in a separate filing (Attorney Docket No: CELB-003-WO1, filed on the same day as the present application). However, SB-0491 does not contain polyketide synthase (PKS) and cyclase (PKC) activities that are necessary to produce divarinic and olivetolic acid from butryl-CoA and hexanoyl-CoA, respectively. The PKS and PKC activities are present and identical on the plasmids (pCL-SE-0539, and -0558 to -0561) used in this example. However, the plasmids used in this example contain different HCS sequences. Clearly the amount of final cannabinoid products was related to the acyl-CoA synthetase, with HCS2 having the biggest effect.

The results show that all the HCSs tested enable cells to produce divarinic acid from butyric acid. In addition, strains with HCS2, HCS6, and HCS7 produce enough divarinic acid that some is converted to CBGVA by the prenlytransferase activity in SB-0491. The results also show that all the HCSs tested enable the cells to produce olivetolic acid at sufficient levels that some is converted to CBGA by the prenyl-transferase activity in SB-0491. Another important result to highlight from this example is that the engineered cells described produce CBGA and CBGVA when grown in a medium that is supplemented with hexanoic or butyric acid, respectively, which is a commercially viable approach to producing cannabinoids at scale.

### Example 11: Fusion of HCS with PKS

The flux towards OA, DVA and their analogs can be further increased by fusing the HCS and PKS. There are multiple examples of protein fusions that increase the flux and titers of a product compared to expressing the same proteins separately. Similar to the work described herein, the effect of linker sequences between an acyl-CoA synthetase (coumaroyl-CoA ligase) fused to a stilbene synthase in the final product titer was evaluated (Guo, H et al Mol. Biosyst. 2017, 13, 598-606). The authors showed that linker length and consistency was important and certain linkers improved substantially the kinetic properties of each enzyme in the fusion proteins. Similarly, acyl-CoA synthetases (e.g. the enzymes described herein) will be fused with the PKSs described herein (and their improved mutants that will be identified). Some examples of linker sequences that will be tested are disclosed (SEQ ID NOs 60-62) and some of the fusions that will be tested are also described (SEQ ID NOs 63-65). The fusion proteins will be evaluated for OA (or other cannabinoid formation) when expressed in a cell as described in earlier Examples.

### General and Experimental Methods and Techniques

### Discovery of Natural Sequences

The approach taken to identify new enzymes for each step relied on three general methods. The first involved identifying sequence homologs to known enzymes with the desired activity. The second method relied on literature searches for enzymes that perform similar reactions using the same substrates or enzymes that perform the same reaction with similar substrates. The third method utilized artificial intelligence algorithms to identify potential enzymes based on predicted activities. These methods identified many candidate sequences that were then manually curated and the selected sequences were cloned and characterized.

### Cloning Methods, Vectors and Strains

E. coli Expression Plasmids

Genes for each enzyme were optimized for expression in E. coli, synthesized (Codex DNA), and cloned into the pM264-c vector (ATUM). Genes were sequenced verified and then subcloned into the pD441-NHT expression vector (ATUM) with an N-terminal His tag and TEV protease cleavage site under control of the T5 promoter. Plasmids were transformed into chemically competent E. coli BL21(DE3) cells (NEB), plated on LB agar plates with 50 μg/mL kanamycin, and grown overnight at 37° C. Colony PCR was used to verify gene fragment insertion and positive colonies were inoculated into liquid LB media with 50 μg/mL kanamycin and grown overnight at 37° C. and then diluted with glycerol to create stocks containing 25% glycerol which were stored at −80° C.

### Yarrowia Expression Plasmids

Genes for each enzyme were optimized for expression in Yarrowia, synthesized (Codex DNA), and cloned into the pM264-c vector (ATUM). Genes were sequenced verified and then subcloned into the SapI sites of pCL-SE-0331, pCL-SE-0332, or pCL-SE-0337. Plasmids were transformed into chemically competent E. coli NEB 10-beta cells (NEB), plated on LB agar plates with 50 μg/mL kanamycin or 100 μg/ml carbenicillin, and grown overnight at 37° C. Colony PCR was used to verify gene fragment insertion and positive colonies were inoculated into liquid LB media with the appropriate antibiotic. Cultures were grown overnight at 33° C. and then used for isolating plasmid DNA (Qiagen).

TABLE 21
plasmids with genetic elements:

| | | Key gene(s) |
|---|---|---|
| Plasmid | Host | expressed |
| pM264-c | E. coli | — |
| pCL-SE-0016 | Yarrowia | — |
| pCL-SE-0331 | Yarrowia | — |
| pCL-SE-0331.PKC1 | Yarrowia | PKC1 |
| pCL-SE-0331.PKC4 | Yarrowia | PKC4 |
| pCL-SE-0331.PKC4.8 | Yarrowia | PKC4.8 |
| pCL-SE-0331.PKC4.33 | Yarrowia | PKC4.33 |
| pCL-SE-0332 | Yarrowia | HCS2 |
| pCL-SE-0332.PKS1 | Yarrowia | HCS2, PKS1 |
| pCL-SE-0332.PKS23 | Yarrowia | HCS2, PKS23 |
| pCL-SE-0332.PKS34 | Yarrowia | HCS2, PKS34 |
| pCL-SE-0332.PKS35 | Yarrowia | HCS2, PKS35 |
| pCL-SE-0332.PKS37 | Yarrowia | HCS2, PKS37 |
| pCL-SE-0332.PKS40 | Yarrowia | HCS2, PKS40 |
| pCL-SE-0332.PKS41 | Yarrowia | HCS2, PKS41 |
| pCL-SE-0337 | Yarrowia | — |
| pCL-SE-0345 | Yarrowia | HCS2 |
| pCL-SE-0346 | Yarrowia | HCS3 |
| pCL-SE-0476 | Yarrowia | PKC1.1-F3-PKS1 |
| pCL-SE-0477 | Yarrowia | PKC1.1-F2-PKS1 |
| pCL-SE-0478 | Yarrowia | PKS1-F2-PKC1.1 |
| pCL-SE-0479 | Yarrowia | PKS1-F1-PKC1.1 |
| pCL-SE-0480 | Yarrowia | PKS1-F5-PKC1.1 |
| pCL-SE-0481 | Yarrowia | PKS1-F4-PKC1.1 |
| pCL-SE-0539 | Yarrowia | HCS2, PKS1, PKC1.1 |
| pCL-SE-0558 | Yarrowia | HCS4, PKS1, PKC1.1 |
| pCL-SE-0559 | Yarrowia | HCS5, PKS1, PKC1.1 |
| pCL-SE-0560 | Yarrowia | HCS6, PKS1, PKC1.1 |
| pCL-SE-0561 | Yarrowia | HCS7, PKS1, PKC1.1 |
| pCL-SE-0640 | Yarrowia | PKC1.1 |
| pCL-SE-0641 | Yarrowia | PKC1.1_Ub_1 |
| pCL-SE-0676 | Yarrowia | PKC4.8 |
| pCL-SE-0677 | Yarrowia | PKC4.8_Zip_1N1D |
| pCL-SE-0802 | Yarrowia | — |
| pCL-SE-0802.PKS1.1 | Yarrowia | PKS1.1 |
| pCL-SE-0802.PKS1.1.PKC.PKC1 | Yarrowia | PKS1.1, PKC1 |
| pCL-SE-0802.PKS1.1.PKC.PKC4.8 | Yarrowia | PKS1.1, PKC4.8 |
| pCL-SE-0802.PKS1.1.PKC.PKC4.11 | Yarrowia | PKS1.1, PKC4.11 |
| pCL-SE-0802.PKS1.1.PKC.PKC4.15 | Yarrowia | PKS1.1, PKC4.15 |
| pCL-SE-0802.PKS1.1.PKC.PKC4.19 | Yarrowia | PKS1.1, PKC4.19 |
| pCL-SE-0802.PKS1.1.PKC.PKC4.30 | Yarrowia | PKS1.1, PKC4.30 |
| pCL-SE-0802.PKS1.1.PKC.PKC4.31 | Yarrowia | PKS1.1, PKC4.31 |
| pCL-SE-0802.PKS1.1.PKC.PKC4.32 | Yarrowia | PKS1.1, PKC4.32 |
| pCL-SE-0802.PKS1.1.PKC.PKC4.33 | Yarrowia | PKS1.1, PKC4.33 |
| pCL-SE-0802.PKS1.1.PKC.PKC4.35 | Yarrowia | PKS1.1, PKC4.35 |
| pCL-SE-0802.PKS1.1.PKC.PKC4.36 | Yarrowia | PKS1.1, PKC4.36 |
| pCL-SE-0802.PKS1.1.PKC.PKC4.37 | Yarrowia | PKS1.1, PKC4.37 |
| pCL-SE-0802.PKS1.1.PKC.PKC4.38 | Yarrowia | PKS1.1, PKC4.38 |

### Yarrowia Strains

| Strain | Key genes expressed |
|---|---|
| sCL-SE-0041 | Parent Yarrowia strain |
| SB-0109 | HCS2, PKS1 |
| SB-0264 | HCS2, PKS1, PKC1.1 |
| SB-0665 | HCS2, PKC1.1 |
| SB-0491* | GPS1.1-F11-MPT4, GPS1.1 |
| SB-0741 | HCS2, GPS1.1 |

The description of strain SB-0491 is provided in a patent application filed concurrently by applicants (Attorney Docket CELB-003-001)

E. coli Expression and Purification

To compare the enzyme's activities more accurately, larger cultures of the best hits and controls from the first screen were grown and the enzymes were purified according to the following protocol(s):

Glycerol stocks of each recombinant strain were used to inoculate 2 mL of LB with 50 μg/mL kanamycin. After overnight growth at 37° C., 0.1-0.5 mL were used to inoculate 100-500 mL of TB media (supplemented with [50 μg/mL kanamycin final concentration in the culture]). The cultures were then grown at 37° C. with 250 rpm shaking until an OD600 of approximately 0.8-1.2. At this point, the cultures were transferred to a shaker at room temperature for 30 min (RPM=100-125), after which they were induced with 0.25 mM IPTG. After 16 h at 150 rpm shaking, the cells were pelleted by centrifugation. Cell pellets were frozen at −80 C or immediately taken into the purification procedure(s) described below.

On the date of purification, the cell pellets were thawed and/or immediately resuspended in 10-20 mL of lysis buffer [B-PER© supplemented with 5 mM MgCl2, 100 μg/mL lysozyme, and 2 μL/mL DNaseI (TURBO DNase ThermoFisher)]. After incubation at room temperature or on ice (enzyme contingent) for ˜10 min shaking at low speed (100 rpm), the cell debris were removed by centrifugation at 4750 rpm for 20 min in a pre-chilled rotor at 4° C. Lysates were loaded in pre-equilibrated cobalt spin columns (ThermoFisher, TALON HisPur Cobalt spin column, with 1 or 5 mL resin depending on the initial culture size [100 mL vs 500 mL, respectively]) and 6×His-tagged proteins that were recombinantly overexpressed in E. coli were purified according to manufacturer's protocol, with only minor adjustments that were contingent on the enzyme(s) of interest being purified. The protein(s) of interest were eluted in 50-100 mM HEPES/TRIS pH=7.8/8.0 with 5 mM MgCl2, 50-150 mM NaCl, and 300 mM imidazole. The elution fractions were pooled, and the buffer was exchanged during the concentration steps. The final storage buffer used was, in some cases, enzyme contingent and adjusted accordingly. In most cases, the final buffer the purified enzymes were exchanged into was 50 mM HEPES/TRIS pH=7.5-8.0 with 25-100 mM NaCl/KCl, 5 mM MgCl2, and 10% v/v glycerol. An appropriately sized AMICON Ultra-15 centrifugal unit (new) was used for each enzyme and each enzyme prep. The size for the filter used for this buffer exchange & concentration process always had a MWCO that was three times smaller than the protein of interest (e.g., so the protein would concentrate and not pass through the filter). The centrifugal units were spun at 3500 rpm for 20 mins at 4° C. (3-4 times, refilling with the final dialysis buffer for a complete exchange of buffers [dialysis=final storage buffer). The final spin resulted in anywhere from 0.5-1 mL of dialyzed, purified protein that was carefully titrated out and immediately quantified while on kept on ice. Proteins were quantified using a SpectraMax M2E using the Abs@280 nm (buffer background subtraction & replicate measurements to account for any error via dilution or pipette; N>=6 in almost all cases where total yield was not limiting). The Abs@280 nm was used in combination with the theoretical extinction coefficient of the protein of interest @ 280 nm (https://web.expasy.org/protparam/protparam-doc.html), which has ˜3% error compared to other methods of protein quantification and has fewer pitfalls with regards to interference from any buffer components (e.g., Bradford assay). Proteins were then aliquoted into several 1.5 mL microcentrifuge tubes 50-100 μL in each and then immediately flash frozen in liquid nitrogen to avoid denaturation upon thawing for later use (i.e., slow freezing proteins results in a salt concentration gradient build up, which can be problematic for enzymes prone to aggregation during the thawing process when the enzyme is to be assayed). Enzymes were stored at −80° C. immediately after they were frozen in the labeled microcentrifuge tubes at the volumes. Yields for the enzyme(s) purified in this way sometimes varied; however, in most cases the total yields of purified protein were quite often more than sufficient for the number of assays that they were utilized in (e.g., 500 mL cultures with recombinant expression in E. coli yielded >=5-10 mg of total purified protein, which is approximately 2-5% of the total protein in the cells given a density of 0.8-1.2 (OD600) at the time of induction). A subset of purified enzyme, cell lysate, and the cell pellet from the above steps were always saved and run on an SDS-PAGE gel to assess purity (normalizing load concentrations beforehand). Thus, purity was always ensured (>=˜98%) before assaying any given enzyme; thereby, always obeying Efraim Racker's tenet of protein biochemistry, “Don't waste clean thinking on dirty enzymes.”.

In many cases the AKTA PURE 150 FPLC equipped with a fraction collector & a HisTalon Crude Cobalt Column (5 mL) was utilized to purify proteins. The protocol followed was nearly identical to the above, with only one exception regarding the elution volumes (1 mL into a 96 well plate during elution), which were pooled after the fact by demarcated fractions on the instrument software—that indicated which wells contained the protein of interest based on those wells where protein was titrated by the instrument during elution (e.g., rapid spike in the Abs@280 nm). Proteins were dialyzed & concentrated and then quantified and flash frozen in liquid nitrogen as described above. All enzyme aliquots were stored at −80 C after flash freezing in liquid nitrogen. Purity was ensured prior to any activity assays with the purified protein as described above (e.g., via SDS-PAGE). Noteworthy: All solutions were kept cold on ice or at 4° C. during the purification procedures that were described above.

### Screening in Yarrowia

Overnight YPD (10 g/L yeast extract, 20 g/L peptone, 2% dextrose) cultures were inoculated from glycerol stocks of the appropriate strain and grown at 30° C. with 250 rpm shaking. Once cultures had reached an OD600 of 4-6, cultures were centrifuged at 500×g for 5 min, supernatants were discarded, and cell pellets were resuspended in equal volume of water. Resuspended cells were centrifuged at 500×g for 5 min, supernatants were discarded, and cells were resuspended in a volume (75 μL×OD×Vculture) of transformation cocktail (45% PEG-400, 0.1 M LiAc, 0.1 M DTT, and 25 ug/100 μL SS Salmon Sperm DNA). For each transformation, >1 μg of plasmid DNA was added to 55 μL cells/transformation cocktail and vortexed for 2 s. Transformations were incubated at 39° C. for 1 h with 250 rpm shaking. Transformations were resuspended in 750 μL YPD with 1 M sorbitol and recovered at 30° C. overnight with 250 rpm shaking. The next day, transformations were centrifuged at 500×g for 5 min, supernatants were discarded, and cell pellets were resuspended in 750 YPD. Resuspended transformations were plated on YPD with appropriate selection or YNBD (6.71 g/L yeast nitrogen base+nitrogen, 0.5% casamino acids, 2% dextrose) agar plates and grown at 30° C. for 2 days. Individual colonies were patched onto YPD plates with appropriate selection or YNBD plates and grown at 30° C. overnight. Patches were used to inoculate 0.5 mL YPD with appropriate selection or YNBD precultures in 96w blocks and grown at 30° C. for 24-48 h with 1000 rpm shaking. For assays, 0.5 mL YPD with appropriate selection or YNBD cultures containing substrate were inoculated with 2 μL from precultures and grown at 30° C. for 2-4 days with 1000 rpm shaking with 2% glucose added every 24 h. Assay cultures were quenched by addition of 0.5 mL ethanol with 0.2% formic acid and 0.5 mg/mL pentyl-benzoic acid. Precipitates were pelleted by centrifuging at 4600×g for 10 min and then 200 μL was transferred to fresh plates, sealed, and analyzed via HPLC.

### Analytical Methods

All samples were quenched with equal volume of EtOH containing 0.2 mg/mL internal standard (3,5-Diisopropyl-2-hydroxybenzoic acid CAS #2215-21-6) centrifuged, and clarified solutions were analyzed by HPLC-MS

### Method A

Column: 2.1×50 mm COSMOCORE PBr (Nacalai USA, Inc.)

Mobile Phase: A; 0.1% formic acid in water, B; 0.1% formic acid in acetonitrile

Flow: 0.45 mL/min

Temp: %50 Celsius

Gradient: 20% B at 0 min, 70% B at 2.3 min, 89% B at 4.2 min, 20% B at 4.3 min, 20% B at 6 min

Detection: UV DAD and QToF MS

All compounds except PDAL-C6, were confirmed and quantified based on authentic standards. For PDAL-C6 and PDAL-C4 (if present) the quantification was made from authentic PDAL-C2 (4-hydroxy-6-methyl-2-pyrone, CAS #675-10-5)

| | Retention |
|---|---|
| Compound | Time |
| PDAL C2 | 0.49 |
| Butyric acid | 0.60 |
| OL C2 | 0.72 |
| PDAL C4 | 1.14 |
| OA C2 | 1.25 |
| Hexanoic acid | 1.53 |
| OL C4 (Divarinol) | 1.65 |
| OA C4 (Divarinic acid) | 1.89 |
| PDAL C6 | 2.05 |
| OL C6 (Olivetol) | 2.32 |
| OA C6 (Olivetolic acid) | 2.43 |
| Internal Std. | 3.10 |

### Method B

Column: 2.1×50 mm COSMOCORE PBr (Nacalai USA, Inc.)

Mobile Phase: A; 0.1% formic acid in water, B; 0.1% formic acid in acetonitrile

Flow: 0.45 mL/min

Temp: %50 Celsius

Gradient: 20% B at 0 min, 35% B at 1 min, 40% B at 2.5 min, 70% B at 3 min, 90% B at 5 min, 20% B at 5.5 min, 20% B at 8 min

Detection: UV DAD and QToF MS

All olivetol derivatives were identified a) from their UV spectra that was identical to olivetol and divarinol and b) from their MS analysis confirming the molecular mass. The quantification was based on UV at either 210 nm or 275 nm using OL absorbance coefficient for these wavelengths.

| | Retention |
|---|---|
| Compound | Time |
| OL C2 | 0.49 |
| OL C4 | 1.05 |
| OL C6 | 1.84 |
| OL C8 | 3.22 |
| OL C10 | N.D. |
| OL C12 | 4.44 |
| OL C14 | 5.34 |
| OL C16 | 6.47 |
| OL C16:1 | 5.77 |
| OL C18:1 | 6.96 |
| OL C18:2 | 6.32 |
| Internal Std. | 3.63 |

### Modeling and Mutagenesis

As described in all engineering projects in this work, prior to any mutagenesis approach structural models of the proteins were created. For this, a variety of commercial and free software packages are available that were used to make structure models using crystal structures of homologous proteins as templates. The selection of the template structures used in the homology modelling process considered three important factors: i) sequence identity between the template enzyme(s) and the target enzyme(s) [only those with >30% sequence identity were used]; ii) the atomic resolution at which the template enzyme(s) were solved; and iii) The percent of sequence coverage between the target enzyme and the template enzyme(s) (i.e., differences in the length of the enzymes). Using this approach 8 to 10 templates were used to generate the homology models. The homology models were evaluated for accuracy using specific software (MolProbity) and if necessary, further refinement and correction of the structure models was achieved using secondary software. Refinement of models entailed rotamer optimization and then the use of GROMACS and energy minimization. Specifically, the top model from multi-template-based modelling was placed in a cubic box with edges 2 nm from any part of the protein being modelled. Periodic boundary conditions were defined, the system was solvated (TIP5P water model; current updated version; gold standard for MD), and the charge of the system neutralized with Na2+ or Cl2− contingent on the protein and overall charge of the system. Models were then refined using the amber99sb-ildn force-field (widely used force-field for MD), and the simulation was conducted until the potential energy of the entire system converged. The energy minimized PDB was extracted without the neutralizing ions and explicit water molecules, and then subjected to quality improvement using MolProbity. In all cases, refinement improved the overall quality of the initial model significantly.

Finally, the appropriate substrates were docked in the active site using a AutoDock Vina software package and iterative changes to the grid search size. The top two (of a number of possible orientations) docking poses for substrates were selected based on calculated binding energy and the orientation in the active site that brings substrates at the right position for reaction. After this modeling exercise was completed, amino acids in the active site that are 5 Å from each substrate were identified and were selected for mutagenesis.

Amino Acid Sequences
PKS1
(SEQ ID NO: 1)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSST
VLFVMDELRKRSLEEGKSTTGDGFEWGVLFGFGPGLTVERVVVRSVPIKY
PKS23
(SEQ ID NO: 2)
MGSAPPAATVQEMRRAQRADGPAAVLAIGTANPPSIMPQDDYPDYY
FRVTNSEHLTDLKAKLSRICNHNKSGIRQRYLHLNEELLAANPGFIDPKRPSLDERVE
MASAAVPELAAKAAAKAIAEWGRPATDITHLIFSTYSGARAPSGDRRLASLLGLRPT
VSRTILSLHGCYGGGRALQLAKELAENNRGARVLVACSELTLIAFYGPEGGCVDNIIG
QTLFGDGAGAVIVGADPVGAPAERPLFEMVFASQTTIPETEDAISMQYSKCGMEYHL
SSRVPRVLGSNVERCLVDTFRTLGVSVAWNDLFWAIHPGGRAILDNIEEVLRLEDGK
LAASRHVLSEFGNMSGTTVIFVLDELRRRRAAAAKQGGQAPEWGVMMAFGPGITVE
TMVLHAPSNLELEGN
PKS34
(SEQ ID NO: 3)
MGSAPATVQEMRRAQRADGPAAVLGIGTANPPTCLAQDDYPDYYFR
VTNSEHLTDLKGKLTRICNKSGIKQRYIHLNEDLLAANPDFTDRTRPSLDARVDIASA
AVPELAAAAAAKAIAEWGRPATDITHLVFSTYSGARAPSADRRLASLLGLRPTVSRTI
LNLHGCYGGGRSLQLAKELAENNRGARVLVACSEITLIAFYGPEGGCADNILGQALF
GDGAGAVIVGADPVAPVERPLYEMAFASQTTIPETEDAISMQINKGGMEYHISNQVP
RLLGCNVERCLVDAFRALGVSAAWNDLFWAIHPGGRAILDHIEGVLGLDDTKLAAS
RHVLSEFGNMSGTTVIFVLDELRRRRAAMAKQGGEAPEWGVMMAFGPGITIETMVL
HAPSNLDLKGN
PKS35
(SEQ ID NO: 4)
MGSAPATAQEMRRAQRADGPAAVLGIGTANPPTCLAQDDYPDYYFR
VTNSEHLTDLKGKLTRICNKSGIKQRYIHLNEDLLAANPDFADRTRPSLDARVDIASA
AVPELAAAAAAKAIAEWGRPATDITHLVFSTYSGARAPSADRRLASLLGLRPTVSRTI
LNLHGCYGGGRSLQLAKELAENNRGARVLVACSEITLIAFYGPEGGCADNILGQALF
GDGAGAVIVGADPVAPVERPLFEMAFASQTTIPETEDAISMQINKGGMEYHISNQVPR
LLGCNVERCLVDAFRALGVSAAWNDLFWAIHPGGRAILDHIEGVLGLDDSKLAASR
HVLSEFGNMSGTTVIFVLDELRRRRAAMAMQGGEAPEWGVMMAFGPGITIETMVLH
APSNLDLKGN
PKS36
(SEQ ID NO: 5)
MGSMGKALPATVDEIRRAQRAEGPAAVLAIGTANPPTIMPQDDYPDY
YFRVTNSEHLTDLKAKLSRICNHNKSGIRQRYLHLNEELLAANPGFIDPKRPSLDERV
EMASAAVPELAAKAATKAIAEWGRPATDITHLIFSTYSGARAPSGDRRLASLLGLRPT
VSRTILNLHGCYGGGRSLQLAKEIAENNRGARVLVACSELTLIAFYGPEGGCVDNIIG
QTLFGDGAGAVVVGADPDAAVERPLFEMAFATQTTIPESEDAISMQYSKCGMEYHL
SSKVPRLIGCNVERSLVDTFRTLGVTAAWNDLFWAVHPGGRAILDNIEEVLGLEDDK
LAASRHVLSEFGNMSGTTVIFVLDELRRRRAAAAKQGGETPEWGVLMAFGPGITIETI
VLHTPSNPELEGN
PKS37
(SEQ ID NO: 6)
MGSAPATIGDMRRAQRADGPAAVLGIGTANPPTCLAQDEYPDYYFR
VTKSEHLTDLKGKLTRICNKSGIKQRFIHLDEQLLAANPDFTDRTLPSLDARVEIASAA
VPELAASAAAKAIADWGRPATDITHLIFSTYSGARAPSADRRLASLLGLSPTVSRTILN
LHGCYGGGRSLQLAKELAENNRGARVLVACSEITLIAFYGPEGGCPDNILGQALFGD
GAGAVIVGADPVSPVERPLFEMAFASQTTIPETEDAISMQINKGGMEYHISNQVPRLL
GCNVERCLVDAFGALGINNNDWNDLFWAIHPGGRAILDHIEGVLGLDDGKLAASRH
VLSEFGNMSGTTVIFVLDELRRRRGLAVKQEEGKAPEWGVMMAFGPGITIETMVLR
APAANLEGN
PKS40
(SEQ ID NO: 7)
MGSAPATVVGEIRRAQRADGPAAVLGIGTANPPTSMAQDEYPDYYF
RVTNSEHLTDLKAKLTRICKKSGIKQRFMHLNEDLLAANPDFTDRTLPSLDARVDIAS
AAVPELAAAAAAKAITEWGRPATEITHLIFSTYSGARAPSADRRLASLLGLSPTVSRT
MLNLHGCYGGGRSLQLAKELAENNRGARVLVACSEITLIAFYGPEGGCPDNILGQSL
FGDGAGAVIIGADPVGPVERPLFEMAFASQTTIPGTEDDISMEINKGGMEYHISNKVP
RLLGCNVERCLIDAFGALGVSAKWNDLFWAIHPGGRAILDHIEGVLGLDDGKLAASR
HVLSEFGNMSGTTVIFVLDELRHRRVAKLDGEAPEWGVMMAFGPGITIETMVLHAP
ASLEGN
PKS41
(SEQ ID NO: 8)
MGSAPAATAGEIRRAQRADGPAAVLAIGTANPPTSMTQDEYPDYYFR
VTNSEHLTDLKAKLTRICKKSGIKQRFMHLNEELLAANPDFTDRTLPSLDARVDIASA
AVPELAAAAAAKAIAEWGRPATEITHLIFSTYSGARAPSADRRLASLLGLSPTVSRTM
LNLHGCYGGGRSLQLAKELAENNRGARVLVACSEITLIAFYGPEGGCPDNILGQALF
GDGAGAVIIGADPVGPVERPLFEMAFASQTTIPGTEDDISMEINKGGMEYHISNKVPR
LLGCNVERCLIDAFGALGVSAKWNDLFWAIHPGGRAILDHIEGVLGLDDGKLAASRH
VLSEFGNMSGTTVIFVLDELRRRRATKQEGVEAPEWGVMMAFGPGITIETMVLHAPA
ILDEN
PKC1
(SEQ ID NO: 9)
MAVKHLIVLKFKDEITEAQKEEFFKTYVNLVNIIPAMKDVYWGKDVT
QKNKEEGYTHIVEVTFESVETIQDYIIHPAHVGFGDVYRSFWEKLLIFDYTPRK
PKC1.1
(SEQ ID NO: 12)
MAVKHLIVLKFKDEITEAQKEEFFKTFVNLVNIIPAMKDVYWGKDVT
QKNKEEGYTHIVEVTFESVETIQDYIIHPAHVGFGDVYRSFWEKLLIFDYTPRK
PKC4
(SEQ ID NO: 10)
MGEANKGVVKHVFILKMKEGLSNDQIEQMNKDYANLVNLVPSMKA
LQWGKLEVNNKLGNGGYTHIFESTFESMEGVAEYADHPAHLHLRNLYFHTLDKFLV
FDYKPTIVLPNSSY
PKC4.8
(SEQ ID NO: 11)
MGEANKGVVKHVIILKFKEGITEAQKEEFFKTYVNLVNLVPAMKAV
QWGKLEVNNKLGNGGYTHIVESTFESVETIQDYIIHPAHVGFGDVYRSFWEKLLIFDY
TPTIVLPNSSY
F1
(SEQ ID NO: 13)
GGGGSGGGGSAEAAAKAEAAAKAGGGGSGGGGS
F2
(SEQ ID NO: 14)
GGAEAAAKEAAAKAGGSGGGSGGGGSGGS
F3
(SEQ ID NO: 15)
GGAEAAAKEAAAKAAEAAAKEAAAKAGGGSPGPGPGGGS
F4
(SEQ ID NO: 16)
GSSSSSSGSSSSSSGSSSSSSGSSSSSSGSSSSSSG
F5
(SEQ ID NO: 17)
GGGGSGGGGSGGGGS
F6
(SEQ ID NO: 18)
GGEAAAKEAAAKEAAAKGG
F7
(SEQ ID NO: 19)
GGAEAAAKEAAAKAPAPAPAG
F8
(SEQ ID NO: 20)
GTPTPTPTPTG
F9
(SEQ ID NO: 21)
GGGGSGGGGSGGGGSGGGGSGGGGSGGGGS
F10
(SEQ ID NO: 22)
GGAEAAAKEAAAKAAEAAAKEAAAKAAEAAAKEAAAKAAEAAAK
EAAAKAGG
F11
(SEQ ID NO: 23)
GGAEAAAKEAAAKAGGSGGGSGGGGSGGSGGGGSGGGGS
F12
(SEQ ID NO: 24)
GGGGSGGGGS
F13
(SEQ ID NO: 25)
GGSGSAGSAAGSGEFGG
F14
(SEQ ID NO: 26)
GGAEAAAKEAAAKAPAPAPAEAAAKEAAAKAGG
F15
(SEQ ID NO: 27)
GGSGGAEAAAKEAAAKAGGSGG
PKC1.1-F3-PKS1
(SEQ ID NO: 34)
MAVKHLIVLKFKDEITEAQKEEFFKTFVNLVNIIPAMKDVYWGKDVT
QKNKEEGYTHIVEVTFESVETIQDYIIHPAHVGFGDVYRSFWEKLLIFDYTPRKGGAE
AAAKEAAAKAAEAAAKEAAAKAGGGSPGPGPGGGSMNHLRAEGPASVLAIGTANP
ENILLQDEFPDYYFRVTKSEHMTQLKEKFRKICDKSMIRKRNCFLNEEHLKQNPRLVE
HEMQTLDARQDMLVVEVPKLGKDACAKAIKEWGQPKSKITHLIFTSASTTDMPGAD
YHCAKLLGLSPSVKRVMMYQLGCYGGGTVLRIAKDIAENNKGARVLAVCCDIMAC
LFRGPSESDLELLVGQAIFGDGAAAVIVGAEPDESVGERPIFELVSTGQTILPNSEGTIG
GHIREAGLIFDLHKDVPMLISNNIEKCLIEAFTPIGISDWNSIFWITHPGGKAILDKVEE
KLHLKSDKFVDSRHVLSEHGNMSSSTVLFVMDELRKRSLEEGKSTTGDGFEWGVLF
GFGPGLTVERVVVRSVPIKY
PKC1.1-F2-PKS1
(SEQ ID NO: 35)
MAVKHLIVLKFKDEITEAQKEEFFKTFVNLVNIIPAMKDVYWGKDVT
QKNKEEGYTHIVEVTFESVETIQDYIIHPAHVGFGDVYRSFWEKLLIFDYTPRKGGAE
AAAKEAAAKAGGSGGGSGGGGSGGSMNHLRAEGPASVLAIGTANPENILLQDEFPD
YYFRVTKSEHMTQLKEKFRKICDKSMIRKRNCFLNEEHLKQNPRLVEHEMQTLDAR
QDMLVVEVPKLGKDACAKAIKEWGQPKSKITHLIFTSASTTDMPGADYHCAKLLGL
SPSVKRVMMYQLGCYGGGTVLRIAKDIAENNKGARVLAVCCDIMACLFRGPSESDL
ELLVGQAIFGDGAAAVIVGAEPDESVGERPIFELVSTGQTILPNSEGTIGGHIREAGLIF
DLHKDVPMLISNNIEKCLIEAFTPIGISDWNSIFWITHPGGKAILDKVEEKLHLKSDKF
VDSRHVLSEHGNMSSSTVLFVMDELRKRSLEEGKSTTGDGFEWGVLFGFGPGLTVER
VVVRSVPIKY
PKS1-F2-PKC1.1
(SEQ ID NO: 36)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSST
VLFVMDELRKRSLEEGKSTTGDGFEWGVLFGFGPGLTVERVVVRSVPIKYGGAEAA
AKEAAAKAGGSGGGSGGGGSGGSMAVKHLIVLKFKDEITEAQKEEFFKTFVNLVNII
PAMKDVYWGKDVTQKNKEEGYTHIVEVTFESVETIQDYIIHPAHVGFGDVYRSFWE
KLLIFDYTPRK
PKS1-F1-PKC1.1
(SEQ ID NO: 37)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSST
VLFVMDELRKRSLEEGKSTTGDGFEWGVLFGFGPGLTVERVVVRSVPIKYGGGGSG
GGGSAEAAAKAEAAAKAGGGGSGGGGSMAVKHLIVLKFKDEITEAQKEEFFKTFVN
LVNIIPAMKDVYWGKDVTQKNKEEGYTHIVEVTFESVETIQDYIIHPAHVGFGDVYR
SFWEKLLIFDYTPRK
PKS1-F5-PKC1.1
(SEQ ID NO: 38)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSST
VLFVMDELRKRSLEEGKSTTGDGFEWGVLFGFGPGLTVERVVVRSVPIKYGGGGSG
GGGSGGGGSMAVKHLIVLKFKDEITEAQKEEFFKTFVNLVNIIPAMKDVYWGKDVT
QKNKEEGYTHIVEVTFESVETIQDYIIHPAHVGFGDVYRSFWEKLLIFDYTPRK
PKS1-F4-PKC1.1
(SEQ ID NO: 39)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSST
VLFVMDELRKRSLEEGKSTTGDGFEWGVLFGFGPGLTVERVVVRSVPIKYGSSSSSSG
SSSSSSGSSSSSSGSSSSSSGSSSSSSGMAVKHLIVLKFKDEITEAQKEEFFKTFVNLVNII
PAMKDVYWGKDVTQKNKEEGYTHIVEVTFESVETIQDYIIHPAHVGFGDVYRSFWE
KLLIFDYTPRK
Zip_1N
(SEQ ID NO: 47)
MSPEDENRELEEKIRELKEKNEELKREIKYLEE
Zip_1C
(SEQ ID NO: 48)
GGGGSPEDKNEELKREIERLEEENRELERKIEYLKR
Zip_1D
(SEQ ID 68)
SPEDKNEELKREIERLEEENRELERKIEYLKR
Zip_2C
(SEQ ID NO: 49)
GPEDKNEELKREIERLEEENRELERKIEYLKR
PKC1_Zip_1N1C
(SEQ ID NO: 40)
MSPEDENRELEEKIRELKEKNEELKREIKYLEEAVKHLIVLKFKDEITE
AQKEEFFKTYVNLVNIIPAGGGGSMKDVYWGKDVTQKNKEEGYTHIVEVTFESVETI
QDYIIHPAHVGFGDVYRSFWEKLLIFDYTPRKGGGGSPEDKNEELKREIERLEEENRE
LERKIEYLKR
PKC1_Zip_1N2C
(SEQ ID NO: 41)
MSPEDENRELEEKIRELKEKNEELKREIKYLEEAVKHLIVLKFKDEITE
AQKEEFFKTYVNLVNIIPAGSMKDVYWGKDVTQKNKEEGYTHIVEVTFESVETIQDY
IIHPAHVGFGDVYRSFWEKLLIFDYTPRKGPEDKNEELKREIERLEEENRELERKIEYL
KR
PKC4.8_Zip_1N2D
(SEQ ID NO: 42)
MSPEDENRELEEKIRELKEKNEELKREIKYLEEGVVKHVIILKFKEGIT
EAQKEEFFKTYVNLVNLVPAMKAVQWGKLEVNNKLGNGGYTHIVESTFESVETIQD
YIIHPAHVGFGDVYRSFWEKLLIFDYTPTIVLPNSSYSPEDKNEELKREIERLEEENREL
ERKIEYLKR
PKC4_Ub_1
(SEQ ID NO: 58)
MQIFVKTLTGKTITLEVESSDTIDNVKSKIQDKEGIPPDQQRLIFAGKQ
LEDGRTLSDYNIQKESTLHLVLRLRGGGEANKGVVKHVFILKMKEGLSNDQIEQMN
KDYANLVNLVPSMKALQWGKLEVNNKLGNGGYTHIFESTFESMEGVAEYADHPAH
LHLRNLYFHTLDKFLVFDYKPTIVLPNSSY
PKC4.8_Ub_1
(SEQ ID NO: 59)
MQIFVKTLTGKTITLEVESSDTIDNVKSKIQDKEGIPPDQQRLIFAGKQ
LEDGRTLSDYNIQKESTLHLVLRLRGGGEANKGVVKHVIILKFKEGITEAQKEEFFKT
YVNLVNLVPAMKAVQWGKLEVNNKLGNGGYTHIVESTFESVETIQDYIIHPAHVGF
GDVYRSFWEKLLIFDYTPTIVLPNSSY
PKC1.1_Ub_1
(SEQ ID NO: 43)
MQIFVKTLTGKTITLEVESSDTIDNVKSKIQDKEGIPPDQQRLIFAGKQ
LEDGRTLSDYNIQKESTLHLVLRLRGGAVKHLIVLKFKDEITEAQKEEFFKTFVNLVN
IIPAMKDVYWGKDVTQKNKEEGYTHIVEVTFESVETIQDYIIHPAHVGFGDVYRSFW
EKLLIFDYTPRK
P3-PKC1.1
(SEQ ID NO: 44)
MGSSPEDEIQQLEEEIAQLEQKNAALKEKNQALKYGSGGAEAAAKEA
AAKAGGSGGGSGGGGSGGSMAVKHLIVLKFKDEITEAQKEEFFKTFVNLVNIIPAMK
DVYWGKDVTQKNKEEGYTHIVEVTFESVETIQDYIIHPAHVGFGDVYRSFWEKLLIF
DYTPRK
PKC1.1-P4
(SEQ ID NO: 45)
MAVKHLIVLKFKDEITEAQKEEFFKTFVNLVNIIPAMKDVYWGKDVT
QKNKEEGYTHIVEVTFESVETIQDYIIHPAHVGFGDVYRSFWEKLLIFDYTPRKGGAE
AAAKEAAAKAGGSGGGSGGGGSGGSGSPEDKIAQLKQKIQALKQENQQLEEENAAL
EYGGSG
P3-PKC1.1-PKS1
(SEQ ID NO: 46)
MGSSPEDEIQQLEEEIAQLEQKNAALKEKNQALKYGSGGAEAAAKEA
AAKAGGSGGGSGGGGSGGSMAVKHLIVLKFKDEITEAQKEEFFKTFVNLVNIIPAMK
DVYWGKDVTQKNKEEGYTHIVEVTFESVETIQDYIIHPAHVGFGDVYRSFWEKLLIF
DYTPRKGGAEAAAKEAAAKAGGSGGGSGGGGSGGSMNHLRAEGPASVLAIGTANP
ENILLQDEFPDYYFRVTKSEHMTQLKEKFRKICDKSMIRKRNCFLNEEHLKQNPRLVE
HEMQTLDARQDMLVVEVPKLGKDACAKAIKEWGQPKSKITHLIFTSASTTDMPGAD
YHCAKLLGLSPSVKRVMMYQLGCYGGGTVLRIAKDIAENNKGARVLAVCCDIMAC
LFRGPSESDLELLVGQAIFGDGAAAVIVGAEPDESVGERPIFELVSTGQTILPNSEGTIG
GHIREAGLIFDLHKDVPMLISNNIEKCLIEAFTPIGISDWNSIFWITHPGGKAILDKVEE
KLHLKSDKFVDSRHVLSEHGNMSSSTVLFVMDELRKRSLEEGKSTTGDGFEWGVLF
GFGPGLTVERVVVRSVPIKY
HCS2
(SEQ ID NO: 28)
MASEENDLVFPSKEFSGQALVSSPQQYMEMHKRSMDDPAAFWSDIA
SEFYWKQKWGDQVFSENLDVRKGPISIEWFKGGITNICYNCLDKNVEAGLGDKTAIH
WEGNELGVDASLTYSELLQRVCQLANYLKDNGVKKGDAVVIYLPMLMELPIAMLA
CARIGAVHSVVFAGFSADSLAQRIVDCKPNVILTCNAVKRGPKTINLKAIVDAALDQS
SKDGVSVGICLTYDNSLATTRENTKWQNGRDVWWQDVISQYPTSCEVEWVDAEDP
LFLLYTSGSTGKPKGVLHTTGGYMIYTATTFKYAFDYKSTDVYWCTADCGWIGGHS
YVTYGPMLNGATVVVFEGAPNYPDPGRCWDIVDKYKVSIFYTAPTLVRSLMRDDDK
FVTRHSRKSLRVLGSAGEPINPSAWRWFFNVVGDSRCPISDTWGQTETGGFMITPLPG
AWPQKPGSATFPFFGVQPVIVDEKGNEIEGECSGYLCVKGSWPGAFRTLFGDHERYE
TTYFKPFAGYYFSGDGCSRDKDGYYWLTGRVDDVINVSGHRIGTAEVESALVLHPQ
CAEAAVVGIEHEVKGQGIYAFVTLLEGVPYSEELRKSLVLMVRNQIGAFAAPDRIHW
APGLPKTRSGKIMRRILRKIASRQLEELGDTSTLADPSVVDQLIALADV
HCS3
(SEQ ID NO: 29)
MSKDTSVLLEEKRVFIPHYTVVEEAHIKNWEAELEKGKDHENYWAE
KAERFEWFRKWDRVLDESNRPFYRWFVNGKINMTYNAVDRWLDTDKRNQVAILYV
NERGDERKLTYYELYREVNRTANALKSLGIKKGDAVALYLPMCPELVISMLACAKIG
AVHSVTYSGLSVGALVERLNDARAKIIITADGTYRRGGVIKLKPIVDEAILQCPTIETT
VVVKHTDIDIEMSDISGREMLFDKLIEGEGDRCDAEEMDAEDPLFILYTSGSTGKPKG
VLHTTGGYMVGVASTLEMTFDIHNGDLWWCTADIGWITGHSYVVYGPLLLGTTTLL
YEGAPDYPDPGVWWSIVEKYGVTKFYTAPTAIRHLMRFGDKHPKRYNLESLKILGTV
GGPINPEAWMWYYRNIGREKCPIIDTWGQIETGMHLIAPLPVTPLKPGSVTKPLPGIEA
DVVDENGDPVPLGKGGFLVIRKPWPAMFRTLFNDEQRYIDVYWKQIPGGVYTAGD
MARKDEDGYFWIQGRSDDVLNIAGHRIGTAEVESVFVAHPAVAEAAVIGKADPIKGE
VIKAFLILKKGHKLNAALIEELKRHLRHELGPVAVVGEMVQVDSLPKTRSGKIMRRIL
RAKEEGEDLGDTSTLEE
HCS4
(SEQ ID NO: 30)
MWNDHDSPEEFNFASDVLDYWAQMEEEGKRGPSPAFWWVNGQGD
EIKWSFRKLRDLTCRTANVFEQICGLQQGDHLALILPRVPEWWLVTVGCMRTGIIFM
PGTTQLKAKDILYRIQISRAKAIVTTASLVPEVESVASECPDLKTKLVVSDHSHEGWL
DFCSLIKSASPDHTCIKSKMKDPMAIFFTSGTTGYPKMAKHNQGLAFRSYIPSCRKLL
KLKTSDILWCMSDPGWILATVGCLIEPWTSGCTVFIHHLPQFDPKVIVEVLFKYPITQC
LAAPGVYRMVLQQKTSNLRFPTLEHCTTGGESLLPEEYEQWKQRTGLSIHEVYGQSE
TGISSATLREMKIKRGSIGKAILPFDLQIIDEKGNIPPNTEGYIGIRIKPTRPLGLFMEYE
NSPESTSEVECGDFYNSGDRATIDEEGYIWFLGRGDDVINASGYRIGPVEVENALAEH
PAVAESAVVSSPDKDRGEVVKAFIVLNPEFLSHDQEQLIKELQHHVKSVTAPYKYPR
KVEFVSELPKTVTGKIKRKELRNKEFGQL
HCS5
(SEQ ID NO: 31)
MNTKKKFTSLIHLLCYRGTNKPNQKAYTFIGNGKTETASLTYGELEK
RSRAIAAQLQEMGVTRGERALLLYSQPLDFICAFFGCLYAGVIAIPAPPPDAIRLKRTL
PRLQACVKDAQVSLVLTTSQIYSQFPSEWQKDFEYNNMLLWLFTEEISEQLANQWQE
LKINLDAIAYLQYTSGSTSTPKGVIVTHNNVMHHSAYIKQAWNYTSDSIAATWMPYF
HDYGLIDGLIQPIYSGITCYVMSPLTFVRRPTCWLEVISKYKVTHSQSPNFGYDYCVR
QVTSEQLNNLDLRSWKTASNGAEPIRKDTIEKFIKTFEPCGFRATAFFPSYGLAEATLL
VATKSHNDVPEIASIAASALEKNQIVECDGNQKGTRYVVSCGFPICGIKVIIINPNTLTR
CASDEVGEIWVSDLSVAQGYWNRPEETKHTFEAYLADTGEGPFLRTGDLGFIKNGQL
FVTGRLKDVIIIRGQNHYPQDLEFTVEQSHPALRKNSEAAFGIEVDGEEKLVVVQEVE
RSWLRKLDFDQVNGDIRQALMEQHELQVYAIALIKPGSIPKTSSGKIMRHACRIKFLE
GTLEVINSGGGNPEHLRRLTVMSVMRATPKNGAHLG
HCS6
(SEQ ID NO: 32)
MLGQMMRNQLVIGSLVEHAARYHGAREVVSVETSGEVTRSCWKEV
ELRARKLASALGKMGLTPSDRCATIAWNNIRHLEVYYAVSGAGMVCHTINPRLFIEQ
ITYVINHAEDKVVLLDDTFLPIIAEIHGSLPKVKAFVLMAHNNSNASAQMPGLIAYED
LIGQGDDNYIWPDVDENEASSLCYTSGTTGNPKGVLYSHRSTVLHSMTTAMPDTLNL
SARDTILPVVPMFHVNAWGTPYSAAMVGAKLVLPGPALDGASLSKLIASEGVSIALG
VPVVWQGLLAAQAGNGSKSQSLTRVVVGGSACPASMIREFNDIYGVEVIHAWGMTE
LSPFGTANTPLAHHVDLSPDEKLSLRKSQGRPPYGVELKIVNDEGIRLPEDGRSKGNL
MARGHWVIKDYFHSDPGSTLSDGWFSTGDVATIDSDGFMTICDRAKDIIKSGGEWIS
TVELESIAIAHPHIVDAAVIAARHEKWDERPLLIAVKSPNSELTSGEVCNYFADKVAR
WQIPDAAIFVEELPRNGTGKILKNRLREKYGDILLRSSSSVC
HCS7
(SEQ ID NO: 33)
MMVPTLEHELAPNEANHVPLSPLSFLKRAAQVYPQRDAVIYGARRYS
YRQLHERSRALASALERVGVQPGERVAILAPNIPEMLEAHYGVPGAGAVLVCINIRLE
GRSIAFILRHCAAKVLICDREFGAVANQALAMLDAPPLLVGIDDDQAERADLAHDLD
YEAFLAQGDPARPLSAPQNEWQSIAINYTSGTTGDPKGVVLHHRGAYLNACAGALIF
QLGPRSVYLWTLPMFHCNGWSHTWAVTLSGGTHVCLRKVQPDAINAAIAEHAVTH
LSAAPVVMSMLIHAEHASAPPVPVSVITGGAAPPSAVIAAMEARGFNITHAYGMTES
YGPSTLCLWQPGVDELPLEARAQFMSRQGVAHPLLEEATVLDTDTGRPVPADGLTL
GELVVRGNTVMKGYLHNPEATRAALANGWLHTGDLAVLHLDGYVEIKDRAKDIIIS
GGENISSLEIEEVLYQHPEVVEAAVVARPDSRWGETPHAFVTLRADALASGDDLVRW
CRERLAHFKAPRHVSLVDLPKTATGKIQKFVLREWARQQEAQIADAEH
CHIL1
(SEQ ID NO: 50)
MATSDGSSNAATKEEAVQVEPKTGISFPVKLDDGKILYCVGYNKKSL
LGLSIKAYGFGLYVDSDKLKDVLKSKIEKAPSKPTEEMYQLAIDGDFGMTIKMVVSF
SGVKLSMAKKGFTEAMRESMKKLTGQKNEELSNKVFGTTSDKIKLRLGSEMIVSKLP
GYVLETKVNGELVSRVESELLCRAYFRNYLGEDTLECEKESREMFGQSMLSLF
CHIL2
(SEQ ID NO: 51)
MANNMVMVHEIPFPTEIKTTKPLSLLGYGITDMEIHFLQIKFTAIGVYL
DPDVVKHVQQWKGKKGNELAEDDDFFDALISAPVEKYLRIVVIKEIKGSQYGVQLES
SVRDRLAADDKYEEEEEAALEKIVEFFQSKYFKKDTLITFHFPATSPTAEIVVTIEGKE
EFKLDVENENVVEMIKKWYLGGTTGASPSTISSLADNLSAQLSK
CHIL3
(SEQ ID NO: 52)
MATALNSKNASSNTAVHIEPKTGIAFPVKLDDGKSLNSVGLRKKSLL
GMGIKVFGFGLYADNEKLKNLLKLKIGKSPAKPTEEMYQLVIDGDIGLTHKIVIAYSG
LKMNMFKKAFSEALGESIMKLNGGRKNEELANKVLGPASDQIKLATGSEMEISKLPG
YVLETKVHGELASRVESELLCRAYFGIYLGEITMECYKESKEMFGQSMLSLF
CHIL4
(SEQ ID NO: 53)
MENNMVMVHEIPFPPEIKTTKPLSLLGYGITDMEIHFLQVKFTAIGVY
LDSDVVKHLQQWKGKKGNELAEDDDFFDALISAPVEKYLRIVVIKEIKGSQYGVQLE
SSVRDRLAAEDMYEEEEEAALEKIVEFLQSKYFKKDTLITFHFPATSPTAEIVVTLEGK
EESKLKVENKNVVDMIKKWYLGGTSGVSPSTISSLADNLSAELSK
CHIL5
(SEQ ID NO: 54)
MAVPEVVVEGVVFPPVARPPGSAGSHFLGGAGVRGIEIGGNFIKFTAI
GVYLEDAAVSALAKKWAGKTADELASDAAFFRDVVTGDFEKFTRVTMLLPLTGEQ
YAGKVTENCVAFWKAVGLYTDAEGVAVEKFKEAFKPETFPPGASILFTHSSTGVLTV
AFSKDSSVPASGGVAIENKHLCEAVLESIIGEHGVSPAAKLSLAARVSELLTKGTAGA
ADAPQAEPVSVTA
CHIL6
(SEQ ID NO: 55)
MLISAVGSETKTITFEGIPFPAEITAAGNPLSLLATGITDIEIHFLQIKYN
AIGVYLHSNDDSDLLTTHLGAWKGKTAEDLLADAAFWSALVSSPVEKLLRVVVIKEI
KGSQYGVQLESSVRDRLAAVDLYEDDEEEALEKVAEFFQAKYFKPGSVITFHFPATP
GPADITFVTEGKADAKITVENEHVAGMIQKWYLGGDNAVSPTTVRSLADRFAALLA
A
CHIL7
(SEQ ID NO: 56)
MGTEMATVEVEGIPFPQEITRTKPLSFLAHGVTDIEIHFLQIKYNAIGV
YLDKESVLGHLESWKGKKAEELVQDAGFFQALVFAPVEKLFRIVVIKEIKGSQYGVQ
LESSVRDRLVAVDKFEEEEEEALEKVTEFFQYKYFKPNSVLTFHFPTTPGIAEISFVTE
GKSEAKLTVDNNNVAEMIQKWYLGGESAVSPTTVKSLADQFAPLLSA
CHIL8
(SEQ ID NO: 57)
MATVEVEGIPFPQEITVSKPLSLLAHGITDIEIHFLQIKYNAIGVYLEKD
NVLGHLESWKGKKAEELVQDDGFFQALVSAPVEKLFRIVVIKEIKGSQYGVQLESSV
RDRLVSVDKYEDEEEESLEKVTEFFQSKYFKPNSVLTFHFPNTPGIAEISFVTEGKGEA
KLTVENKNVAEMIQKWYLGGESAVSPTTVKSLADQFAALLSA
F16
(SEQ ID NO: 60)
GGGGS
F17
(SEQ ID NO: 61)
GGGGSAEAAAKAEAAAKAAEAAAKAEAAAKAGGGGS
F18
(SEQ ID NO: 62)
GGGGSGGGGSAEAAAKAEAAAKAAEAAAKAEAAAKAGGGGSGGG
GS
HCS2-F16-PKS1
(SEQ ID NO: 63)
MASEENDLVFPSKEFSGQALVSSPQQYMEMHKRSMDDPAAFWSDIA
SEFYWKQKWGDQVFSENLDVRKGPISIEWFKGGITNICYNCLDKNVEAGLGDKTAIH
WEGNELGVDASLTYSELLQRVCQLANYLKDNGVKKGDAVVIYLPMLMELPIAMLA
CARIGAVHSVVFAGFSADSLAQRIVDCKPNVILTCNAVKRGPKTINLKAIVDAALDQS
SKDGVSVGICLTYDNSLATTRENTKWQNGRDVWWQDVISQYPTSCEVEWVDAEDP
LFLLYTSGSTGKPKGVLHTTGGYMIYTATTFKYAFDYKSTDVYWCTADCGWIGGHS
YVTYGPMLNGATVVVFEGAPNYPDPGRCWDIVDKYKVSIFYTAPTLVRSLMRDDDK
FVTRHSRKSLRVLGSAGEPINPSAWRWFFNVVGDSRCPISDTWGQTETGGFMITPLPG
AWPQKPGSATFPFFGVQPVIVDEKGNEIEGECSGYLCVKGSWPGAFRTLFGDHERYE
TTYFKPFAGYYFSGDGCSRDKDGYYWLTGRVDDVINVSGHRIGTAEVESALVLHPQ
CAEAAVVGIEHEVKGQGIYAFVTLLEGVPYSEELRKSLVLMVRNQIGAFAAPDRIHW
APGLPKTRSGKIMRRILRKIASRQLEELGDTSTLADPSVVDQLIALADGGGGSMNHLR
AEGPASVLAIGTANPENILLQDEFPDYYFRVTKSEHMTQLKEKFRKICDKSMIRKRNC
FLNEEHLKQNPRLVEHEMQTLDARQDMLVVEVPKLGKDACAKAIKEWGQPKSKITH
LIFTSASTTDMPGADYHCAKLLGLSPSVKRVMMYQLGCYGGGTVLRIAKDIAENNK
GARVLAVCCDIMACLFRGPSESDLELLVGQAIFGDGAAAVIVGAEPDESVGERPIFEL
VSTGQTILPNSEGTIGGHIREAGLIFDLHKDVPMLISNNIEKCLIEAFTPIGISDWNSIFWI
THPGGKAILDKVEEKLHLKSDKFVDSRHVLSEHGNMSSSTVLFVMDELRKRSLEEGK
STTGDGFEWGVLFGFGPGLTVERVVVRSVPIK
HCS2-F17-PKS1
(SEQ ID NO: 64)
MASEENDLVFPSKEFSGQALVSSPQQYMEMHKRSMDDPAAFWSDIA
SEFYWKQKWGDQVFSENLDVRKGPISIEWFKGGITNICYNCLDKNVEAGLGDKTAIH
WEGNELGVDASLTYSELLQRVCQLANYLKDNGVKKGDAVVIYLPMLMELPIAMLA
CARIGAVHSVVFAGFSADSLAQRIVDCKPNVILTCNAVKRGPKTINLKAIVDAALDQS
SKDGVSVGICLTYDNSLATTRENTKWQNGRDVWWQDVISQYPTSCEVEWVDAEDP
LFLLYTSGSTGKPKGVLHTTGGYMIYTATTFKYAFDYKSTDVYWCTADCGWIGGHS
YVTYGPMLNGATVVVFEGAPNYPDPGRCWDIVDKYKVSIFYTAPTLVRSLMRDDDK
FVTRHSRKSLRVLGSAGEPINPSAWRWFFNVVGDSRCPISDTWGQTETGGFMITPLPG
AWPQKPGSATFPFFGVQPVIVDEKGNEIEGECSGYLCVKGSWPGAFRTLFGDHERYE
TTYFKPFAGYYFSGDGCSRDKDGYYWLTGRVDDVINVSGHRIGTAEVESALVLHPQ
CAEAAVVGIEHEVKGQGIYAFVTLLEGVPYSEELRKSLVLMVRNQIGAFAAPDRIHW
APGLPKTRSGKIMRRILRKIASRQLEELGDTSTLADPSVVDQLIALADGGGGSAEAAA
KAEAAAKAAEAAAKAEAAAKAGGGGSMNHLRAEGPASVLAIGTANPENILLQDEFP
DYYFRVTKSEHMTQLKEKFRKICDKSMIRKRNCFLNEEHLKQNPRLVEHEMQTLDA
RQDMLVVEVPKLGKDACAKAIKEWGQPKSKITHLIFTSASTTDMPGADYHCAKLLG
LSPSVKRVMMYQLGCYGGGTVLRIAKDIAENNKGARVLAVCCDIMACLFRGPSESD
LELLVGQAIFGDGAAAVIVGAEPDESVGERPIFELVSTGQTILPNSEGTIGGHIREAGLI
FDLHKDVPMLISNNIEKCLIEAFTPIGISDWNSIFWITHPGGKAILDKVEEKLHLKSDKF
VDSRHVLSEHGNMSSSTVLFVMDELRKRSLEEGKSTTGDGFEWGVLFGFGPGLTVER
VVVRSVPIK
HCS2-F18-PKS1
(SEQ ID NO: 65)
MASEENDLVFPSKEFSGQALVSSPQQYMEMHKRSMDDPAAFWSDIA
SEFYWKQKWGDQVFSENLDVRKGPISIEWFKGGITNICYNCLDKNVEAGLGDKTAIH
WEGNELGVDASLTYSELLQRVCQLANYLKDNGVKKGDAVVIYLPMLMELPIAMLA
CARIGAVHSVVFAGFSADSLAQRIVDCKPNVILTCNAVKRGPKTINLKAIVDAALDQS
SKDGVSVGICLTYDNSLATTRENTKWQNGRDVWWQDVISQYPTSCEVEWVDAEDP
LFLLYTSGSTGKPKGVLHTTGGYMIYTATTFKYAFDYKSTDVYWCTADCGWIGGHS
YVTYGPMLNGATVVVFEGAPNYPDPGRCWDIVDKYKVSIFYTAPTLVRSLMRDDDK
FVTRHSRKSLRVLGSAGEPINPSAWRWFFNVVGDSRCPISDTWGQTETGGFMITPLPG
AWPQKPGSATFPFFGVQPVIVDEKGNEIEGECSGYLCVKGSWPGAFRTLFGDHERYE
TTYFKPFAGYYFSGDGCSRDKDGYYWLTGRVDDVINVSGHRIGTAEVESALVLHPQ
CAEAAVVGIEHEVKGQGIYAFVTLLEGVPYSEELRKSLVLMVRNQIGAFAAPDRIHW
APGLPKTRSGKIMRRILRKIASRQLEELGDTSTLADPSVVDQLIALADGGGGSGGGGS
AEAAAKAEAAAKAAEAAAKAEAAAKAGGGGSGGGGSMNHLRAEGPASVLAIGTA
NPENILLQDEFPDYYFRVTKSEHMTQLKEKFRKICDKSMIRKRNCFLNEEHLKQNPRL
VEHEMQTLDARQDMLVVEVPKLGKDACAKAIKEWGQPKSKITHLIFTSASTTDMPG
ADYHCAKLLGLSPSVKRVMMYQLGCYGGGTVLRIAKDIAENNKGARVLAVCCDIM
ACLFRGPSESDLELLVGQAIFGDGAAAVIVGAEPDESVGERPIFELVSTGQTILPNSEG
TIGGHIREAGLIFDLHKDVPMLISNNIEKCLIEAFTPIGISDWNSIFWITHPGGKAILDKV
EEKLHLKSDKFVDSRHVLSEHGNMSSSTVLFVMDELRKRSLEEGKSTTGDGFEWGV
LFGFGPGLTVERVVVRSVPIK
P3
(SEQ ID NO: 66)
MGSSPEDEIQQLEEEIAQLEQKNAALKEKNQALKYGS
P4
(SEQ ID NO: 67)
GSPEDKIAQLKQKIQALKQENQQLEEENAALEYGGSG
PKS1.1
(SEQ ID NO: 68)
MNHLRAEGPASVLAIGTANPENILLQDEFPDYYFRVTKSEHMTQLKE
KFRKICDKSMIRKRNCFLNEEHLKQNPRLVEHEMQTLDARQDMLVVEVPKLGKDAC
AKAIKEWGQPKSKITHLIFTSASTTDMPGADYHCAKLLGLSPSVKRVMMYQLGCYG
GGTVLRIAKDIAENNKGARVLAVCCDIMACLFRGPSESDLELLVGQAIFGDGAAAVI
VGAEPDESVGERPIFELVSTGQTILPNSEGTIGGHIREAGLIFDLHKDVPMLISNNIEKC
LIEAFTPIGISDWNSIFWITHPGGKAILDKVEEKLHLKSDKFVDSRHVLSEHGNMSSSC
VLFVMDELRKRSLEEGKSTTGDGFEWGVLFGFGPGLTVERVVVRSVPIKY
PKC4.11
(SEQ ID NO: 69)
MAVKHLIVLKFKDEITEAQKEEFFKTYVNLVNIIPAMKDVYWGKLEV
NNKLGNGGYTHIVEVTFESVETIQDYIIHPAHVGFGDVYRSFWEKLLIFDYTPRK
PKC4.15
(SEQ ID NO: 70)
MGEANKGVVKHLIVLKFKDEITEAQKEEFFKTYVNLVNIIPAMKDVY
WGKDVTQKNKEEGYTHIVEVTFESVETIQDYIIHPAHVGFGDVYRSFWEKLLIFDYTP
TIVLPNSSY
PKC4.17
(SEQ ID NO: 71)
MGEANKGVVKHLIVLKFKDEITEAQKEEFFKTYVNLVNIIPAMKDVY
WGKLEVNNKLGNGGYTHIVEVTFESVETIQDYIIHPAHVGFGDVYRSFWEKLLIFDYT
PTIVLPNSSY
PKC4.19
(SEQ ID NO: 72)
MAVKHVIILKFKEGITEAQKEEFFKTYVNLVNLVPAMKAVQWGKLE
VNNKLGNGGYTHIVESTFESVETIQDYIIHPAHVGFGDVYRSFWEKLLIFDYTPRK
PKC4.30
(SEQ ID NO: 73)
MAVKHVIILKFKEGITEAQKEEFFKTYVNLVNLVPAMKAVQWGKLE
VNNKLGNGGYTHIVESTFESVETIQDYIIHPAHVGFGDVYRSFWEKLLIFDYTPTIVLP
NSSY
PKC4.31
(SEQ ID NO: 74)
MGEANKGVVKHVIILKFKEGITEAQKEEFFKTYVNLVNLVPAMKAV
QWGKLEVNNKLGNGGYTHIVESTFESVETIQDYIIHPAHVGFGDVYRSFWEKLLIFDY
TPRK
PKC4.32
(SEQ ID NO: 75)
MSPEDENRELEEKIRELKEKNEELKREIKYLEEGVVKHVIILKFKEGIT
EAQKEEFFKTYVNLVNLVPAMKAVQWGKLEVNNKLGNGGYTHIVESTFESVETIQD
YIIHPAHVGFGDVYRSFWEKLLIFDYTPTIVLPNSSYSPEDKNEELKREIERLEEENREL
ERKIEYLKR
PKC4.33
(SEQ ID NO: 76)
MGEANKGVVKHVIILKFKEGITEAQKEEMFKTYVNLVNLVPAMKAV
QWGKLEVNNKLGNGGYTHIVESTFESVETIQDYIIHPAHVGFGDVYRSFWEKLLIFDY
TPTIVLPNSSY
PKC4.35
(SEQ ID NO: 77)
MAVKHVIILKFKEGITEAQKEEMFKTYVNLVNLVPAMKAVQWGKLE
VNNKLGNGGYTHIVESTFESVETIQDYIIHPAHVGFGDVYRSFWEKLLIFDYTPTIVLP
NSSY
PKC4.36
(SEQ ID NO: 78)
MGEANKGVVKHVIILKFKEGITEAQKEEMFKTYVNLVNLVPAMKAV
QWGKLEVNNKLGNGGYTHIVESTFESVETIQDYIIHPAHVGFGDVYRSFWEKLLIFDY
TPRK
PKC4.37
(SEQ ID NO: 79)
MAVKHVIILKFKEGITEAQKEEMFKTYVNLVNLVPAMKAVQWGKLE
VNNKLGNGGYTHIVESTFESVETIQDYIIHPAHVGFGDVYRSFWEKLLIFDYTPRK
PKC4.38
(SEQ ID NO: 80)
MSPEDENRELEEKIRELKEKNEELKREIKYLEEGVVKHVIILKFKEGIT
EAQKEEMFKTYVNLVNLVPAMKAVQWGKLEVNNKLGNGGYTHIVESTFESVETIQD
YIIHPAHVGFGDVYRSFWEKLLIFDYTPTIVLPNSSYSPEDKNEELKREIERLEEENREL
ERKIEYLKR

1-84. (canceled)

85. A polyketide synthase comprising an amino acid sequence with at least 70% identity to SEQ ID NO: 1, 2, 3, 4, 5, 6, 7, 8, or 68, wherein the polyketide synthase has polyketide synthase (PKS) activity, wherein the polyketide synthase is capable of producing a tetraketide from one or more acyl-CoA from carboxylic acids with two to twenty-two carbons, and wherein the polyketide synthase is capable of producing the tetraketide from the acyl-CoA substrate at a higher rate than PKS1 from Cannabis sativa.

86. The polyketide synthase of claim 85, comprising an amino acid sequence with at least one amino acid substitution as compared to SEQ ID NO: 2, wherein the amino acid substitution is located in SEQ ID NO: 2 at positions selected from A106, Y140, S141, A145, L169, G171, C172, E200, T202, I204, A205, G208, G219, F223, G224, D225, G226, I263, M265, M272, Y274, H313, G315, N346, S348, F382, G383, and P384.

87. The polyketide synthase of claim 85, comprising an amino acid sequence with at least one amino acid substitution as compared to SEQ ID NO: 3, wherein the amino acid substitution is located in SEQ ID NO: 3 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

88. The polyketide synthase of claim 85, comprising an amino acid sequence with at least one amino acid substitution as compared to SEQ ID NO: 4, wherein the amino acid substitution is located in SEQ ID NO: 4 at positions selected from A102, Y136, S137, A141, L165, G167, C168, E196, T198, I200, A201, G204, G215, F219, G220, D221, G222, I258, M260, M267, Y269, H308, G310, N341, S343, F377, G378, and P379.

89. The polyketide synthase of claim 85, comprising an amino acid sequence with at least one amino acid substitution as compared to SEQ ID NO: 5, wherein the amino acid substitution is located in SEQ ID NO: 5 at positions selected from A108, Y142, S143, A147, L171, G173, C174, E202, T204, I206, A207, G210, G221, F225, G226, D227, G228, I264, M266, M273, Y275, H314, G316, N347, S349, F383, G384, and P385.

90. The polyketide synthase of claim 85, comprising an amino acid sequence with at least one amino acid substitution as compared to SEQ ID NO: 6, wherein the amino acid substitution is located in SEQ ID NO: 6 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

91. The polyketide synthase of claim 85, comprising an amino acid sequence with at least one amino acid substitution as compared to SEQ ID NO: 7, wherein the amino acid substitution is located in SEQ ID NO: 7 at positions selected from A103, Y136, S137, A141, L165, G167, C168, E196, T198, I200, A201, G204, G215, F219, G220, D221, G222, I258, M260, M267, Y269, H309, G311, N342, S344, F379, G380, and P381.

92. The polyketide synthase of claim 85, comprising an amino acid sequence with at least one amino acid substitution as compared to SEQ ID NO: 8, wherein the amino acid substitution is located in SEQ ID NO: 8 at positions selected from A103, Y137, S138, A142, L166, G168, C169, E197, T199, I201, A202, G205, G216, F220, G221, D222, G223, I259, M261, M268, Y270, H309, G311, N342, S344, F376, G377, and P378.

93. The polyketide synthase of claim 85, wherein the polyketide cyclase is a fusion protein that comprises a polypeptide having polyketide cyclase activity.

94. The polyketide synthase of claim 93, wherein the fusion protein is capable of producing a ratio of olivetolic acid to olivetol from Hexanoyl-CoA at a ratio of greater than 0.1.

95. A polyketide cyclase comprising an amino acid sequence with at least 90% identity to SEQ ID NO: 9, 10, 11, 12, 69, 70, 71, 72, 73, 74, 75, 76, 77, 78, 79, or 80, wherein the polyketide cyclase has polyketide cyclase (PKC) activity, wherein the polyketide cyclase is capable of producing olivetolic acid (OA), an OA analog, divarinic acid (DVA), or a DVA analog from a tetraketide at a higher rate than PKC4 (SEQ ID NO: 10).

96. The polyketide cyclase of claim 95, wherein the amino acid sequence of the polyketide cyclase comprises at least one amino acid modification as compared to SEQ ID NO: 9, 10, 11, 12, 69, 70, 71, 72, 73, 74, 75, 76, 77, 78, 79 or 80.

97. The polyketide cyclase of claim 96, comprising a chimeric amino acid sequence comprising portions of SEQ ID NO: 9 and SEQ ID NO: 10.

98. The polyketide cyclase of claim 96, comprising an amino acid sequence with at least one amino acid modification as compared to SEQ ID NO: 10, wherein the amino acid modification is a substitution located in SEQ ID NO: 10 at positions selected from V9, H11 V12, F13, I14, L15, M17, M29, N30, Y33, A45, Q47, L51, E52, N54-Y62, H64, I65, F66, E67, S68, I69, F70, M73, I76, Y79, I80, L86, L88, R89, Y92, F93, L96, F99, L100, V101, and F102, D103 and K105, or wherein the modification is a 1-20 amino acid C-terminus or N-terminus truncation of SEQ ID NO: 10.

99. The polyketide cyclase of claim 96, comprising an amino acid sequence with at least one amino acid modification as compared to SEQ ID NO: 11, 69 or 76, or wherein the at least one amino acid modification is a substitution located in SEQ ID NO: 11 at positions selected from V9, H11, V12, I13 I14, L15, F17, F29, F30, Y33, A45, Q47, L51, E52, N54-Y62, H64, I65, V66, E67, S68, I69, F70, V73, I76, Y79, I80, V86, F88, G89, Y92, R93, W96, L99, L100, I101, F102, D103, and T105, or wherein the at least one amino acid modification is a substitution located in SEQ ID NO: 69 at positions selected from V3, H5, L9, Y27, L45, E46, N48-Y56, H58, I59, E61, T63, F64, I70, Y73, I74, Y86, L94, F96, and D97, wherein the at least one amino acid modification is a substitution is located in SEQ ID NO: 76 at positions selected from V9, H11 V12, I14, L15, M29, Y33, A45, Q47, L51, E52, N54-Y62, H64, I65, E67, S68, F70, I76, Y79, I80, Y92, L100, F102, and D103, or wherein the amino acid modification is a 1-20 C-terminus or N-terminus truncation as compared to SEQ ID NO: 11, 69 or 76.

100. A cell comprising an exogenous nucleotide sequence coding for at least one of the following:
a. a polyketide synthase comprising an amino acid sequence with at least 90% identity to SEQ ID NO: 1, 2, 3, 4, 5, 6, 7, 8, or 68, wherein the polyketide synthase is capable of producing a tetraketide from one or more acyl-CoA from carboxylic acids with two to twenty-two carbons, and wherein the polyketide synthase is capable of producing the tetraketide from the acyl-CoA substrate at a higher rate than PKS1 from Cannabis sativa;
b. a polyketide cyclase comprising an amino acid sequence with at least 90% identity to SEQ ID NO: 9, 10, 11, 12, 69, 70, 71, 72, 73, 74, 75, 76, 77, 78, 79, or 80, wherein the polyketide cyclase has polyketide cyclase (PKC) activity, wherein the polyketide cyclase is capable of producing olivetolic acid (OA), an OA analog, divarinic acid (DVA), or a DVA analog from a tetraketide at a higher rate than PKC4 (SEQ ID NO: 10); and
c. a fusion protein comprising a polypeptide having polyketide synthase activity and either a polypeptide having polyketide cyclase activity or a polypeptide having acyl synthetase activity.

101. The cell of claim 100, comprising the exogenous nucleotides encoding the fusion protein (c), wherein the fusion protein is capable of producing olivetolic acid from Hexanoyl-CoA or divarinic acid from Butyryl-CoA

102. The cell of claim 100, comprising the exogenous nucleotides encoding the fusion protein (c) comprising a polypeptide having acyl-CoA synthetase activity, wherein the polypeptide comprises an amino acid sequence selected from the group consisting of SEQ ID NO: 28, 29, 30, 31, 32, and 33.

103. The cell of claim 100, comprising the exogenous nucleotides encoding the fusion protein (c), wherein the fusion protein comprises an amino acid sequence selected from the group consisting of SEQ ID NO: 34, 35, 36, 37, or 38.

104. The cell of claim 100, wherein the cell is a bacteria cell or a yeast cell.
